SEQUENCE LISTING (248 sequences)

SEQ ID NO: 1 - protein, 735 aa, Artificial sequence
  Synthetic polypeptide
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFHHHHHHSFITQYSTGQVSVEIEWELQKENSKRWNPEIQYTSNYNKSVNVDFTVDTNGVYSEPRPIGTRYLTRNL

SEQ ID NO: 2 - protein, 736 aa, Artificial sequence
  Synthetic polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFHHHHHHSFITQYSTGQVSVEIEWELQKENSKRWNPEIQYTSNYYKSNNVEFAVNTEGVYSEPRPIGTRYLTRNL

SEQ ID NO: 3 - protein, 31 aa, adeno-associated virus 2
IKNTPVPANPSTTFSAAKFASFITQYSTGQV

SEQ ID NO: 4 - protein, 31 aa, adeno-associated virus 1
IKNTPVPANPPAEFSATKFASFITQYSTGQV

SEQ ID NO: 5 - protein, 31 aa, adeno-associated virus 4
IKNTPVPANPATTFSSTPVNSFITQYSTGQV

SEQ ID NO: 6 - protein, 30 aa, adeno-associated virus 5
IKNTPVPGNITSFSDVPVSSFITQYSTGQV

SEQ ID NO: 7 - protein, 31 aa, adeno-associated virus 8
IKNTPVPADPPTTFNQSKLNSFITQYSTGQV

SEQ ID NO: 8 - protein, 21 aa, Artificial sequence
  Synthetic peptide
IKNTPVPANPSFITQYSTGQV

SEQ ID NO: 9 - protein, 31 aa, Artificial sequence
  Synthetic peptide
IKNTPVPANPGGGGGGGGGGSFITQYSTGQV

SEQ ID NO: 10 - protein, 13 aa, adeno-associated virus 2
ANPSTTFSAAKFA

SEQ ID NO: 11 - protein, 12 aa, adeno-associated virus 5
GNITSFSDVPVS

SEQ ID NO: 12 - protein, 10 aa, adeno-associated virus 2
STTFSAAKFA

SEQ ID NO: 13 - protein, 10 aa, Artificial sequence
  Synthetic peptide
VNTANSTKFA

SEQ ID NO: 14 - protein, 10 aa, Artificial sequence
  Synthetic peptide
QPEHSSTKFA

SEQ ID NO: 15 - protein, 10 aa, Artificial sequence
  Synthetic peptide
SIGYPLPKFA

SEQ ID NO: 16 - protein, 10 aa, Artificial sequence
  Synthetic peptide
RGDFSAAKFA

SEQ ID NO: 17 - protein, 10 aa, Artificial sequence
  Synthethic peptide
STRGDAAKFA

SEQ ID NO: 18 - protein, 10 aa, Artificial sequence
  Synthetic peptide
STTFRGDKFA

SEQ ID NO: 19 - protein, 10 aa, Artificial sequence
  Synthetic peptide
SGRGDSAKFA

SEQ ID NO: 20 - protein, 10 aa, adeno-associated virus 1
PAEFSATKFA

SEQ ID NO: 21 - protein, 10 aa, adeno-associated virus 4
AATFSSTPVN

SEQ ID NO: 22 - protein, 9 aa, adeno-associated virus 5
TSFSDVPVS

SEQ ID NO: 23 - protein, 10 aa, adeno-associated virus 8
STTFNSQKLN

SEQ ID NO: 24 - protein, 10 aa, Artificial sequence
  Synthetic peptide
STTFHHHHHH

SEQ ID NO: 25 - protein, 4 aa, Artificial sequence
  Synthetic peptide
EPDW

SEQ ID NO: 26 - protein, 8 aa, Artificial sequence
  Synthetic peptide
AWRHPQGG

SEQ ID NO: 27 - protein, 6 aa, Artificial sequence
  Synthetic peptide
GDWVFI

SEQ ID NO: 28 - protein, 8 aa, Artificial sequence
  Synthetic peptide
WXHPQFXX

SEQ ID NO: 29 - protein, 8 aa, Artificial sequence
  Synthetic peptide
WSHPQFEK

SEQ ID NO: 30 - protein, 5 aa, Artificial sequence
  Synthetic peptide
FVFLP

SEQ ID NO: 31 - protein, 4 aa, Artificial sequence
  Synthetic peptide
XXXX

SEQ ID NO: 32 - protein, 4 aa, Artificial sequence
  Synthetic peptide
RGNR

SEQ ID NO: 33 - protein, 7 aa, Artificial sequence
  Synthetic peptide
NSVRDLX

SEQ ID NO: 34 - protein, 7 aa, Artificial sequence
  Synthetic peptide
PRSVTVP

SEQ ID NO: 35 - protein, 7 aa, Artificial sequence
  Synthetic peptide
NSVSSXX

SEQ ID NO: 36 - protein, 6 aa, Artificial sequence
  Synthetic peptide
NGRAHA

SEQ ID NO: 37 - protein, 7 aa, Artificial sequence
  Synthetic peptide
QPEHSST

SEQ ID NO: 38 - protein, 7 aa, Artificial sequence
  Synthetic peptide
VNTANST

SEQ ID NO: 39 - protein, 7 aa, Artificial sequence
  Synthetic peptide
HGPMQKS

SEQ ID NO: 40 - protein, 7 aa, Artificial sequence
  Synthetic peptide
PHKPPLA

SEQ ID NO: 41 - protein, 7 aa, Artificial sequence
  Synthetic peptide
IKNNEMW

SEQ ID NO: 42 - protein, 7 aa, Artificial sequence
  Synthetic peptide
RNLDTPM

SEQ ID NO: 43 - protein, 7 aa, Artificial sequence
  Synthetic peptide
VDSHRQS

SEQ ID NO: 44 - protein, 7 aa, Artificial sequence
  Synthetic peptide
YDSKTKT

SEQ ID NO: 45 - protein, 7 aa, Artificial sequence
  Synthetic peptide
SQLPHQK

SEQ ID NO: 46 - protein, 7 aa, Artificial sequence
  Synthetic peptide
STMQQNT

SEQ ID NO: 47 - protein, 7 aa, Artificial sequence
  Synthetic peptide
TERYMTQ

SEQ ID NO: 48 - protein, 7 aa, Artificial sequence
  Synthetic peptide
DASLSTS

SEQ ID NO: 49 - protein, 7 aa, Artificial sequence
  Synthetic peptide
DLPNKKT

SEQ ID NO: 50 - protein, 7 aa, Artificial sequence
  Synthetic peptide
DLTAARL

SEQ ID NO: 51 - protein, 7 aa, Artificial sequence
  Synthetic peptide
EPHQFNY

SEQ ID NO: 52 - protein, 7 aa, Artificial sequence
  Synthetic peptide
EPQSNHT

SEQ ID NO: 53 - protein, 7 aa, Artificial sequence
  Synthetic peptide
MSSWPSQ

SEQ ID NO: 54 - protein, 7 aa, Artificial sequence
  Synthetic peptide
NPKHNAT

SEQ ID NO: 55 - protein, 7 aa, Artificial sequence
  Synthetic peptide
PDGMRTT

SEQ ID NO: 56 - protein, 7 aa, Artificial sequence
  Synthetic peptide
PNNNKTT

SEQ ID NO: 57 - protein, 7 aa, Artificial sequence
  Synthetic peptide
QSTTHDS

SEQ ID NO: 58 - protein, 7 aa, Artificial sequence
  Synthetic peptide
TGSKQKQ

SEQ ID NO: 59 - protein, 7 aa, Artificial sequence
  Synthetic peptide
SLKHQAL

SEQ ID NO: 60 - protein, 7 aa, Artificial sequence
  Synthetic peptide
SPIDGEQ

SEQ ID NO: 61 - protein, 8 aa, Artificial sequence
  Synthetic peptide
WIFPWIQL

SEQ ID NO: 62 - protein, 9 aa, Artificial sequence
  Synthetic peptide
CDCRGDCFC

SEQ ID NO: 63 - protein, 5 aa, Artificial sequence
  Synthetic peptide
CNGRC

SEQ ID NO: 64 - protein, 7 aa, Artificial sequence
  Synthetic peptide
CPRECES

SEQ ID NO: 65 - protein, 10 aa, Artificial sequence
  Synthetic peptide
CTTHWGFTLC

SEQ ID NO: 66 - protein, 9 aa, Artificial sequence
  Synthetic peptide
CGRRAGGSC

SEQ ID NO: 67 - protein, 9 aa, Artificial sequence
  Synthetic peptide
CKGGRAKDC

SEQ ID NO: 68 - protein, 9 aa, Artificial sequence
  Synthetic peptide
CVPELGHEC

SEQ ID NO: 69 - protein, 9 aa, Artificial Sequence
  Synthetic peptide
CRRETAWAK

SEQ ID NO: 70 - protein, 14 aa, Artificial Sequence
  Synthetic peptide
VSWFSHRYSPFAVS

SEQ ID NO: 71 - protein, 13 aa, Artificial Sequence
  Synthetic peptide
GYRDGYAGPILYN

SEQ ID NO: 72 - protein, 7 aa, Artificial Sequence
  Synthetic peptide
XXXYXXX

SEQ ID NO: 73 - protein, 4 aa, Artificial Sequence
  Synthetic peptide
YXNW

SEQ ID NO: 74 - protein, 7 aa, Artificial Sequence
  Synthetic peptide
RPLPPLP

SEQ ID NO: 75 - protein, 7 aa, Artificial Sequence
  Synthetic peptide
APPLPPR

SEQ ID NO: 76 - protein, 12 aa, Artificial Sequence
  Synthetic peptide
DVFYPYPYASGS

SEQ ID NO: 77 - protein, 6 aa, Artificial Sequence
  Synthetic peptide
MYWYPY

SEQ ID NO: 78 - protein, 12 aa, Artificial Sequence
  Synthetic peptide
DITWDQLWDLMK

SEQ ID NO: 79 - protein, 8 aa, Artificial Sequence
  Synthetic peptide
CWDDXWLC

SEQ ID NO: 80 - protein, 14 aa, Artificial Sequence
  Synthetic peptide
EWCEYLGGYLRCYA

SEQ ID NO: 81 - protein, 14 aa, Artificial Sequence
  Synthetic peptide
YXCXXGPXTWXCXP

SEQ ID NO: 82 - protein, 14 aa, Artificial Sequence
  Synthetic peptide
IEGPTLRQWLAARA

SEQ ID NO: 83 - protein, 5 aa, Artificial Sequence
  Synthetic peptide
LWXXX

SEQ ID NO: 84 - protein, 7 aa, Artificial Sequence
  Synthetic peptide
XFXXYLW

SEQ ID NO: 85 - protein, 13 aa, Artificial Sequence
  Synthetic peptide
SSIISHFRWGLCD

SEQ ID NO: 86 - protein, 13 aa, Artificial Sequence
  Synthetic peptide
MSRPACPPNDKYE

SEQ ID NO: 87 - protein, 8 aa, Artificial Sequence
  Synthetic peptide
CLRSGRGC

SEQ ID NO: 88 - protein, 9 aa, Artificial Sequence
  Synthetic peptide
CHWMFSPWC

SEQ ID NO: 89 - protein, 4 aa, Artificial Sequence
  Synthetic peptide
WXXF

SEQ ID NO: 90 - protein, 8 aa, Artificial Sequence
  Synthetic peptide
CSSRLDAC

SEQ ID NO: 91 - protein, 7 aa, Artificial Sequence
  Synthetic peptide
CLPVASC

SEQ ID NO: 92 - protein, 13 aa, Artificial Sequence
  Synthetic peptide
CGFECVRQCPERC

SEQ ID NO: 93 - protein, 13 aa, Artificial Sequence
  Synthetic peptide
CVALCREACGEGC

SEQ ID NO: 94 - protein, 9 aa, Artificial Sequence
  Synthetic peptide
SWCEPGWCR

SEQ ID NO: 95 - protein, 7 aa, Artificial Sequence
  Synthetic peptide
YSGKWGW

SEQ ID NO: 96 - protein, 7 aa, Artificial Sequence
  Synthetic peptide
GLSGGRS

SEQ ID NO: 97 - protein, 7 aa, Artificial Sequence
  Synthetic peptide
LMLPRAD

SEQ ID NO: 98 - protein, 9 aa, Artificial Sequence
  Synthetic peptide
CSCFRDVCC

SEQ ID NO: 99 - protein, 9 aa, Artificial Sequence
  Synthetic peptide
CRDVVSVIC

SEQ ID NO: 100 - protein, 6 aa, Artificial Sequence
  Synthetic peptide
MARSGL

SEQ ID NO: 101 - protein, 6 aa, Artificial Sequence
  Synthetic peptide
MARAKE

SEQ ID NO: 102 - protein, 6 aa, Artificial Sequence
  Synthetic peptide
MSRTMS

SEQ ID NO: 103 - protein, 6 aa, Artificial Sequence
  Synthetic peptide
KCCYSL

SEQ ID NO: 104 - protein, 14 aa, Artificial Sequence
  Synthetic peptide
MYWGDSHWLQYWYE

SEQ ID NO: 105 - protein, 7 aa, Artificial Sequence
  Synthetic peptide
MQLPLAT

SEQ ID NO: 106 - protein, 4 aa, Artificial Sequence
  Synthetic peptide
EWLS

SEQ ID NO: 107 - protein, 4 aa, Artificial Sequence
  Synthetic peptide
SNEW

SEQ ID NO: 108 - protein, 4 aa, Artificial Sequence
  Synthetic peptide
TNYL

SEQ ID NO: 109 - protein, 12 aa, Artificial Sequence
  Synthetic peptide
WDLAWMFRLPVG

SEQ ID NO: 110 - protein, 13 aa, Artificial Sequence
  Synthetic peptide
CTVALPGGYVRVC

SEQ ID NO: 111 - protein, 9 aa, Artificial Sequence
  Synthetic peptide
CVPELGHEC

SEQ ID NO: 112 - protein, 9 aa, Artificial Sequence
  Synthetic peptide
CGRRAGGSC

SEQ ID NO: 113 - protein, 13 aa, Artificial Sequence
  Synthetic peptide
CVAYCIEHHCWTC

SEQ ID NO: 114 - protein, 12 aa, Artificial Sequence
  Synthetic peptide
CVFAHNYDYLVC

SEQ ID NO: 115 - protein, 10 aa, Artificial Sequence
  Synthetic peptide
CVFTSNYAFC

SEQ ID NO: 116 - protein, 7 aa, Artificial Sequence
  Synthetic peptide
VHSPNKK

SEQ ID NO: 117 - protein, 7 aa, Artificial Sequence
  Synthetic peptide
CRGDGWC

SEQ ID NO: 118 - protein, 6 aa, Artificial Sequence
  Synthetic peptide
XRGCDX

SEQ ID NO: 119 - protein, 5 aa, Artificial Sequence
  Synthetic peptide
PXXST

SEQ ID NO: 120 - protein, 10 aa, Artificial Sequence
  Synthetic peptide
CTTHWGFTLC

SEQ ID NO: 121 - protein, 11 aa, Artificial Sequence
  Synthetic peptide
SGKGPRQITAL

SEQ ID NO: 122 - protein, 14 aa, Artificial Sequence
  Synthetic peptide
AAAAAAAAAXXXXX

SEQ ID NO: 123 - protein, 6 aa, Artificial Sequence
  Synthetic peptide
VYMSPF

SEQ ID NO: 124 - protein, 7 aa, Artificial Sequence
  Synthetic peptide
ATWLPPR

SEQ ID NO: 125 - protein, 12 aa, Artificial Sequence
  Synthetic peptide
HTMYYHHYQHHL

SEQ ID NO: 126 - protein, 19 aa, Artificial Sequence
  Synthetic peptide
SEVGCRAGPLQWLCEKYFG

SEQ ID NO: 127 - protein, 18 aa, Artificial Sequence
  Synthetic peptide
CGLLPVGRPDRNVWRWLC

SEQ ID NO: 128 - protein, 15 aa, Artificial Sequence
  Synthetic peptide
CKGQCDRFKGLPWEC

SEQ ID NO: 129 - protein, 5 aa, Artificial Sequence
  Synthetic peptide
SGRSA

SEQ ID NO: 130 - protein, 4 aa, Artificial Sequence
  Synthetic peptide
WGFP

SEQ ID NO: 131 - protein, 17 aa, Artificial Sequence
  Synthetic peptide
AEPMPHSLNFSQYLWYT

SEQ ID NO: 132 - protein, 6 aa, Artificial Sequence
  Synthetic peptide
WAYXSP

SEQ ID NO: 133 - protein, 7 aa, Artificial Sequence
  Synthetic peptide
IELLQAR

SEQ ID NO: 134 - protein, 12 aa, Artificial Sequence
  Synthetic peptide
DITWDQLWDLMK

SEQ ID NO: 135 - protein, 16 aa, Artificial Sequence
  Synthetic peptide
AYTKCSRQWRTCMTTH

SEQ ID NO: 136 - protein, 15 aa, Artificial Sequence
  Synthetic peptide
PQNSKIPGPTFLDPH

SEQ ID NO: 137 - protein, 15 aa, Artificial Sequence
  Synthetic peptide
SMEPALPDWWWKMFK

SEQ ID NO: 138 - protein, 16 aa, Artificial Sequence
  Synthetic peptide
ANTPCGPYTHDCPVKR

SEQ ID NO: 139 - protein, 12 aa, Artificial Sequence
  Synthetic peptide
TACHQHVRMVRP

SEQ ID NO: 140 - protein, 12 aa, Artificial Sequence
  Synthetic peptide
VPWMEPAYQRFL

SEQ ID NO: 141 - protein, 8 aa, Artificial Sequence
  Synthetic peptide
DPRATPGS

SEQ ID NO: 142 - protein, 12 aa, Artificial Sequence
  Synthetic peptide
FRPNRAQDYNTN

SEQ ID NO: 143 - protein, 9 aa, Artificial Sequence
  Synthetic peptide
CTKNSYLMC

SEQ ID NO: 144 - protein, 11 aa, Artificial Sequence
  Synthetic peptide
CXXTXXXGXGC

SEQ ID NO: 145 - protein, 9 aa, Artificial Sequence
  Synthetic peptide
CPIEDRPMC

SEQ ID NO: 146 - protein, 12 aa, Artificial Sequence
  Synthetic peptide
HEWSYLAPYPWF

SEQ ID NO: 147 - protein, 9 aa, Artificial Sequence
  Synthetic peptide
MCPKHPLGC

SEQ ID NO: 148 - protein, 15 aa, Artificial Sequence
  Synthetic peptide
RMWPSSTVNLSAGRR

SEQ ID NO: 149 - protein, 20 aa, Artificial Sequence
  Synthetic peptide
SAKTAVSQRVWLPSHRGGEP

SEQ ID NO: 150 - protein, 20 aa, Artificial Sequence
  Synthetic peptide
KSREHVNNSACPSKRITAAL

SEQ ID NO: 151 - protein, 4 aa, Artificial Sequence
  Synthetic peptide
EGFR

SEQ ID NO: 152 - protein, 6 aa, Artificial Sequence
  Synthetic peptide
AGLGVR

SEQ ID NO: 153 - protein, 15 aa, Artificial Sequence
  Synthetic peptide
GTRQGHTMRLGVSDG

SEQ ID NO: 154 - protein, 15 aa, Artificial Sequence
  Synthetic peptide
IAGLATPGWSHWLAL

SEQ ID NO: 155 - protein, 7 aa, Artificial Sequence
  Synthetic peptide
SMSIARL

SEQ ID NO: 156 - protein, 7 aa, Artificial Sequence
  Synthetic peptide
HTFEPGV

SEQ ID NO: 157 - protein, 15 aa, Artificial Sequence
  Synthetic peptide
NTSLKRISNKRIRRK

SEQ ID NO: 158 - protein, 15 aa, Artificial sequence
  Synthetic peptide
LRIKRKRRKRKKTRK

SEQ ID NO: 159 - protein, 7 aa, Artificial sequence
  Synthetic peptide
SIGYPLP

SEQ ID NO: 160 - protein, 6 aa, Artificial sequence
  Synthetic peptide
SGRGDS

SEQ ID NO: 161 - protein, 6 aa, Artificial Sequence
  Synthetic peptide
SAAKFA

SEQ ID NO: 162 - DNA, 48 bp, Artificial Sequence
  Synthetic oligonucleotide
caccatcaccatcaccattccttcatcacacagtactccacgggacag

SEQ ID NO: 163 - DNA, 45 bp, Artificial Sequence
  Synthetic oligonucleotide
atggtgatggtgatggtggaaggtggtcgaaggattcgcaggtac

SEQ ID NO: 164 - DNA, 43 bp, Artificial Sequence
  Synthetic oligonucleotide
gaatccttcgaccaccttcagtcacgcaaagtttgcttccttc

SEQ ID NO: 165 - DNA, 45 bp, Artificial Sequence
  Synthetic oligonucleotide
cctgcgaatccttcgaccaccttcagtcaccacaagtttgcttcc

SEQ ID NO: 166 - DNA, 48 bp, Artificial Sequence
  Synthetic oligonucleotide
ccttcgaccaccttccaccaccacaagtttgcttccttcatcacacag

SEQ ID NO: 167 - DNA, 48 bp, Artificial Sequence
  Synthetic oligonucleotide
cctgcgaatccttcgaccaccttccatcaccaccactttgcttccttc

SEQ ID NO: 168 - DNA, 50 bp, Artificial Sequence
  Synthetic oligonucleotide
gcgaatccttcgaccaccttccatcaccaccaccacgcttccttcatcac

SEQ ID NO: 169 - DNA, 24 bp, Artificial Sequence
  Synthetic oligonucleotide
aavagcactctgattgacaaatac

SEQ ID NO: 170 - DNA, 22 bp, Artificial Sequence
  Synthetic oligonucleotide
ccttcgcttcaaaaaatggaac

SEQ ID NO: 171 - DNA, 31 bp, Artificial Sequence
  Synthetic oligonucleotide
tccttcatcacacagtactccacgggacagg

SEQ ID NO: 172 - DNA, 33 bp, Artificial Sequence
  Synthetic oligonucleotide
aggattcgcaggtaccggggtgttcttgatgag

SEQ ID NO: 173 - DNA, 61 bp, Artificial Sequence
  Synthetic oligonucleotide
ggaggaggaggaggaggaggaggaggaggatccttcatcacacagtactccacgggacagg

SEQ ID NO: 174 - DNA, 63 bp, Artificial Sequence
  Synthetic oligonucleotide
tcctcctcctcctcctcctcctcctcctccaggattcgcaggtaccggggtgttcttgatgag

SEQ ID NO: 175 - DNA, 36 bp, Artificial Sequence
  Synthetic oligonucleotide
gcgaatcctggaggaggattcggaggaggaggagga

SEQ ID NO: 176 - DNA, 33 bp, Artificial Sequence
  Synthetic oligonucleotide
ggaggaggaggaggaaagggaggatccttcatc

SEQ ID NO: 177 - DNA, 36 bp, Artificial Sequence
  Synthetic oligonucleotide
ggaggaggaggaggatttggatccttcatcacacag

SEQ ID NO: 178 - DNA, 30 bp, Artificial Sequence
  Synthetic oligonucleotide
agagatgtgtacctgcaggggcccatctgg

SEQ ID NO: 179 - DNA, 30 bp, Artificial Sequence
  Synthetic oligonucleotide
aaggaaaacagcaagcgctggaatcccgaa

SEQ ID NO: 180 - DNA, 31 bp, Artificial Sequence
  Synthetic oligonucleotide
gatcctgcagggacccatctgggccaagatc

SEQ ID NO: 181 - DNA, 29 bp, Artificial Sequence
  Synthetic oligonucleotide
gcttggagttttccttcttgagctcccac

SEQ ID NO: 182 - DNA, 31 bp, Artificial Sequence
  Synthetic oligonucleotide
gatcctgcagggtcccatttgggccaagatt

SEQ ID NO: 183 - DNA, 27 bp, Artificial Sequence
  Synthetic oligonucleotide
gtttggaccgctccttctggatctccc

SEQ ID NO: 184 - DNA, 33 bp, Artificial Sequence
  Synthetic oligonucleotide
cccggaaatatcaccaccagcttctcggacgtg

SEQ ID NO: 185 - DNA, 27 bp, Artificial Sequence
  Synthetic oligonucleotide
ctcatcaagaacacaccggtacctgcg

SEQ ID NO: 186 - DNA, 36 bp, Artificial Sequence
  Synthetic oligonucleotide
gcggcaaagtttgctagcttcatcacacagtactcc

SEQ ID NO: 187 - DNA, 66 bp, Artificial Sequence
  Synthetic oligonucleotide
aagaacacaccggtacctgcgaatcctagcattggttatcctcttcctaagtttgctagcttcatc

SEQ ID NO: 188 - DNA, 66 bp, Artificial Sequence
  Synthetic oligonucleotide
gatgaagctagcaaacttaggaagaggataaccaatgctaggattcgcaggtaccggtgtgttctt

SEQ ID NO: 189 - DNA, 66 bp, Artificial Sequence
  Synthetic oligonucleotide
aagaacacaccggtacctgcgaatcctgttaatactgctaatagcactaagtttgctagcttcatc

SEQ ID NO: 190 - DNA, 66 bp, Artificial Sequence
  Synthetic oligonucleotide
gatgaagctagcaaacttagtgctattagcagtattaacaggattcgcaggtaccggtgtgttctt

SEQ ID NO: 191 - DNA, 66 bp, Artificial Sequence
  Synthetic oligonucleotide
aagaacacaccggtacctgcgaatcctcaacctgaacatagcagcactaagtttgctagcttcatc

SEQ ID NO: 192 - DNA, 66 bp, Artificial Sequence
  Synthetic oligonucleotide
gatgaagctagcaaacttagtgctgctatgttcaggttgaggattcgcaggtaccggtgtgttctt

SEQ ID NO: 193 - DNA, 66 bp, Artificial Sequence
  Synthetic oligonucleotide
aagaacacaccggtacctgcgaatcctcgaggagacttcagtgcggcaaagtttgctagcttcatc

SEQ ID NO: 194 - DNA, 66 bp, Artificial Sequence
  Synthetic oligonucleotide
gatgaagctagcaaactttgccgcactgaagtctcctcgaggattcgcaggtaccggtgtgttctt

SEQ ID NO: 195 - DNA, 66 bp, Artificial Sequence
  Synthetic oligonucleotide
aagaacacaccggtacctgcgaatccttcgacccgaggagacgcggcaaagtttgctagcttcatc

SEQ ID NO: 196 - DNA, 66 bp, Artificial Sequence
  Synthetic oligonucleotide
gatgaagctagcaaactttgccgcgtctcctcgggtcgaaggattcgcaggtaccggtgtgttctt

SEQ ID NO: 197 - DNA, 66 bp, Artificial Sequence
  Synthetic oligonucleotide
aagaacacaccggtacctgcgaatccttcgaccaccttccgaggagacaagtttgctagcttcatc

SEQ ID NO: 198 - DNA, 66 bp, Artificial Sequence
  Synthetic oligonucleotide
gatgaagctagcaaacttgtctcctcggaaggtggtcgaaggattcgcaggtaccggtgtgttctt

SEQ ID NO: 199 - DNA, 66 bp, Artificial Sequence
  Synthetic oligonucleotide
aagaacacaccggtacctgcgaatccttcgaccaccttcagtcgaggagactttgctagcttcatc

SEQ ID NO: 200 - DNA, 66 bp, Artificial Sequence
  Synthetic oligonucleotide
gatgaagctagcaaagtctcctcgactgaaggtggtcgaaggattcgcaggtaccggtgtgttctt

SEQ ID NO: 201 - DNA, 66 bp, Artificial Sequence
  Synthetic oligonucleotide
aagaacacaccggtacctgcgaatccttcgggacgaggagactcggcgaagtttgctagcttcatc

SEQ ID NO: 202 - DNA, 66 bp, Artificial Sequence
  Synthetic oligonucleotide
gatgaagctagcaaacttcgccgagtctcctcgtcccgaaggattcgcaggtaccggtgtgttctt

SEQ ID NO: 203 - DNA, 42 bp, Artificial Sequence
  Synthetic oligonucleotide
gcgaatccttcgaccaccggcagtgcggcaaagtttgcttcc

SEQ ID NO: 204 - protein, 6 aa, Artificial Sequence
  Synthetic peptide
ANPSFI

SEQ ID NO: 205 - protein, 16 aa, Artificial Sequence
  Synthetic peptide
ANPGGGGGGGGGGSFI

SEQ ID NO: 206 - protein, 16 aa, Artificial Sequence
  Synthetic peptide
ANPGGGFGGGGGGSFI

SEQ ID NO: 207 - protein, 16 aa, Artificial Sequence
  Synthetic peptide
ANPGGGGGGGKGGSFI

SEQ ID NO: 208 - protein, 16 aa, Artificial Sequence
  Synthetic peptide
ANPGGGGGGGGFGSFI

SEQ ID NO: 209 - protein, 16 aa, Artificial Sequence
  Synthetic peptide
ANPPAEFSATKFASFI

SEQ ID NO: 210 - protein, 16 aa, Artificial Sequence
  Synthetic peptide
ANPPTTFNSQKLNSFI

SEQ ID NO: 211 - protein, 16 aa, Artificial Sequence
  Synthetic peptide
ANPATTFSSTPVNSFI

SEQ ID NO: 212 - protein, 15 aa, Artificial Sequence
  Synthetic peptide
GNITSFSDVPVSSFI

SEQ ID NO: 213 - protein, 16 aa, Artificial Sequence
  Synthetic peptide
ANPRGDFSAAKFASFI

SEQ ID NO: 214 - protein, 16 aa, Artificial Sequence
  Synthetic peptide
ANPSTRGDAAKFASFI

SEQ ID NO: 215 - protein, 16 aa, Artificial Sequence
  Synthetic peptide
ANPSTTFRGDKFASFI

SEQ ID NO: 216 - protein, 16 aa, Artificial Sequence
  Synthetic peptide
ANPSTTFSRGDFASFI

SEQ ID NO: 217 - protein, 16 aa, Artificial Sequence
  Synthetic peptide
ANPVNTANSTKFASFI

SEQ ID NO: 218 - protein, 16 aa, Artificial Sequence
  Synthetic peptide
ANPQPEHSSTKFASFI

SEQ ID NO: 219 - protein, 16 aa, Artificial Sequence
  Synthetic peptide
ANPSIGYPLPKFASFI

SEQ ID NO: 220 - protein, 16 aa, Artificial Sequence
  Synthetic peptide
ANPSGRGDSAKFASFI

SEQ ID NO: 221 - protein, 16 aa, Artificial Sequence
  Synthetic peptide
ANPSTTGSAAKFASFI

SEQ ID NO: 222 - DNA, 2205 bp, adeno-associated virus 2
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctg

SEQ ID NO: 223 - protein, 735 aa, adeno-associated virus 2 [sequence omitted]

SEQ ID NO: 224 - DNA, 7462 bp, Artificial Sequence
  Synthetic polynucleotide
cggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttccaccatcaccatcaccattccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtgtcgcggccgctcgataagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgaggaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccctagagtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttc

SEQ ID NO: 225 - DNA, 2211 bp, adeno-associated virus 9
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 226 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 227 - DNA, 2211 bp, Artificial Sequence
  Synthetic polynucleotide
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttccatcaccaccatcaccattctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 228 - protein, 102 aa, adeno-associated virus 1
GHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIG

SEQ ID NO: 229 - protein, 102 aa, adeno-associated virus 2
GHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIG

SEQ ID NO: 230 - protein, 102 aa, adeno-associated virus 3
GHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIG

SEQ ID NO: 231 - protein, 102 aa, adeno-associated virus 4
GHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIG

SEQ ID NO: 232 - protein, 101 aa, adeno-associated virus 5
AHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIG

SEQ ID NO: 233 - protein, 102 aa, adeno-associated virus 6
GHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIG

SEQ ID NO: 234 - protein, 102 aa, adeno-associated virus 7
GNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIG

SEQ ID NO: 235 - protein, 102 aa, adeno-associated virus 9
GNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIG

SEQ ID NO: 236 - protein, 102 aa, adeno-associated virus 8
GNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIG

SEQ ID NO: 237 - protein, 102 aa, adeno-associated virus 10
GNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIG

SEQ ID NO: 238 - protein, 102 aa, adeno-associated virus 11
GHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIG

SEQ ID NO: 239 - protein, 98 aa, Artificial sequence
  Synthetic polypeptide
GHFHPSPLMGGFGLKHPPPQILIKNTPVPANPPTTFSAKLASFITQYSTGQVSVEIEWELQKENSKRWNPEIQYTSNYKSNVDFAVDTGVYSEPRPIG

SEQ ID NO: 240 - protein, 6 aa, adeno-associated virus 1
SATKFA

SEQ ID NO: 241 - protein, 6 aa, adeno-associated virus 3b
SPAKFA

SEQ ID NO: 242 - protein, 6 aa, adeno-associated virus 4
SSTPVN

SEQ ID NO: 243 - protein, 6 aa, adeno-associated virus 5
SDVPVS

SEQ ID NO: 244 - protein, 6 aa, adeno-associated virus 7
TPAKFA

SEQ ID NO: 245 - protein, 6 aa, adeno-associated virus 8
NQSKLN

SEQ ID NO: 246 - protein, 6 aa, adeno-associated virus 9
NKDKLN

SEQ ID NO: 247 - protein, 6 aa, adeno-associated virus 10
SQAKLA

SEQ ID NO: 248 - protein, 6 aa, adeno-associated virus 11
TAARVD

(19) United States
(12) Patent Application Publication
(10) Pub. No.: US 2009/0215879 A1
(43) Pub. Date: Aug. 27, 2009

(54) METHODS AND COMPOSITIONS FOR ADENO-ASSOCIATED VIRUS (AAV) WITH HI LOOP MUTATIONS

(75) Inventors: NINA DIPRIMIO, CARRBORO, NC (US); RICHARD JUDE SAMULSKI, CHAPEL HILL, NC (US)

Correspondence Address: MYERS BIGEL SIBLEY & SAJOVEC, PO BOX 37428, RALEIGH, NC, 27627, US

(73) Assignee: UNIVERSITY OF NORTH CAROLINA AT CHAPEL HILL

(21) Appl. No.: 12/369,945

(22) Filed: Feb. 12, 2009

Related U.S. Application Data
(60) Provisional application No. 61/031,581, filed on Feb. 26, 2008.

(51) Int. Cl. A61K 31/7088; C07K 14/00; C12N 15/00; C12N 15/87

(52) U.S. Cl. 514/44 R; 530/350; 435/320.1; 435/455

(57) ABSTRACT

The invention provides modified AAV capsid proteins comprising substitutions in the HI loop. Suitable substitutions include affinity tags, sequences that facilitate detection and/or targeting peptides. The invention also provides virus capsids and virus vectors comprising the modified AAV capsid proteins and methods of using the same. Further provided are methods of purifying the modified AAV capsid subunits, virus capsids and virus vectors of the invention.

### STATEMENT OF PRIORITY

This application claims the benefit, under 35 U.S.C. § 119(e), of U.S. Provisional Application No. 61/031,581, filed Feb. 26, 2008, the entire contents of which are incorporated by reference herein.

### STATEMENT OF GOVERNMENT SUPPORT

Aspects of this invention were supported by federal funding provided under Grant Nos. 2-T32-GM007040, P01 HL051818, P01 HL594412 and P01 HL51811 from the National Institutes of Health. The U.S. Government has certain rights in this invention.

### FIELD OF THE INVENTION

The present invention relates to adeno-associated virus (AAV) capsid proteins with HI loop mutations, as well virus capsids and virus vectors comprising the mutated AAV capsid proteins, and methods of using the mutated AAV capsids and vectors of this invention.

### BACKGROUND OF THE INVENTION

Adeno-associated virus (AAV), a 26 nm nonpathogenic human parvovirus, is distinct from most viruses due to the dependence on a helper virus for productive infection (adenovirus or herpes simplex virus) (Berns and Linden. (1995) Bioessays 17:237-245). In light of the rapidly growing applications of AAV as a gene therapy vector (Warrington et al. (2006) Hum Genet 119:571-603; Wu et al. (2006) Mol Ther 14:316-327), several efforts to understand events in the infectious pathway including host cell recognition (Akache et al. (2006) J Virol 80:9831-9836; Di Pasquale et al. (2003) Nat Med 9:1306-1312; Kern et al. (2003) J Virol 77:11072-11081; Qing et al. (1999) Nat Med 5:71-77; Walters et al. (2001) J Biol Chem 276:20610-20616), intracellular trafficking (Bartlett et al (2000) J Virol 74:2777-2785; Ding et al. (2005) Gene Ther 12:873-880) and uncoating (Thomas et al. (2004) J Virol 78:3110-3122) in the absence of helper are currently underway. Further, the crystal structures of several AAV serotypes (Padron et al. (2005) J Virol 79:5047-5058; Walters et al. (2004) J Virol 78:3361-3371; Xie et al. (2003) Acta Crystallogr D Biol Crystallogr 59:959-9570) and related parvoviruses (Agbandje-McKenna et al. (1998) Structure 6:1369-1381; Kaufmann et al. (2004) Proc Natl Acad Sci USA 101:11628-11633) have been determined over the past few years.

With respect to AAV, the capsid is encoded by three overlapping viral proteins (VPs) VP1, VP2 and VP3 (Rose et al. (1971) J Virol 8:766-770), which are incorporated into a 60 subunit capsid in a 1:1:10 ratio. VP1 has a unique N-terminus containing a phospholipase (PLA2) domain (Girod et al. (2002) J Gen Virol 83:973-978) and nuclear localization sequences (Grieger et al. (2006) J Virol 80:5199-5210; Sonntag et al. (2006) J Virol 80:11040-11054) thought to be necessary for endosomal escape (Farr et al. (2005) Proc Natl Acad Sci USA 102:17148-17153) and possibly nuclear entry (Vihinen-Ranta et al. (2002) J Virol 76:1884-1891). VP2 also has an extended N-terminus (compared to VP3) that remains internal to the capsid similar to VP1 until exposed to experimental conditions involving low pH or heat (Kronenberg et al. (2005) J Virol 79:5296-5303). Although this protein has been suggested to be nonessential for viral assembly and infectivity (Warrington et al. (2004) J Virol 78:6595-6609) its exact role remains unknown (Grieger et al. (2006) J Virol 80:5199-5210). VP3 is the primary capsid protein (contained within VP1 and VP2) that constitutes the surface topology of the AAV capsid, which in turn dictates antigenicity (Herzog (2007) Mol Ther 15:649-650: Lochrie et al. (2006) J Virol 80:821-834) and tropism (Akache et al. (2006) J Virol 80:9831-9836; Asokan et al. (2006) J Virol 80:8961-8969; Opie et al. (2003) J Virol 77:6995-7006). Based on crystal structures of AAV, the VP amino acids involved in forming the icosahedral five-fold (FIG. 1B), three-fold (Asokan et al. (2006) J Virol 80:8961-8969) and two-fold symmetry interfaces have been visualized. The three-fold axis has the largest amount of buried surface area and the highest contact energy, being the most interdigitated region of the capsid (Xie et al. (2003) Acta Crystallogr D Biol Crystallogr 59:959-9570). The surface loops at the three-fold axis of symmetry are thought to be involved in host cell receptor binding (Asokan et al. (2006) J Virol 80:8961-8969; Kern et al. (2003) J Virol 77:11072-11081) and has been the target of several mutagenesis studies (Lochrie et al. (2006) J Virol 80:821-834; Opie et al. (2003) J Virol 77:6995-7006; Shi et al. (2006) Hum Gene Ther 17:353-361; Wu et al. (2000) J Virol 74:8635-8647; Wu et al. (2006) J Virol 80:11393-11397). In addition, recent data has shown that a single amino acid change (K531E) located at the base of the three-fold loops has the ability to alter the phenotypes of multiple AAV serotypes (Wu et al. (2006) J Virol 80:11393-11397), suggesting an incomplete understanding of this critical region. The two-fold axis of symmetry has the weakest amino acid interactions and the lowest contact energy, while the five-fold symmetry axis is thought to have intermediate interactions (Xie et al. (2003) Acta Crystallogr D Biol Crystallogr 59:959-9570).

The pentameric assembly of VP3 subunits results in the formation of twelve pores at the five-fold axis of symmetry (FIG. 1B), which have been the focus of several recent investigations. Mutagenesis of residues that constitute the pore has suggested a role in assembly and packaging (Bleker et al. (2005) J Virol 79:2528-3540; Grieger et al. (2007) J Virol 81:7833-7843; Wu et al. (2000) J Virol 74:8635-8647). Therefore, it is likely that the five-fold pore is involved in for DNA packaging including Rep protein binding, capsid assembly, and VP1 N-terminus exposure. Surrounding this pore at the five-fold axis of symmetry is a prominent region of the AAV capsid—the HI loop located between β strands βH and βI, which spans residues 653 to 669 (VP1 numbering) and extends to overlap each adjacent subunit (FIG. 1). Recent data have shown that the HI loop conformationally changes upon capsid interaction with the primary receptor heparan sulfate proteoglycan, alluding to an important capsid conformational change for subsequent stages in the AAV life cycle.

### SUMMARY OF THE INVENTION

The present invention provides modified adeno-associated virus (AAV) capsid proteins, in which amino acids are substituted in the HI loop. Nonlimiting examples of suitable substitutions include affinity tags, sequences that facilitate detection, and/or targeting peptides (e.g., a poly-histidine tag, a streptavidin affinity peptide, a receptor ligand, and the like). In some embodiments, the modified AAV capsid proteins of this invention comprise an RGD domain as a substitution and can be used for targeting or purifying the virus via integrin receptor recognition.

In representative embodiments, the present invention provides a universal purification method applicable to any AAV capsid protein or a virus capsid or virus vector comprising the same, for example, by substituting an affinity tag (e.g., two or more histidine residues or a streptavidin affinity peptide) into the HI loop of the AAV capsid protein.

Accordingly, as one aspect, the invention provides an AAV capsid protein comprising one or more amino acid substitutions in the HI loop of the AAV capsid protein, for example, in the region of amino acid positions 658 through 667 of the native AAV2 capsid protein or the corresponding positions of the capsid subunit of another AAV.

As a further aspect, the invention provides a virus capsid or virus vector comprising an AAV capsid protein of this invention. In particular embodiments, the virus vector or virus capsid comprises 12, 30 or 60 copies of the modified AAV capsid protein.

As another aspect, the invention provides a pharmaceutical formulation comprising a virus capsid and/or virus vector of this invention in a pharmaceutically acceptable carrier.

Still further, the invention provides a method of administering a nucleic acid to a cell comprising contacting the cell with a virus vector or pharmaceutical formulation of this invention.

As yet another aspect, the invention provides a method of delivering a nucleic acid to a subject comprising administering to the subject a virus vector or pharmaceutical formulation of this invention.

As a further aspect, the invention provides a method of modulating the tissue tropism of a virus vector in a subject comprising administering to the subject a virus vector or pharmaceutical formulation of the invention.

The invention also contemplates purification methods and as another aspect provides a method of purifying an AAV capsid protein comprising one or more histidine residues or a virus capsid or virus vector comprising the same from a sample, the method comprising:
(a) providing a solid support comprising a matrix, wherein the matrix comprises nickel;
(b) contacting the solid support with a sample comprising the AAV capsid protein, virus capsid and/or virus vector of this invention; and
(c) eluting the bound AAV capsid protein, virus capsid and/or virus vector from the matrix.

These and other aspects of the invention will be set forth in more detail in the description of the invention that follows.

### BRIEF DESCRIPTION OF THE DRAWINGS

FIGS. 1A-B. HI loop comparison between various AAV serotypes and autonomous parvoviruses. (A) Comparison of amino acid sequence homologies between representative AAV serotypes. The HI loop amino acid sequence alignment is shown on the right. Boxed are the ten most variable amino acids. (B) An arrow indicates the HI loop on an AAV2 pentamer, which extends from one VP subunit and overlaps the neighboring VP.

FIGS. 2A-B. AAV2 HI loop deletion and glycine substitution characterizations. (A) AAV2 HI loop sequence alignment showing amino acids 658-667 that were removed (AAV2 HI−/−) or substituted with glycine residues (AAV2 poly-glycine). (B) Western dot blot analysis of CsCl gradient fractions from AAV2, AAV2 HI−/− and AAV2 poly-glycine preparations with A20 antibody. Gradient fractions were collected, and blotted onto a nitrocellulose membrane. The membrane was incubated with primary antibody A20 (1:20) and incubated with horseradish peroxidase conjugated secondary goat anti-mouse (1:5000).

FIGS. 3A-C. AAV2 HI1 and AAV2 HI8 substitution mutant characterization. (A) Sequence alignment of AAV2, AAV1 and AAV8 HI loop amino acids. (B) Radioactive DNA dot blot shown as fold change in titer (left panel) and luciferase assay on infected 293 cells with 3000 vg/cell (right panel n=3 SD=black bars). (C) Heparin binding profiles of AAV2, AAV2 HI1 and AAV2 HI8. 500 ul of heparin type IIIS conjugated agarose beads (Sigma) were incubated with 1E10 vector genome containing particles (L=load) for 10 mins at room temperature. The washes (1×PBS) and elutions (0.2M-0.6M PBS) were collected and capsids were detected via western dot blot analysis with A20 monoclonal antibody (1:20).

FIGS. 4A-B. AAV2 HI4 substitution mutant characterization. (A) AAV2 HI4 titer and transduction. AAV2 and AAV2 HI4 titers were quantified via radioactive DNA dot blot against the luciferase transgene, shown as fold change in titer (left panel). AAV2 HI4 transduction was quantified via luciferase assay of 293 cells infected with 3000 vg/cell (right panel n=3 SD=black bars). (B) Heat treatment of 6E8 AAV2 and AAV2 HI4 viral DNA containing particles, with temperatures tested on the left hand side were transferred to a nitrocellulose membrane and blotted with the antibodies listed across the bottom at a ratio of 1:20.

FIGS. 5A-B. AAV2 HI5 substitution mutant characterization. (A) A sequence alignment of AAV2 and AAV5 HI loop residues. The amino acid position deleted in AAV5 relative to AAV2 in this region is depicted as a dash in the alignment. (B) Western dot blot analysis of AAV2, AAV2 HI5, and AAV2 HI5 TTSF (threonine inserted at amino acid position 659) CsCl gradient fractions blotted with A20 antibody (1:20).

FIGS. 6A-C. AAV2 HI loop peptide substitution mutant characterizations. (A) A sequence alignment shows residues in the AAV2 HI loop that were substituted with specific peptides indicated in gray. (B) HI loop peptide substitution titer and transduction. Fold change in titer as compared to AAV2 determined by radioactive DNA dot blot analysis (left panel). Fold change in transduction of infected 293 cells (3000 vg/cell) determined by luciferase assay (right panel n=3 SD=black bars). (C) Western blot analysis of peptide substitution mutants incubated overnight at 4° C. with A1 (1:20) and B1 (1:20) monoclonal antibodies. A red arrow indicates an additional protein band detected with A1 antibody (bottom panel).

FIGS. 7A-C. Residue F661 structure model and sequence alignment and AAV2 F661G substitution mutant characterization. (A) Sequence alignment of representative serotypes shows that F661 is conserved (gray). (B) Fold change in AAV2 F661 G viral titer determined by radioactive DNA dot blot against the luciferase transgene. (C) Transduction quantified post 293 cell infection with AAV2 and AAV2 F661G (3000 vg/cell) and evaluated via luciferase assay (n=3 SD=black bars).

FIG. 8. AAV2 F661G VP1 externalization and virus infectivity. Heat treatment of 6E8 AAV2 and AAV2 F661 G viral DNA containing particles (temperatures across the bottom). Treated capsids were blotted on a nitrocellulose membrane and intact capsids, dissociated VPs and externalized VP1 unique N-termini were detected with A20, B1 and A1 antibodies (1:20) as indicated on the right hand side.

FIG. 9. AAV2 F661G viral protein incorporation. Western blot analysis of AAV2 and AAV2 F661G capsids with B1 and A1 antibodies (1:20), listed below the blot, detected an additional protein band at ˜77 kDa (red arrows).

FIGS. 10 A-B. Substitution of the AAV2 HI loop with a hexa-histidine motif. (A) AAV2 HI6× His titer was determined via qPCR of the luciferase transgene and compared to AAV2 wildtype titer. (B) AAV2 and AAV2 HI6× His transduction was quantified via luciferase assay of 293 cells infected with 3000 vector genomes per cell (n=3 SD=black bars).

FIGS. 11A-B. Affinity chromatography purification of AAV capsids containing the hexa-histidine motif. (A) AAV2 HI6× His (left) and AAV9 HI6× His (right) was purified via metal affinity chromatography through a 1 ml His-Trap HP nickel column (Amersham). At a wavelength of 280 nm the FPLC detector determined in which fraction the protein eluted from the nickel column. (B) Vector genome quantification of AAV2 HI6× His and AAV9 HI6× His nickel column fractions as compared to AAV2. Approximately 1E13 vector genome containing particles were loaded into the injection loop and injected across the nickel column. Total vector genomes in each column fraction were quantified via qPCR of the luciferase transgene.

FIGS. 12A-C. Vector purity and gold particle labeling. (A) Silver stain analysis of AAV2 HI6× His and AAV9 HI6× His nickel column fractions. 30 ul of the load (L), flowthrough (FT), wash (W) and elution (E) fractions were loaded onto a NuPage gel from Invitrogen. Protein was detected via Silver Express (Invitrogen), (B) EM analysis of viral particle purity post nickel column purification. 15 ul of AAV2 HI6× His (left) and AAV9 HI6× His (right) load and peak elution fractions were incubated on glow discharged copper grids. Grids were washed with 25 ul ddH2O and incubated with 2% uranyl acetate negative stain. (C) EM analysis of Ni-NTA nanogold particle labeled AAV2 HI6× His capsids. AAV2 (left) and AAV2 HI6× His (right) were incubated with Ni-NTA nanogold particles (Nanoprobes). Ni-NTA nanogold was present in excess to the total number of histidine tags in the sample.

FIGS. 13A-B. Hexa-histidine vectors detarget the liver. (A) 1E10 vector genome containing AAV2 and AAV2 HI6× His were injected intramuscularly (B) intravenously. Two weeks post vector administration mice were injected IP with D-luciferin firefly luciferase substrate (Nanolight) and imaged for 1 minute or 5 minutes, respectively via an IVIS Xenogen imaging system 5 minutes post substrate administration.

FIGS. 14A-B. Chimeric hexa-histidine vectors rescue tissue transduction in vivo. (A) 1E12 vector genome containing AAV2 1:AAV2 HI6× His 1 and AAV2 4:AAV2 HI6× His 1 chimeras were passed through the FPLC nickel binding column. Flowthrough (FT), wash (W), and elution (E) column fractions were collected and vector genomes in each fraction were quantified via qPCR of the luciferase transgene. (B) Photons emitted post IM and IV injection were quantified and graphed (left panel). N=3 or 4 and SD=black bars for IM injections and N=3 and SD=black bars for IV injections. Liver tissue was harvested 2 weeks post IV injection and vector genomes present in the liver post IV injection were quantified via qPCR with primers against the luciferase transgene (right panel). The m-lam gene present in the sample was quantified as a control. N=3 and SD=black bars.

### DETAILED DESCRIPTION OF THE INVENTION

The present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

The designation of all amino acid positions in the AAV capsid proteins in the description of the invention and the appended claims is with respect to VP1 capsid subunit numbering (GenBank Accession No. AAC03780). It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3).

References to a particular range amino acid positions within the AAV capsid protein are intended to be inclusive unless stated otherwise. For example, the amino acid positions “662 to 667” is intended to be inclusive of amino acids 662, 663, 664, 665, 666 and 667.

Except as otherwise indicated, standard methods known to those skilled in the art may be used for the construction of rAAV constructs, modified capsid proteins, packaging vectors expressing the AAV rep and/or cap sequences, and transiently and stably transfected packaging cells. Such techniques are known to those skilled in the art. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

### DEFINITIONS

The following terms are used in the description herein and the appended

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

Moreover, the present invention also contemplates that in some embodiments of the invention, any feature or combination of features set forth herein can be excluded or omitted.

As used herein, the terms “reduce,” “reduces,” “reduction” and similar terms mean a decrease of at least about 25%, 35%, 50%, 75%, 80%, 85%, 90%, 95%, 97% or more.

As used herein, the terms “enhance,” “enhances,” “enhancement” and similar terms indicate an increase of at least about 25%, 50%, 75%, 100%, 150%, 200%, 300%, 400%, 500% or more.

The term “parvovirus” as used herein encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of relatively new AAV serotypes and clades have been identified (see, e.g., Gao et al., (2004) J. Virology 78:6381-6388; Moris et al., (2004) Virology 33:375-383; and Table 1).

The genomic sequences of various serotypes of AAV and the autonomous parvoviruses, as well as the sequences of the native terminal repeats (TRs), Rep proteins, and capsid subunits are known in the art. Such sequences may be found in the literature or in public databases such as GenBank. See, e.g., GenBank Accession Numbers NC_{—}002077, NC_{—}001401, NC_{—}001729, NC_{—}001863, NC_{—}001829, NC_{—}001862, NC_{—}000883, NC_{—}001701, NC_{—}001510, NC_{—}006152, NC_{—}006261, AF063497, U89790, AF043303, AF028705, AF028704, J02275, J01901, J02275, X01457, AF288061, AH009962, AY028226, AY028223, NC_{—}001358, NC_{—}001540, AF513851, AF513852, AY530579; the disclosures of which are incorporated by reference herein for teaching parvovirus and AAV nucleic acid and amino acid sequences. See also, e.g., Srivistava et al., (1983) J. Virology 45:555; Chiorini et al., (1998) J. Virology 71:6823; Chiorini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Virol. 58:921; Gao et al., (2002) Proc. Nat. Acad. Sci. USA 99:11854; Moris et al., (2004) Virology 33:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1.

The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV4 (Padron et al., (2005) J. Virol. 79: 5047-58), AAV5 (Walters et al., (2004) J. Virol. 78: 3361-71) and CPV (Xie et al., (1996) J. Mol. Biol. 6:497-520 and Tsao et al., (1991) Science 251: 1456-64).

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest. Those skilled in the art will appreciate that transcription of a heterologous nucleic acid sequence from the viral genome may not be initiated in the absence of trans-acting factors, e.g., for an inducible promoter or otherwise regulated nucleic acid sequence. In the case of a rAAV genome, gene expression from the viral genome may be from a stably integrated provirus, from a non-integrated episome, as well as any other form in which the virus may take within the cell.

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotide), but in representative embodiments are either single or double stranded DNA sequences.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide.

As used herein, by “isolate” or “purify” (or grammatical equivalents) a capsid protein, virus capsid or virus vector, it is meant that the capsid protein, virus capsid or virus vector is at least partially separated from at least some of the other components in the starting material. In particular embodiments, the final product is at least about 25%, 35%, 50%, 60%, 75%, 80%, 85%, 90%, 95%, 97%, 98% or 99% pure (w/w %).

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., anti-cancer effects or improvement in transplant survivability.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

The terms “heterologous nucleotide sequence” and “heterologous nucleic acid” are used interchangeably herein and refer to a sequence that is not naturally occurring in the virus. Generally, the heterologous nucleic acid comprises an open reading frame that encodes a polypeptide or nontranslated RNA of interest (e.g., for delivery to a cell or subject).

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

A “rAAV vector genome” or “rAAV genome” is an AAV genome (i.e., vDNA) that comprises one or more heterologous nucleic acid sequences. rAAV vectors generally require only the 145 base terminal repeat(s) (TR(s)) in cis to generate virus. All other viral sequences are dispensable and may be supplied in trans (Muzyczka, (1992) Curr. Topics Microbiol. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one TR sequence (e.g., AAV TR sequence), optionally two TRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or the SV40 hairpin that serves as the origin of SV40 replication can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR” may be from any AAV, including but not limited to serotypes 1, 2, 3, 4, 5, 6, 7, 8, 9, 10 or 11 or any other AAV now known or later discovered (see, e.g., Table 3). An AAV terminal repeat need not have the native terminal repeat sequence (e.g., a native AAV TR sequence may be altered by insertion, deletion, truncation and/or missense mutations), as long as the terminal repeat mediates the desired functions, e.g., replication, virus packaging, integration, and/or provirus rescue, and the like.

The virus vectors of the invention can further be a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can also be duplexed parvovirus particles as described in international patent publication WO 01/92551. Thus, in some embodiments, double stranded (duplex) genomes can be packaged into the virus capsids of the invention.

Further, the viral capsid or genomic elements can contain any other modification (including insertions, deletions and/or substitutions) now known or later identified.

For example, the AAV capsid proteins, virus capsids and virus vectors of the invention can be chimeric in that they and can comprise all or a portion of a capsid subunit from another virus, optionally another parvovirus or AAV, e.g., as described in international patent publication WO 00/28004,

The AAV capsid proteins, virus capsids and virus vectors of the invention can comprise a targeting sequence other than the modifications of the present invention, where the targeting sequence directs interaction with a cell-surface molecule present on a desired target tissue(s) (see, e.g., international patent publication WO 00/28004 and Hauck et al., (2003) J. Virology 77:2768-2774); Shi et al., Human Gene Therapy 17:353-361 (2006) [describing insertion of the integrin receptor binding motif RGD at positions 520 and/or 584 of the AAV capsid subunit]; and U.S. Pat. No. 7,314,912 [describing insertion of the P1 peptide containing an RGD motif following amino acid positions 447, 534, 573 and 587 of the AAV2 capsid subunit]). Other positions within the AAV capsid subunit that tolerate insertions are known in the art including positions 449 and 588 (see, e.g., Grifman et al., Molecular Therapy 3:964-975 (2001)) and position 485.

As another option, the capsid protein or capsid of the invention can comprise a mutation as described in WO 2006/066066. For example, the AAV capsid protein, virus capsid or virus vector can comprise a selective amino acid insertion directly following amino acid position 264 of the AAV2 capsid protein or a corresponding change in the capsid protein from another AAV. By “directly following amino acid position X” it is intended that the insertion immediately follows the indicated amino acid position (for example, “following amino acid position 264” indicates a point insertion at position 265 or a larger insertion, e.g., from positions 265 to 268, etc.).

In other representative embodiments, the modified capsid protein, virus capsid or virus vector of the invention further comprises one or more mutations as described in WO 2007/089632 (e.g., an E→K mutation at amino acid position 531 of the AAV2 capsid protein or the corresponding position of the capsid protein from another AAV).

In further embodiments, the modified capsid protein, virus capsid or vector can comprise an inner loop mutation as described in the United States provisional application filed Feb. 11, 2009 by Asokan et al. entitled “Modified Virus Vectors and Methods of Making and Using the Same.” For example, in particular embodiments, the modified capsid protein, virus capsid or virus vector can comprise a modification at amino acids positions 585 to 590 of the native AAV2 capsid protein or the corresponding positions of another AAV.

### Modified AAV Capsid Proteins and Virus Capsids and Virus Vectors Comprising the Same.

The present invention provides modified AAV capsid proteins and virus capsids and virus vectors comprising the same. The modified AAV capsid proteins of the invention comprise a substitution and/or insertion in the HI loop. Nonlimiting examples of suitable sequences that can be substituted and/or inserted into the HI loop include affinity purification tags (e.g., two or more histidine residues or a streptavidin affinity peptide), sequences that facilitate detection (e.g., that can be used to tag the capsid protein with gold nanoparticles) and targeting sequences (e.g., receptor ligands and peptides that interact with the extracellular matrix).

As one aspect, the invention provides a capsid protein comprising one or more amino acid substitutions in the HI loop. In representative embodiments, the capsid protein comprises 1, 2, 3, 4, 5, 6, 7, 8, 9, 10, 12, 14 or more amino acid substitutions in the HI loop, for example, 2 to 4, 6, 7, 8, 10, 12 or 14 amino acid substitutions, 3 to 4, 5, 6, 7, 8, 10, 12 or 14 amino acid substitutions, or 4 to 5, 6, 7, 8, 10 or 12 amino acid substitutions. In particular embodiments, the amino acid substitution(s) is made in the region of amino acid positions 649 through 678 of the native AAV2 capsid protein (or any subset of amino acids therein) or the corresponding region of the capsid protein from another AAV. Optionally, the amino acid substitution(s) is made in the region of amino acid positions 649 through 667 (VP1 numbering), amino acid positions 658 through 678, amino acid positions 658 through 670, amino acid positions 665 through 667, amino acid positions 662 through 670, or amino acid positions 662 through 667 of the native AAV2 capsid protein or the corresponding region of the capsid protein from another AAV.

In embodiments of the invention, substitutions are made at one or more amino acid positions in the variable region of the HI loop, for example, at amino acid positions 658, 659, 660, 661, 662, 663, 664, 665, 666 and/or 667 of the native AAV2 capsid protein or the corresponding position(s) of the capsid protein of another AAV. In representative embodiments, amino acid position 661 of the native AAV2 capsid protein or the corresponding position of the capsid protein of another AAV is not substituted and/or a conservative substitution that conserves hydrophobic interactions is made (e.g., the substitution is with a W, T or H residue).

Thus, in representative embodiments, the invention contemplates an AAV capsid protein comprising one or more amino acid substitutions in the HI loop of the AAV capsid protein, wherein the amino acid substitution is in the region of amino acid positions 658 through 667 of the native AAV2 capsid protein or the corresponding positions of the capsid subunit of another AAV. Optionally, the amino acid substitution is in the region of amino acid positions 662 through 667 of the native AAV2 capsid protein or the corresponding positions of the capsid subunit of another AAV.

An alignment of the HI loop region of a variety of AAV serotypes is shown in Table 5. Thus, the amino acids “corresponding” to amino acid positions 662 through 670 or any other position of the HI loop of the native AAV2 capsid protein can be readily determined for the other AAV serotypes shown in Table 5 or any other AAV now known or later discovered, including AAAV having non-naturally occurring capsid sequences.

The modifications to the AAV capsid protein according to the present invention are “selective” modifications. This approach is in contrast to previous work with whole subunit or large domain swaps between AAV serotypes (see, e.g., international patent publication WO 00/28004 and Hauck et al., (2003) J. Virology 77:2768-2774). In particular embodiments, a “selective” modification results in the substitution of less than about 20, 15, 12, 10, 9, 8, 7, 6, 5, 4 or 3 contiguous amino acids.

The present invention can advantageously be practiced to provide a convenient purification scheme for the modified AAV capsid proteins and for virus capsids and virus vectors comprising the same. As a non-limiting example, one or more histidine residues can be substituted into an AAV capsid protein (substitutions are as described above), for example, at amino acid positions 662, 663, 664, 665, 666, and/or 667 of the native AAV2 capsid protein or the corresponding position(s) of the capsid protein of another AAV. It is well-known in the art that poly-histidine tags can be used to purify recombinant proteins on the basis of affinity for nickel. A poly-histidine tag according to the present invention can comprise 3, 4, 5, 6, 7, 8, 9 and/or 10 histidine residues (optionally contiguous histidine residues), at least 4 histidine residues (for example, 4-10 histidine residues or 4-9 histidine residues), at least 5 histidine residues (for example, 5-9 histidine residues or 5-8 histidine residues), at least 6 histidine residues (for example, 6-7 histidine residues), or 6 histidine residues (i.e., hexa-His). In particular embodiments, 3, 4, 5 or all 6 of amino acid positions 662 through 667 (optionally contiguous amino acid positions) of the native AAV2 capsid protein or the corresponding position(s) of the capsid protein of another AAV is substituted with histidine residues. To illustrate, 3, 4, 5, or all 6 of amino acid positions 663 through 668 (optionally contiguous amino acid positions) of the native AAV9 capsid protein can be substituted with histidine residues.

In representative embodiments, the AAV capsid protein has the amino acid sequence of the AAV2 HI6× His capsid protein (SEQ ID NO:1) or the AAV9 HI6× His capsid protein (SEQ ID NO:2) shown in Table 4.

In particular embodiments, the modified capsid protein comprising a poly-histidine substitution in the HI loop has enhanced binding affinity to nickel (e.g., a nickel column) as compared with a suitable control AAV vector that does not comprise said histidine substitution. Accordingly, the modified AAV capsid subunit (or a virus capsid or virus vector comprising the same) can be purified using nickel affinity purification. The modified AAV capsid protein comprising the histidine substitution can also have enhanced affinity for (e.g., chelate) other metal ions (e.g., iron) as compared with a suitable control AAV vector that does not comprise the histidine substitution. For example, a modified AAV capsid protein comprising the histidine substitution and virus capsids and virus vectors incorporating the same can chelate iron and be purified using magnetic based approaches (e.g., magnetic beads).

In general the compositions and methods of this invention can be employed to use any binding pair for detection and/or purification of virus capsids and virus vectors of this invention. For example, one member of a binding pair is inserted into, substituted into and/or tethered into the HI loop and the other member of the binding pair is used for affinity purification and/or detection according to standard methods well known in the art.

Further, the histidine residues substituted into the HI loop as described herein can be tagged with gold nanoparticles, which are useful, for example, for electron microscopy. The capsid protein can be tagged with gold nanoparticles by any method known in the art, for example, using Ni-NTA (nitrolotriacetic acid).

Moreover, nickel conjugates with detectable markers such as alkaline phosphatase, horseradish peroxidase or a fluorophore are commercially available and can be used to detect a modified AAV capsid protein of the invention comprising a histidine substation or a virus capsid or virus vector comprising the same (for example, in western blot analysis).

The present invention can also be practiced to facilitate purification of the modified AAV capsid proteins and virus capsids and virus vectors comprising the same with streptavidin (i.e., by substitution of a streptavidin affinity peptide into the HI loop). According to this aspect of the invention, a streptavidin affinity peptide can be substituted into the HI loop as described above. Nonlimiting examples of streptavidin affinity peptides include EPDW, AWRHPQGG and GDWVFI. Other streptavidin affinity peptides are known in the art. For example, U.S. Pat. No. 5,506,121 describes streptavidin affinity peptides including peptides having the general sequence Trp-X-His-Pro-Gln-Phe-Y-Z, wherein X represents any amino acid residue, and Y and Z both represent Gly or where Y represents Glu, Z represents Arg or Lys. A streptavidin affinity peptide WSHPQFEK sold under the name STREP TAG II® is also suitable for substitution in the HI loop.

The modified AAV capsid proteins of the invention can also comprise any sequence that facilitates detection (e.g., visualization) or purification of the modified capsid protein or a modified virus capsid or virus vector comprising the same. For example, a peptide having antigenic properties can be incorporated into the HI loop to facilitate purification by immunopurification techniques. As a non-limiting illustration, a FLAG motif can be substituted into the HI loop (substitutions are as described above), and detected with commercially available antibodies (Eastman-Kodak, Rochester, N.Y.). A detectable capsid subunit, virus capsid and virus vector finds use, e.g., for detecting the presence and/or persistence of the capsid protein, virus capsid or virus vector in a cell, tissue or subject as well as in laboratory techniques to detect and/or quantify the presence of the capsid protein, virus capsid or virus vector.

As is known in the art, RGD peptides can be used for purification and/or targeting via binding to integrin receptors. In particular embodiments of the invention, an RGD sequence is substituted into the HI loop. In exemplary embodiments, the RGD sequence is substituted at amino acids 658 to 660, amino acids 660 to 662, or amino acids 662 to 664 of the native AAV2 capsid protein or the corresponding position of another AAV.

Dipeptide libraries are known in the art and a dipeptide can be incorporated into the HI loop of the modified AAV capsid protein. It is known that such dipeptide libraries can be selected for those dipeptides that confer the ability to bind to a matrix, which property can be used to detect and/or purify the modified AAV capsid protein or a virus capsid or vector comprising the same.

As another possibility, one or more non-naturally occurring amino acids as described by Wang et al., Annu Rev Biophys Biomol Struct. 35:225-49 (2006)) can be incorporated into the AAV capsid protein in the HI loop to facilitate detection and/or purification of the modified AAV capsid protein, virus capsid and/or virus vector. These unnatural amino acids can advantageously be used to chemically link molecules of interest to the AAV capsid protein that can be utilized for affinity purification (including immunopurification) techniques and/or to detect the protein. Such molecules include receptor ligands, receptors, a binding peptide (e.g., a streptavidin affinity peptide), an antibody or antibody fragment, biotin, detectable enzymes (e.g., alkaline phosphatase, horseradish peroxidase), fluorophores, chromophores, glycans, RNA aptamers, and the like. Methods of chemically modifying amino acids are known in the art (see, e.g., Greg T. Hermanson, Bioconjugate Techniques, 1^{st} edition, Academic Press, 1996).

The invention also contemplates that a targeting sequence can be substituted into the HI loop (substitutions are as described hereinabove), e.g., to direct the tropism of the virus to a desired target tissue(s). Any suitable targeting sequence can be incorporated into the HI loop of the AAV capsid protein. Alternatively or additionally, a targeting sequence can be added at an orthogonal position (outside of the HI loop) to target the vector. For example, in embodiments of the invention, virus capsids and virus vectors comprising the modified AAV capsid proteins are detargeted from the liver. According to this embodiment, a targeting sequence can be incorporated into any suitable site (e.g., in the HI loop as described herein and/or any other suitable site of the modified AAV capsid protein) of a modified AAV capsid protein to target the virus capsid or virus vector to a desired target tissue(s), and optionally confer selective transduction for particular tissue(s).

In representative embodiments, the targeting sequence may be a virus capsid sequence (e.g., an autonomous parvovirus capsid sequence, AAV capsid sequence, or any other viral capsid sequence) that directs infection to a particular cell type(s).

As another nonlimiting example, the respiratory syncytial virus heparin binding domain may be inserted or substituted into a capsid subunit that does not typically bind HS receptors (e.g., AAV 4, AAV5) to confer heparin binding to the resulting mutant.

B19 infects primary erythroid progenitor cells using globoside as its receptor (Brown et al., (1993) Science 262:114). The structure of B19 has been determined to 8 Å resolution (Agbandje-McKenna et al., (1994) Virology 203:106). The region of the B19 capsid that binds to globoside has been mapped between amino acids 399-406 (Chapman et al., (1993) Virology 194:419), a looped out region between β-barrel structures E and F (Chipman et al., (1996) Proc. Nat. Acad. Sci. USA 93:7502). Accordingly, the globoside receptor binding domain of the B19 capsid may be substituted into the AAV capsid protein to target a virus capsid or virus vector comprising the same to erythroid cells.

The exogenous targeting sequence may be any amino acid sequence encoding a peptide that alters the tropism of a virus capsid or virus vector comprising the modified AAV capsid protein. In particular embodiments, the targeting peptide or protein may be naturally occurring or, alternately, completely or partially synthetic. Exemplary targeting sequences include ligands and other peptides that bind to cell surface receptors and glycoproteins, such as RGD peptide sequences, bradykinin, hormones, peptide growth factors (e.g., epidermal growth factor, nerve growth factor, fibroblast growth factor, platelet-derived growth factor, insulin-like growth factors I and II, etc.), cytokines, melanocyte stimulating hormone (e.g., α, β or γ), neuropeptides and endorphins, and the like, and fragments thereof that retain the ability to target cells to their cognate receptors. Other illustrative peptides and proteins include substance P, keratinocyte growth factor, neuropeptide Y, gastrin releasing peptide, interleukin 2, hen egg white lysozyme, erythropoietin, gonadoliberin, corticostatin, β-endorphin, leu-enkephalin, rimorphin, α-neo-enkephalin, angiotensin, pneumadin, vasoactive intestinal peptide, neurotensin, motilin, and fragments thereof as described above. As yet a further alternative, the binding domain from a toxin (e.g., tetanus toxin or snake toxins, such as α-bungarotoxin, and the like) can be substituted into modified AAV capsid protein as a targeting sequence. In a yet further representative embodiment, the AAV capsid protein can be modified by substitution of a “nonclassical” import/export signal peptide (e.g., fibroblast growth factor-1 and -2, interleukin 1, HIV-1 Tat protein, herpes virus VP22 protein, and the like) as described by Cleves (Current Biology 7:R318 (1997)) into the AAV capsid protein. Also encompassed are peptide motifs that direct uptake by specific cells, e.g., a FVFLP peptide motif triggers uptake by liver cells.

Phage display techniques, as well as other techniques known in the art, may be used to identify peptides that recognize any cell type of interest.

The targeting sequence may encode any peptide that targets to a cell surface binding site, including receptors (e.g., protein, carbohydrate, glycoprotein or proteoglycan). Examples of cell surface binding sites include, but are not limited to, heparan sulfate, chondroitin sulfate, and other glycosaminoglycans, sialic acid moieties found on mucins, glycoproteins, and gangliosides, MHC I glycoproteins, carbohydrate components found on membrane glycoproteins, including, mannose, N-acetyl-galactosamine, N-acetyl-glucosamine, fucose, galactose, and the like.

In particular embodiments, a heparan sulfate (HS) or heparin binding domain is substituted into the modified AAV capsid protein (for example, in an AAV that otherwise does not bind to HS or heparin). It is known in the art that HS/heparin binding is mediated by a “basic patch” that is rich in arginines and/or lysines. In exemplary embodiments, a sequence following the motif BXXB, where “B” is a basic residue and X is neutral and/or hydrophobic. As one nonlimiting example, BXXB is RGNR. In particular embodiments, BXXB is substituted for amino acid positions 462 through 465 in the native AAV2 capsid protein or the corresponding position in the capsid protein of another AAV.

Other nonlimiting examples of suitable targeting sequences include the peptides targeting coronary artery endothelial cells identified by Müller et al., Nature Biotechnology 21:1040-1046 (2003) (e.g., consensus sequences NSVRDLG/S, PRSVTVP, NSVSSXS/A); tumor-targeting peptides as described by Grifman et al., Molecular Therapy 3:964-975 (2001) (e.g., NGR, NGRAHA); lung or brain targeting sequences as described by Work et al., Molecular Therapy 13:683-693 (2006) (e.g., QPEHSST, VNTANST, HGPMQKS, PHKPPLA, IKNNEMW, RNLDTPM, VDSHRQS, YDSKTKT, SQLPHQK, STMQQNT, TERYMTQ, QPEHSST, DASLSTS, DLPNKKT, DLTAARL, EPHQFNY, EPQSNHT, MSSWPSQ, NPKHNAT, PDGMRTT, PNNNKTT, QSTTHDS, TGSKQKQ, SLKHQAL and SPIDGEQ), vascular targeting sequences described by Hajitou et al., TCM 16:80-88 (2006) (WIFPWIQL, CDCRGDCFC, CNGRC, CPRECES, GSL, CTTHWGFTLC, CGRRAGGSC, CKGGRAKDC, and CVPELGHEC); targeting peptides as described by Koivunen et al., J. Nucl. Med. 40:883-888 (1999) (CRRETAWAK, KGD, VSWFSHRYSPFAVS, GYRDGYAGPILYN, XXXY*XXX [where Y* is phospho-Tyr], Y*E/MNW, RPLPPLP, APPLPPR, DVFYPYPYASGS, MYWYPY, DITWDQLWDLMK, CWDDG/LWLC, EWCEYLGGYLRCYA, YXCXXGPXTWXCXP, IEGPTLRQWLAARA, LWXXY/W/F/H, XFXXYLW, SSIISHFRWGLCD, MSRPACPPNDKYE, CLRSGRGC, CHWMFSPWC, WXXF, CSSRLDAC, CLPVASC, CGFECVRQCPERC, CVALCREACGEGC, SWCEPGWCR, YSGKWGW, GLSGGRS, LMLPRAD, CSCFRDVCC, CRDVVSVIC, CNGRC, and GSL); and tumor targeting peptides as described by Newton & Deutscher, Phage Peptide Display in Handbook of Experimental Pharmacology, pages 145-163, Springer-Verlag, Berlin (2008) (MARSGL, MARAKE, MSRTMS, KCCYSL, WRR, WKR, WVR, WVK, WIK, WTR, WVL, WLL, WRT, WRG, WVS, WVA, MYWGDSHWLQYWYE, MQLPLAT, EWLS, SNEW, TNYL, WIFPWIQL, WDLAWMFRLPVG, CTVALPGGYVRVC, CVPELGHEC, CGRRAGGSC, CVAYCIEHHCWTC, CVFAHNYDYLVC, and CVFTSNYAFC, VHSPNKK, CDCRGDCFC, CRGDGWC, XRGCDX, PXXS/T, CTTHWGFTLC, SGKGPRQITAL, A9A/Q)(N/A)(L/Y)(T/V/M/R)(R/K), VYMSPF, MQLPLAT, ATWLPPR, HTMYYHHYQHHL, SEVGCRAGPLQWLCEKYFG, CGLLPVGRPDRNVWRWLC, CKGQCDRFKGLPWEC, SGRSA, WGFP, LWXXAr [Ar=Y, W, F, H), XFXXYLW, AEPMPHSLNFSQYLWYT, WAY(W/F)SP, IELLQAR, DITWDQLWDLMK, AYTKCSRQWRTCMTTH, PQNSKIPGPTFLDPH, SMEPALPDWWWKMFK, ANTPCGPYTHDCPVKR, TACHQHVRMVRP, VPWMEPAYQRFL, DPRATPGS, FRPNRAQDYNTN, CTKNSYLMC, C(R/Q)L/RT(G/N)XXG(A/V)GC, CPIEDRPMC, HEWSYLAPYPWF, MCPKHPLGC, RMWPSSTVNLSAGRR, SAKTAVSQRVWLPSHRGGEP, KSREHVNNSACPSKRITAAL, EGFR, RVS, AGS, AGLGVR, GGR, GGL, GSV, GVS, GTRQGHTMRLGVSDG, IAGLATPGWSHWLAL, SMSIARL, HTFEPGV, NTSLKRISNKRIRRK, LRIKRKRRKRKKTRK, GGG, GFS, LWS, EGG, LLV, LSP, LBS, AGG, GRR, GGH and GTV).

In other embodiments of the invention, the targeting sequence is SIGYPLP, VNTANST, QPEHSST or SGRGDS.

As yet a further alternative, the targeting sequence may be a peptide that can be used for chemical coupling (e.g., can comprise arginine and/or lysine residues that can be chemically coupled through their R groups) to another molecule that targets entry into a cell. Such methods can also be used to chemically link a molecule to facilitate purifying and/or detecting the AAV capsid protein or virus capsid or virus vector comprising the same.

As discussed above with respect to HI loop mutants, one or more non-naturally occurring amino acids as described by Wang et al., Annu Rev Biophys Biomol Struct. 35:225-49 (2006)) can be incorporated into the AAV capsid protein at an orthogonal site as a means of redirecting a virus capsid or virus vector comprising the modified AAV capsid subunit to a desired target tissue(s). These unnatural amino acids can advantageously be used to chemically link molecules of interest to the AAV capsid protein including without limitation: glycans (mannose-dendritic cell targeting); RGD, bombesin or a neuropeptide for targeted delivery to specific cancer cell types; RNA aptamers or peptides selected from phage display targeted to specific cell surface receptors such as growth factor receptors, integrins, and the like. Methods of chemically modifying amino acids are known in the art (see, e.g., Greg T. Hermanson, Bioconjugate Techniques, 1^{st} edition, Academic Press, 1996).

Further, in embodiments of the invention one or more amino acid residues from the HI loop of another parvovirus (e.g., AAV) can be substituted into the HI loop of the AAV capsid protein. In particular embodiments, 2, 3, 4, 5, 6, 7, 8, 9, 10, 12 o4 14 amino acids from another parvovirus can be substituted into the HI loop of the AAV capsid protein. In nonlimiting embodiments, the AAV capsid to be modified is an AAV1, AAV2, AAV3, AAV4, AAV5, AAV6, AAV7, AAV8, AAV9, AAV10, AAV11 capsid protein or the capsid protein of any other AAV described herein or now known or later discovered and the amino acids to be substituted in are derived from the amino acid sequence of the HI loop of an AAV1, AAV2, AAV3, AAV4, AAV5, AAV6, AAV7, AAV8, AAV9, AAV10, AAV11 capsid protein or the capsid protein of any other AAV described herein or now known or later discovered (as long as the capsid protein to be modified and the sequence to be substituted therein are derived from different AAV). As further examples, in particular embodiments, 1, 2, 3, 4, 5, 6, 7, 8, 9, 10, 12 or 14 amino acids from the HI loop of AAV4 or AAV5 are incorporated into the HI loop of an AAV2 capsid protein (e.g., the amino acids at positions 661-666 of the AAV4 capsid protein are substituted at amino acid positions 662 to 667 of the AAV2 capsid protein).

The invention contemplates that the modified AAV capsid proteins of the invention can be produced by modifying the capsid protein of any AAV now known or later discovered. Further, the AAV capsid protein that is to be modified can be a native AAV capsid protein (e.g., an AAV2, AAV3a or 3b, AAV4, AAV5, AAV8, AAV9, AAV10 or AAV11 capsid protein or any of the AAV shown in Table 3) but is not so limited. Those skilled in the art will understand that a variety of manipulations to the AAV capsid proteins are known in the art and the invention is not limited to modifications of naturally occurring AAV capsid proteins. For example, the capsid protein to be modified may already have alterations as compared with naturally occurring AAV (i.e., is derived from a naturally occurring AAV capsid protein, e.g., AAV2, AAV3a, AAV3b, AAV4, AAV5, AAV6, AAV7, AAV8, AAV9, AAV10 and/or AAV11 or any other AAV now known or later discovered). Such AAV capsid proteins are also within the scope of the present invention.

For example, the AAV capsid protein can comprise an amino acid insertion directly following amino acid 264 of the native AAV2 capsid protein sequence (see, e.g., WO 2006/066066) and/or can comprise an inner loop mutation as described in the United States provisional application filed Feb. 11, 2009 by Asokan et al. entitled “Modified Virus Vectors and Methods of Making and Using the Same.” As another illustrative example, the AAV capsid protein to be modified according to the present invention can have a peptide targeting sequence incorporated therein.

Thus, in particular embodiments, the AAV capsid protein to be modified can be derived from a naturally occurring AAV but further comprise one or more foreign sequences (e.g., that are exogenous to the native virus) that are inserted and/or substituted into the capsid protein and/or has been altered by deletion of one or more amino acids.

Accordingly, when referring herein to a specific AAV capsid protein (e.g., an AAV2, AAV3, AAV4, AAV5, AAV6, AAV7, AAV8, AAV9, AAV10 or AAV11 capsid protein or a capsid protein from any of the AAV shown in Table 3, etc.), it is intended to encompass the native capsid protein as well as capsid proteins that have alterations other than the modifications of the invention. Such alterations include substitutions, insertions and/or deletions. In particular embodiments, the AAV capsid protein to be modified comprises 1, 2, 3, 4, 5, 6, 7, 8, 9, 10, 11, 12, 13, 14, 15, 16, 17, 18, 19 or 20, less than 20, less than 30, less than 40, less than 50, less than 60, or less than 70 amino acids inserted therein (other than the insertions of the present invention) as compared with the native AAV capsid protein sequence. In embodiments of the invention, the AAV capsid protein to be modified comprises 1, 2, 3, 4, 5, 6, 7, 8, 9, 10, 11, 12, 13, 14, 15, 16, 17, 18, 19 or 20, less than 20, less than 30, less than 40, less than 50, less than 60, or less than 70 amino acid substitutions (other than the amino acid substitutions according to the present invention) as compared with the native AAV capsid protein sequence. In embodiments of the invention, the AAV capsid protein to be modified comprises a deletion of 1, 2, 3, 4, 5, 6, 7, 8, 9, 10, 11, 12, 13, 14, 15, 16, 17, 18, 19 or 20, less than 20, less than 30, less than 40, less than 50, less than 60, or less than 70 amino acids (other than the amino acid deletions of the invention) as compared with the native AAV capsid protein sequence.

Thus, for example, the term “AAV2 capsid protein” includes AAV capsid proteins having the native AAV2 capsid protein sequence (see GenBank Accession No. AAC03780) as well as those comprising substitutions, insertions and/or deletions (as described in the preceding paragraph) in the native AAV2 capsid protein sequence.

In particular embodiments, the AAV capsid protein has the native AAV capsid protein sequence or has an amino acid sequence that is at least about 90%, 95%, 97%, 98% or 99% similar or identical to a native AAV capsid protein sequence. For example, in particular embodiments, an “AAV2 capsid protein” encompasses the native AAV2 capsid protein amino acid sequence as well as amino acid sequences that are at least about 90%, 95%, 97%, 98% or 99% similar or identical to the native AAV2 capsid protein sequence.

Methods of determining sequence similarity or identity between two or more amino acid sequences are known in the art. Sequence similarity or identity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), or by inspection.

Another suitable algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al., (1997) Nucleic Acids Res. 25, 3389-3402.

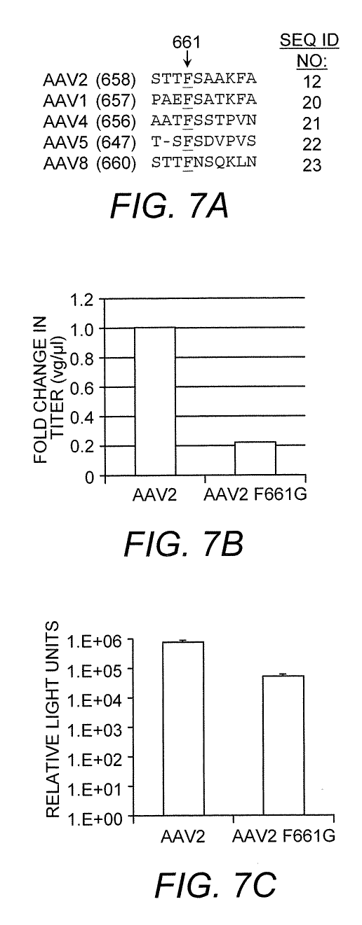

In representative embodiments of the invention, a modification is made in the region of amino acid positions 662 to 667 (inclusive) of the native AAV2 capsid protein (using VP1 numbering) or the corresponding positions of another AAV. The amino acid positions in other AAV that “correspond to” positions 662 to 667 (or any other positions in the HI loop) of the native AAV2 capsid protein will be apparent to those skilled in the art and can be readily determined using sequence alignment techniques (see, e.g., FIG. 7 of WO 2006/066066) and/or crystal structure analysis (Padron et al., (2005) J. Virol. 79:5047-58) (see also Table 5).

To illustrate, the modification can be introduced into an AAV capsid protein that already contains insertions and/or deletions such that the position of all downstream sequences is shifted. In this situation, the amino acid positions corresponding to amino acid positions 662 to 667 in the native AAV2 capsid protein would still be readily identifiable to those skilled in the art. To illustrate, the capsid protein can be an AAV2 capsid protein that contains an insertion following amino acid position 264 (see, e.g., WO 2006/066066). The amino acids found at positions 662 through 667 (e.g., SAAKFA in the native AAV2 capsid protein) would now be at positions 663 through 668 but would still be identifiable to those skilled in the art.

The invention also provides a virus capsid comprising, consisting essentially of, or consisting of a modified AAV capsid protein of the invention. In particular embodiments, the virus capsid is a parvovirus capsid, which may further be an autonomous parvovirus capsid or a dependovirus capsid. Optionally, the virus capsid is an AAV capsid. In particular embodiments, the AAV capsid is an AAV1, AAV2, AAV3a, AAV3b, AAV4, AAV5, AAV6, AAV7, AAV8, AAV9, AAV10, AAV11 or any other AAV shown in Table 3 or is derived from any of the foregoing by one or more insertions, substitutions and/or deletions.

In embodiments of the invention, the modified virus capsid comprises about 1, 2, 3, 4, 5, 10, 12, 15, 18, 20, 25, 30, 35, 40, 45, 50, 55 or 60 copies of the modified capsid protein of the invention (including VP1, VP2 and/or VP3). In further exemplary embodiments, the modified virus capsid comprises about 5-50, 5-45, 5-40, 5-35, 5-30, 5-25, 5-20, 5-15, 10-50, 10-45, 10-40, 10-35, 10-30, 10-25, 10-20, 12-60, 12-50, 12-45, 12-40, 12-35, 12-30, 12-25, 12-120, 15-60, 15-50, 15-45, 15-40, 15-35, 15-30, 15-25, 20-60, 20-50, 20-45, 20-40, 20-35, 20-30, 25-60, 25-50, 25-45, 25-40, 30-60, 30-50, 30-45, or 40-60 copies of the modified capsid protein of the invention (including VP1, VP2 and/or VP3).

In representative embodiments, the modified virus capsid is an AAV capsid and comprises about a 1:1, 1.5:1, 2:1, 2.5:1, 3:1, 3.5:1, 4:1, 4.5:1, 5:1, 6:1, 7:1, 8:1, 9:1, 10:1, 12:1, 15:1, 20:1 or 30:1 ratio of AAV capsid proteins that do not comprise the HI loop modifications of the invention to the AAV capsid proteins comprising the HI loop modifications of the invention. Exemplary ratios include without limitation: about a 1-30:1, about a 1-20:1, about a 1-15:1, about a 1-12:1, about a 1-10:1, about a 1-8:1, about a 1-6:1 about a 1-5:1, about a 1-4:1, about a 1-3:1, about a 2-30:1, about a 2-20:1, about a 2-15:1, about a 2-12:1, about a 2-10:1, about a 2-8:1, about a 2-6:1, about a 2-5:1, about a 2-4:1, about a 3-30:1, about a 3-20:1, about a 3-15:1, about a 3-12:1, about a 3-10:1, about a 3-8:1, about a 3-6:1, about a 3-5:1, about a 4-30:1, about a 4-20:1, about a 4-15:1, about a 4-12:1, about a 4-10:1, about a 4-8:1, about a 4-6:1, about a 5-30:1, about a 5-20:1, about a 4-15:1, about a 5-12:1, about a 5-10:1, about a 5-8:1, about a 6-30:1, about a 6-20:1, about a 6-15:1, about a 6-12:1, about a 6-10:1, about a 6-8:1, about a 8-30:1, about a 8-20:1, about a 8-15:1, about a 8-12:1, about a 8-10:1, about a 10-30:1, about a 10-20:1, about a 10-15:1, about a 10-12:1, about a 12-30:1, about a 12-20:1, about a 12-15:1, about a 15-30:1, about a 15-20:1, or about a 20-30:1 ratio of AAV capsid proteins that do not comprise the HI loop modifications of the invention to the AAV capsid proteins comprising the HI loop modifications of the invention.

The modified virus capsids can be used as “capsid vehicles,” as has been described, for example, in U.S. Pat. No. 5,863,541. Molecules that can be packaged by the modified virus capsid and transferred into a cell include heterologous DNA, RNA, polypeptides, small organic molecules, or combinations of the same.

Heterologous molecules are defined as those that are not naturally found in an AAV infection, e.g., those not encoded by a wild-type AAV genome. Further, therapeutically useful molecules can be associated with the outside of the chimeric virus capsid for transfer of the molecules into host target cells. Such associated molecules can include DNA, RNA, small organic molecules, carbohydrates, lipids and/or polypeptides. In one embodiment of the invention the therapeutically useful molecule is covalently linked (i.e., conjugated or chemically coupled) to the capsid proteins. Methods of covalently linking molecules are known by those skilled in the art.

The modified virus capsids of the invention also find use in raising antibodies against the novel capsid structures.

In other embodiments, the virus capsids can be administered to block certain cellular sites prior to and/or concurrently with (e.g., within minutes or hours of each other) administration of a virus vector delivering a nucleic acid encoding a polypeptide or functional RNA of interest. For example, the inventive capsids can be delivered to block cellular receptors on liver cells and a delivery vector can be administered subsequently or concurrently, which may reduce transduction of liver cells, and enhance transduction of other targets (e.g., skeletal muscle).

According to representative embodiments, modified virus capsids can be administered to a subject prior to and/or concurrently with a modified virus vector according to the present invention. Further, the invention provides compositions and pharmaceutical formulations comprising the inventive modified virus capsids; optionally, the composition also comprises a modified virus vector of the invention.

The invention also provides nucleic acids (optionally, isolated nucleic acids) encoding the modified AAV virus capsids and capsid proteins of the invention. Further provided are vectors comprising the nucleic acids, and cells (in vivo or in culture) comprising the nucleic acids and/or vectors of the invention. Suitable vectors include without limitation viral vectors (e.g., adenovirus, AAV, herpesvirus, vaccinia, poxviruses, baculoviruses, and the like), plasmids, phage, YACs, BACs, and the like. Such nucleic acids, vectors and cells can be used, for example, as reagents (e.g., helper packaging constructs or packaging cells) for the production of modified virus capsids or virus vectors as described herein.

Virus capsids according to the invention can be produced using any method known in the art, e.g., by expression from a baculovirus (Brown et al., (1994) Virology 198:477-488).

The invention also encompasses virus vectors comprising the modified capsid proteins and virus capsids of the invention. In particular embodiments, the virus vector is a parvovirus vector (e.g., comprising a parvovirus capsid and/or vector genome), for example, an AAV vector (e.g., comprising an AAV capsid and/or vector genome). In representative embodiments, the virus vector comprises a modified AAV capsid comprising a modified AAV capsid protein of the invention and a vector genome.

For example, in representative embodiments, the virus vector comprises: (a) a modified virus capsid (e.g., a modified AAV capsid) comprising a modified AAV capsid protein of the invention; and (b) a nucleic acid comprising a terminal repeat sequence (e.g., an AAV TR), wherein the nucleic acid comprising the terminal repeat sequence is encapsidated by the modified virus capsid. The nucleic acid can optionally comprise two terminal repeats (e.g., two AAV TRs).

In representative embodiments, the virus vector is a recombinant virus vector comprising a heterologous nucleic acid encoding a polypeptide or functional RNA of interest. Recombinant virus vectors are described in more detail below.

### Purification Methods.

As discussed herein, the present invention provides methods of incorporating sequences that facilitate purification into the HI loop (e.g., at amino acid positions 662 to 667 of the native AAV2 capsid subunit or the corresponding positions of another AAV), for example, by affinity purification techniques (including immunopurification techniques). Thus, for example, if a poly-histidine sequence is incorporated into the HI loop, affinity chromatography using a matrix that comprises nickel can be used to purify the modified AAV capsid protein or a virus capsid or virus vector comprising the same by employing methods that are well-known in the art. In the case of an antigenic peptide (such as FLAG), immunopurification methods can be used to purify the modified AAV capsid protein comprising the antigenic peptide incorporated into the HI loop or a virus capsid or virus vector comprising the same using routine methods known to those skilled in the art.

Thus, in particular embodiments, the invention provides a method of purifying an AAV capsid protein or a virus capsid or virus vector comprising the same from a sample, the method comprising: (a) providing a solid support comprising a matrix, wherein the matrix comprises nickel (e.g., nickel ion); (b) contacting the solid support with a sample comprising the AAV capsid protein, virus capsid and/or virus vector comprising a poly-histidine tag according to the present invention; and (c) eluting the bound AAV capsid protein, virus capsid and/or virus vector from the matrix. In particular embodiments, the virus capsid or virus vector is an AAV capsid or AAV vector, respectively.

Matrices comprising nickel for affinity purification of proteins comprising poly-histidine tags are known in the art. Binding of the capsid proteins, virus capsids and capsid proteins to the Ni-matrix can be carried out by any method known in the art as can elution from the matrix. In particular embodiments, elution is achieved by increasing the concentration of imidazole and/or salt.

In other representative embodiments, the invention provides a method of purifying an AAV capsid protein or a virus capsid or virus vector comprising the same from a sample, the method comprising: (a) providing a solid support comprising a matrix, wherein the matrix comprises an antibody; (b) contacting the solid support with a sample comprising an AAV capsid protein, virus capsid and/or virus vector according to the present invention comprising a peptide sequence that is recognized by the antibody; and (c) eluting the bound AAV capsid protein, virus capsid and/or virus vector from the matrix. In particular embodiments, the virus capsid or virus vector is an AAV capsid and AAV vector, respectively. Elution can be achieved, for example, by increasing the salt concentration and/or by adding excess free antibody or ligand.

In still further embodiments, the invention provides a method of purifying an AAV capsid protein or a virus capsid or virus vector comprising the same from a sample, the method comprising: (a) providing a solid support comprising a matrix, wherein the matrix comprises streptavidin; (b) contacting the solid support with a sample comprising an AAV capsid protein, virus capsid and/or virus vector according to the present invention comprising a streptavidin affinity peptide; and (c) eluting the bound AAV capsid protein, virus capsid and/or virus vector from the matrix. In particular embodiments, the virus capsid or virus vector is an AAV capsid and AAV vector, respectively. Elution can be achieved, for example, by increasing the salt concentration or adding biotin or another streptavidin ligand. Affinity matrices (including magnetic beads) comprising streptavidin are routine in the art and are commercially available.

The sample can be any sample that contains, or is suspected of containing, an AAV capsid protein according to the present invention or a virus capsid or virus vector comprising the same. The sample may be a crude sample (e.g., a lysed cell preparation), a partially-purified sample (e.g., the sample may be the result of ammonium sulfate precipitation, dialysis, density gradient purification, or any other purification method) or may be a relatively pure preparation (i.e., the method is practiced primarily for the purpose of concentrating or reducing the sample volume of the virus).

The solid support can be contacted with the sample containing (or suspected of containing) the modified AAV capsid protein or virus capsid and/or virus vector by any method known in the art. For example, the solid support can be packed into a chromatography column. Chromatography can be carried out using conventional columns or by HPLC (high performance liquid chromatography) or FPLC (fast protein liquid chromatography). Alternatively, the sample can be contacted in solution with the solid support (e.g., in the form of beads, such as magnetic beads) and purified by a batch method.

All known methods for immobilization of molecules (e.g., by adsorption, by electrostatic interactions, by covalent bonds) and any suitable matrix available to those skilled in the art may be employed in carrying out the present invention (see, e.g., Methods in Molecular Biology, Protein Purification Protocols (Shawn Doonan ed., 1996)). Matrices for use according to the present invention encompass solid and semi-solid matrices. Exemplary matrices include beads formed from glass, silica, alumina, ground corn grits, cellulose, agarose, or CELITE™ (a commercially available form of diatomaceous earth). In particular embodiments, the beads are magnetized. Typically, the matrix is modified to bear reactive groups to facilitate the immobilization reaction. For example, primary amine groups can be attached to the matrix by using silanes for siliceous or alumina-based supports. The attached primary amine groups are activated by glutaraldehyde or other activating agent prior to the addition of the ligand. Crosslinking of the covalently bound affinity ligand is optional.

### Methods of Producing Virus Vectors.

The present invention further provides methods of producing the inventive virus vectors. In one particular embodiment, the present invention provides a method of producing a virus vector, the method comprising providing to a cell: (a) a nucleic acid template comprising at least one TR sequence (e.g., AAV TR sequence), and (b) AAV sequences sufficient for replication of the nucleic acid template and encapsidation into AAV capsids (e.g., AAV rep sequences and AAV cap sequences encoding the AAV capsids of the invention). Optionally, the nucleic acid template further comprises at least one heterologous nucleic acid sequence. In particular embodiments, the nucleic acid template comprises two AAV ITR sequences, which are located 5′ and 3′ to the heterologous nucleic acid sequence (if present), although they need not be directly contiguous thereto.

The nucleic acid template and AAV rep and cap sequences are provided under conditions such that virus vector comprising the nucleic acid template packaged within the AAV capsid is produced in the cell. The method can further comprise the step of collecting the virus vector from the cell. The virus vector can be collected from the medium and/or by lysing the cells.

The cell can be a cell that is permissive for AAV viral replication. Any suitable cell known in the art may be employed. In particular embodiments, the cell is a mammalian cell. As another option, the cell can be a trans-complementing packaging cell line that provide functions deleted from a replication-defective helper virus, e.g., 293 cells or other E1a trans-complementing cells.

The AAV replication and capsid sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the E1a or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr, Top. Microbiol. Immun. 158:67).

As a further alternative, the rep/cap sequences may be stably incorporated into a cell.

Typically the AAV rep/cap sequences will not be flanked by the TRs, to prevent rescue and/or packaging of these sequences.

The nucleic acid template can be provided to the cell using any method known in the art. For example, the template can be supplied by a non-viral (e.g., plasmid) or viral vector. In particular embodiments, the nucleic acid template is supplied by a herpesvirus or adenovirus vector (e.g., inserted into the E1a or E3 regions of a deleted adenovirus). As another illustration, Palombo et al., (1998) J. Virology 72:5025, describes a baculovirus vector carrying a reporter gene flanked by the AAV TRs. EBV vectors may also be employed to deliver the template, as described above with respect to the rep/cap genes.

In another representative embodiment, the nucleic acid template is provided by a replicating rAAV virus. In still other embodiments, an AAV provirus comprising the nucleic acid template is stably integrated into the chromosome of the cell.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production as described by Ferrari et al., (1997) Nature Med. 3:1295, and U.S. Pat. Nos. 6,040,183 and 6,093,570.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by TRs.

Those skilled in the art will appreciate that it may be advantageous to provide the AAV replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes.

In one particular embodiment, the AAV rep/cap sequences and the adenovirus helper sequences are supplied by a single adenovirus helper vector. This vector further can further comprise the nucleic acid template. The AAV rep/cap sequences and/or the rAAV template can be inserted into a deleted region (e.g., the E1a or E3 regions) of the adenovirus.

In a further embodiment, the AAV rep/cap sequences and the adenovirus helper sequences are supplied by a single adenovirus helper vector. According to this embodiment, the rAAV template can be provided as a plasmid template.

In another illustrative embodiment, the AAV rep/cap sequences and adenovirus helper sequences are provided by a single adenovirus helper vector, and the rAAV template is integrated into the cell as a provirus. Alternatively, the rAAV template is provided by an EBV vector that is maintained within the cell as an extrachromosomal element (e.g., as an EBV based nuclear episome).

In a further exemplary embodiment, the AAV rep/cap sequences and adenovirus helper sequences are provided by a single adenovirus helper. The rAAV template can be provided as a separate replicating viral vector. For example, the rAAV template can be provided by a rAAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep/cap sequences and, if present, the rAAV template are embedded in the adenovirus backbone and are flanked by the 5′ and 3′ cis sequences, so that these sequences may be packaged into adenovirus capsids. As described above, the adenovirus helper sequences and the AAV rep/cap sequences are generally not flanked by TRs so that these sequences are not packaged into the AAV virions.

Zhang et al., ((2001) Gene Ther. 18:704-12) describe a chimeric helper comprising both adenovirus and the AAV rep and cap genes.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al., (1999) Gene Therapy 6:986 and WO 00/17377.

As a further alternative, the virus vectors of the invention can be produced in insect cells using baculovirus vectors to deliver the rep/cap genes and rAAV template as described, for example, by Urabe et al., (2002) Human Gene Therapy 13:1935-43.

AAV vector stocks free of contaminating helper virus may be obtained by any method known in the art. For example, AAV and helper virus may be readily differentiated based on size. AAV may also be separated away from helper virus based on affinity for a heparin substrate (Zolotukhin et al. (1999) Gene Therapy 6:973). Deleted replication-defective helper viruses can be used so that any contaminating helper virus is not replication competent. As a further alternative, an adenovirus helper lacking late gene expression may be employed, as only adenovirus early gene expression is required to mediate packaging of AAV virus. Adenovirus mutants defective for late gene expression are known in the art (e.g., ts100K and ts149 adenovirus mutants).

### Recombinant Virus Vectors.

The virus vectors of the present invention are useful for the delivery of nucleic acids to cells in vitro, ex vivo, and in vivo. In particular, the virus vectors can be advantageously employed to deliver or transfer nucleic acids to animal, including mammalian, cells.

Any heterologous nucleic acid sequence(s) of interest may be delivered in the virus vectors of the present invention. Nucleic acids of interest include nucleic acids encoding polypeptides, including therapeutic (e.g., for medical or veterinary uses) or immunogenic (e.g., for vaccines) polypeptides.

Therapeutic polypeptides include, but are not limited to, cystic fibrosis transmembrane regulator protein (CFTR), dystrophin (including mini- and micro-dystrophins, see, e.g, Vincent et al. (1993) Nature Genetics 5:130; U.S. Patent Publication No. 2003/017131; International publication WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sarcospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, ornithine transcarbamylase, β-globin, α-globin, spectrin, α₁-antitrypsin, adenosine deaminase, hypoxanthine guanine phosphoribosyl transferase, β-glucocerebrosidase, sphingomyelinase, lysosomal hexosaminidase A, branched-chain keto acid dehydrogenase, RP65 protein, cytokines (e.g., α-interferon, β-interferon, interferon-γ, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor-3 and -4, brain-derived neurotrophic factor, bone morphogenic proteins [including RANKL and VEGF], glial derived growth factor, transforming growth factor-α and -β, and the like), lysosomal acid α-glucosidase, α-galactosidase A, receptors (e.g., the tumor necrosis growth factors soluble receptor), S100A1, parvalbumin, adenylyl cyclase type 6, a molecule that effects G-protein coupled receptor kinase type 2 knockdown such as a truncated constitutively active bARKct, anti-inflammatory factors such as IRAP, anti-myostatin proteins, aspartoacylase, monoclonal antibodies (including single chain monoclonal antibodies; an exemplary Mab is the Herceptin® Mab). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. AAV vectors can also be used to deliver monoclonal antibodies and antibody fragments, for example, an antibody or antibody fragment directed against myostatin (see, e.g., Fang et al., Nature Biotechnology 23:584-590 (2005)).

Heterologous nucleic acid sequences encoding polypeptides include those encoding reporter polypeptides (e.g., an enzyme). Reporter polypeptides are known in the art and include, but are not limited to, Green Fluorescent Protein, 3-galactosidase, alkaline phosphatase, luciferase, and chloramphenicol acetyltransferase gene.

Alternatively, in particular embodiments of this invention, the heterologous nucleic acid may encode an antisense nucleic acid, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. No. 6,013,487; U.S. Pat. No. 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10:132-142 (2008) and Li et al., Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), and RNAi directed against pathogenic organisms and viruses (e.g., hepatitis B virus, human immunodeficiency virus, CMV, herpes simplex virus, human papilloma virus, etc.).

The virus vector may also comprise a heterologous nucleic acid that shares homology with and recombines with a locus on a host chromosome. This approach can be utilized, for example, to correct a genetic defect in the host cell.

The present invention also provides virus vectors that express an immunogenic polypeptide, e.g., for vaccination. The nucleic acid may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The use of parvoviruses as vaccine vectors is known in the art (see, e.g., Miyamura et al., (1994) Proc. Nat. Acad. Sci USA 91:8507; U.S. Pat. No. 5,916,563 to Young et al., U.S. Pat. No. 5,905,040 to Mazzara et al., U.S. Pat. No. 5,882,652, U.S. Pat. No. 5,863,541 to Samulski et al.). The antigen may be presented in the parvovirus capsid. Alternatively, the antigen may be expressed from a heterologous nucleic acid introduced into a recombinant vector genome. Any immunogen of interest as described herein and/or as is known in the art can be provided by the virus vector of the present invention.

An immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pot and env genes products). The immunogenic polypeptide can also be an arenavirus immunogen (e.g., Lassa fever virus immunogen, such as the Lassa fever virus nucleocapsid protein and the Lassa fever envelope glycoprotein), a poxvirus immunogen (e.g., a vaccinia virus immunogen, such as the vaccinia L1 or L8 gene products), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpes immunogen (e.g., CMV, EBV, HSV immunogens) a mumps immunogen, a measles immunogen, a rubella immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell. Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) Cancer Res. 54:3124), MART-1, gp100 MAGE-1, MAGE-2, MAGE-3, CEA, TRP-1, TRP-2, P-15, tyrosinase (Brichard et al., (1993) J. Exp. Med. 178:489); HER-2/neu gene product (U.S. Pat. No. 4,968,603), CA 125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

As a further alternative, the heterologous nucleic acid can encode any polypeptide that is desirably produced in a cell in vitro, ex vivo, or in vivo. For example, the virus vectors may be introduced into cultured cells and the expressed gene product isolated therefrom.

It will be understood by those skilled in the art that the heterologous nucleic acid(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the heterologous nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the heterologous nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the heterologous nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

The virus vectors according to the present invention provide a means for delivering heterologous nucleic acids into a broad range of cells, including dividing and non-dividing cells. The virus vectors can be employed to deliver a nucleic acid of interest to a cell in vitro, e.g., to produce a polypeptide in vitro or for ex vivo gene therapy. The virus vectors are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The virus vectors can also be used to produce a polypeptide of interest or functional RNA in cultured cells or in a subject (e.g., using the subject as a bioreactor to produce the polypeptide or to observe the effects of the functional RNA on the subject, for example, in connection with screening methods).

In general, the virus vectors of the present invention can be employed to deliver a heterologous nucleic acid encoding a polypeptide or functional RNA to treat and/or prevent any disease state for which it is beneficial to deliver a therapeutic polypeptide or functional RNA. Illustrative disease states include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders, Alzheimer's disease (GDF; neprilysin), multiple sclerosis (β-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product), diabetes mellitus (insulin), muscular dystrophies including Duchenne (dystrophin, mini-dystrophin, insulin-like growth factor 1, a sarcoglycan [e.g., α, β, γ], RNAi against myostatin, myostatin propeptide, follistatin, activin type II soluble receptor, anti-inflammatory polypeptides such as the Ikappa B dominant mutant, sarcospan, utrophin, mini-utrophin, RNAi against splice junctions in the dystrophin gene to induce exon skipping [see, e.g., WO/2003/095647], antisense against U7 snRNAs to induce exon skipping [see, e.g., WO/2006/021724], and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic defects, congenital emphysema (α1-antitrypsin), Lesch-Nyhan Syndrome (hypoxanthine guanine phosphoribosyl transferase), Niemann-Pick disease (sphingomyelinase), Tays Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1), serca2a, zinc finger proteins that regulate the phospholamban gene, Barkct, β2-adrenergic receptor, β2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor 1), kidney deficiency (erythropoietin), anemia (erythropoietin), arthritis (anti-inflammatory factors such as IRAP and TNFα soluble receptor), hepatitis (α-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

Gene transfer has substantial potential use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, virus vectors according to the present invention permit the treatment and/or prevention of genetic diseases.

The virus vectors according to the present invention may also be employed to provide a functional RNA to a cell in vitro or in vivo. Expression of the functional RNA in the cell, for example, can diminish expression of a particular target protein by the cell. Accordingly, functional RNA can be administered to decrease expression of a particular protein in a subject in need thereof. Functional RNA can also be administered to cells in vitro to regulate gene expression and/or cell physiology, e.g., to optimize cell or tissue culture systems or in screening methods.

Virus vectors according to the instant invention find use in diagnostic and screening methods, whereby a nucleic acid of interest is transiently or stably expressed in a cell culture system, or alternatively, a transgenic animal model.

The virus vectors of the present invention can also be used for various non-therapeutic purposes, including but not limited to use in protocols to assess gene targeting, clearance, transcription, translation, etc., as would be apparent to one skilled in the art. The virus vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

As a further aspect, the virus vectors of the present invention may be used to produce an immune response in a subject. According to this embodiment, a virus vector comprising a heterologous nucleic acid sequence encoding an immunogenic polypeptide can be administered to a subject, and an active immune response is mounted by the subject against the immunogenic polypeptide. Immunogenic polypeptides are as described hereinabove. In some embodiments, a protective immune response is elicited.

Alternatively, the virus vector may be administered to a cell ex vivo and the altered cell is administered to the subject. The virus vector comprising the heterologous nucleic acid is introduced into the cell, and the cell is administered to the subject, where the heterologous nucleic acid encoding the immunogen can be expressed and induce an immune response in the subject against the immunogen. In particular embodiments, the cell is an antigen-presenting cell (e.g., a dendritic cell).

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof.

In particular embodiments, the virus vector or cell comprising the heterologous nucleic acid can be administered in an immunogenically effective amount, as described below.

The virus vectors of the present invention can also be administered for cancer immunotherapy by administration of a virus vector expressing one or more cancer cell antigens (or an immunologically similar molecule) or any other immunogen that produces an immune response against a cancer cell. To illustrate, an immune response can be produced against a cancer cell antigen in a subject by administering a virus vector comprising a heterologous nucleic acid encoding the cancer cell antigen, for example to treat a patient with cancer and/or to prevent cancer from developing in the subject. The virus vector may be administered to a subject in vivo or by using ex vivo methods, as described herein. Alternatively, the cancer antigen can be expressed as part of the virus capsid or be otherwise associated with the virus capsid as described above.

As another alternative, any other therapeutic nucleic acid (e.g., RNAi) or polypeptide (e.g., cytokine) known in the art can be administered to treat and/or prevent cancer.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified. In representative embodiments, the invention provides a method of treating and/or preventing tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In particular embodiments, these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

In particular embodiments, cells may be removed from a subject with cancer and contacted with a virus vector according to the instant invention. The modified cell is then administered to the subject, whereby an immune response against the cancer cell antigen is elicited. This method can be advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., α-interferon, β-interferon, γ-interferon, ω-interferon, τ-interferon, interleukin-1α, interleukin-1 β, interleukin-2, interleukin-3, interleukin-4, interleukin 5, interleukin-6, interleukin-7, interleukin-8, interleukin-9, interleukin-10, interleukin-11, interleukin 12, interleukin-13, interleukin-14, interleukin-18, B cell Growth factor, CD40 Ligand, tumor necrosis factor-α, tumor necrosis factors, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the virus vector.

Cytokines may be administered by any method known in the art. Exogenous cytokines may be administered to the subject, or alternatively, a nucleic acid encoding a cytokine may be delivered to the subject using a suitable vector, and the cytokine produced in vivo.

### Subjects, Pharmaceutical Formulations, and Modes of Administration.

Virus vectors and capsids according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc. Human subjects include neonates, infants, juveniles and adults.

In particular embodiments, the present invention provides a pharmaceutical composition comprising a virus vector and/or capsid of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

One aspect of the present invention is a method of transferring a nucleic acid to a cell in vitro. The virus vector may be introduced into the cells at the appropriate multiplicity of infection according to standard transduction methods suitable for the particular target cells. Titers of virus vector to administer can vary, depending upon the target cell type and number, and the particular virus vector, and can be determined by those of skill in the art without undue experimentation. In representative embodiments, at least about 10³ infectious units, more preferably at least about 10⁵ infectious units are introduced to the cell.

The cell(s) into which the virus vector is introduced can be of any type, including but not limited to neural cells (including cells of the peripheral and central nervous systems, in particular, brain cells such as neurons and oligodendricytes), lung cells, cells of the eye (including retinal cells, retinal pigment epithelium, and corneal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin, as indicated above.

The virus vector can be introduced into cells in vitro for the purpose of administering the modified cell to a subject. In particular embodiments, the cells have been removed from a subject, the virus vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the recombinant virus vector can be introduced into cells from a donor subject, into cultured cells, or into cells from any other suitable source, and the cells are administered to a subject in need thereof (i.e., a “recipient” subject).

Suitable cells for ex vivo gene delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the virus vector are administered to the subject in a treatment effective or prevention effective amount in combination with a pharmaceutical carrier.

In some embodiments, the virus vector is introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In particular embodiments, the dosage is sufficient to produce a protective immune response (as defined above). The degree of protection conferred need not be complete or permanent, as long as the benefits of administering the immunogenic polypeptide outweigh any disadvantages thereof.

A further aspect of the invention is a method of administering the virus vector and/or virus capsid to subjects. Administration of the virus vectors and/or capsids according to the present invention to a human subject or an animal in need thereof can be by any means known in the art. Optionally, the virus vector and/or capsid is delivered in a treatment effective or prevention effective dose in a pharmaceutically acceptable carrier.

The virus vectors and/or capsids of the invention can further be administered to elicit an immunogenic response (e.g., as a vaccine). Typically, immunogenic compositions of the present invention comprise an immunogenically effective amount of virus vector and/or capsid in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the virus vector and/or capsid to be administered to a subject depend upon the mode of administration, the disease or condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10³, 10¹⁴, 10¹⁵ transducing units, optionally about 10⁸-10¹³ transducing units.

In particular embodiments, more than one administration (e.g., two, three, four or more administrations) may be employed to achieve the desired level of gene expression over a period of various intervals, e.g., daily, weekly, monthly, yearly, etc.

Exemplary modes of administration include oral, rectal, transmucosal, topical, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). Administration can also be to a tumor (e.g., in or near a tumor or a lymph node). The most suitable route in any given case will depend on the nature and severity of the condition being treated and/or prevented and on the nature of the particular vector that is being used.

Administration to skeletal muscle according to the present invention includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi guinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator internus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoid, sternohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

The virus vector and/or capsid can be delivered to skeletal muscle by intravenous administration, intra-arterial administration, intraperitoneal administration, limb perfusion, (optionally, isolated limb perfusion of a leg and/or arm; see, e.g. Arruda et al., (2005)Blood 105: 3458-3464), and/or direct intramuscular injection. In particular embodiments, the virus vector and/or capsid is administered to a limb (arm and/or leg) of a subject (e.g., a subject with muscular dystrophy such as DMD) by limb perfusion, optionally isolated limb perfusion.

Administration to cardiac muscle includes administration to the left atrium, right atrium, left ventricle, right ventricle and/or septum. The virus vector and/or capsid can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration to diaphragm muscle can be by any suitable method including intravenous administration, intra-arterial administration, and/or intra-peritoneal administration.

Delivery to a target tissue can also be achieved by delivering a depot comprising the virus vector and/or capsid. In representative embodiments, a depot comprising the virus vector and/or capsid is implanted into skeletal, cardiac and/or diaphragm muscle tissue or the tissue can be contacted with a film or other matrix comprising the virus vector and/or capsid. Such implantable matrices or substrates are described in U.S. Pat. No. 7,201,898.

In particular embodiments, a virus vector and/or virus capsid according to the present invention is administered to skeletal muscle, diaphragm muscle and/or cardiac muscle (e.g., to treat and/or prevent muscular dystrophy, heart disease [for example, PAD or congestive heart failure]).

In representative embodiments, the invention is used to treat and/or prevent disorders of skeletal, cardiac and/or diaphragm muscle.

In a representative embodiment, the invention provides a method of treating and/or preventing muscular dystrophy in a subject in need thereof, the method comprising: administering a treatment or prevention effective amount of a virus vector of the invention to a mammalian subject, wherein the virus vector comprises a heterologous nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-α2, α-sarcoglycan, β-sarcoglycan, γ-sarcoglycan, δ-sarcoglycan, IGF-1, an antibody or antibody fragment against myostatin or myostatin propeptide, and/or RNAi against myostatin. In particular embodiments, the virus vector can be administered to skeletal, diaphragm and/or cardiac muscle as described elsewhere herein.

Alternatively, the invention can be practiced to deliver a nucleic acid to skeletal, cardiac or diaphragm muscle, which is used as a platform for production of a polypeptide (e.g., an enzyme) or functional RNA (e.g., RNAi, microRNA, antisense RNA) that normally circulates in the blood or for systemic delivery to other tissues to treat and/or prevent a disorder (e.g., a metabolic disorder, such as diabetes (e.g., insulin), hemophilia (e.g., Factor IX or Factor VIII), a mucopolysaccharide disorder (e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.) or a lysosomal storage disorder (such as Gaucher's disease [glucocerebrosidase], Pompe disease [lysosomal acid α-glucosidase] or Fabry disease [α-galactosidase A]) or a glycogen storage disorder (such as Pompe disease [lysosomal acid a glucosidase]). Other suitable proteins for treating and/or preventing metabolic disorders are described above. The use of muscle as a platform to express a nucleic acid of interest is described in U.S. Patent publication US 2002/0192189.

Thus, as one aspect, the invention further encompasses a method of treating and/or preventing a metabolic disorder in a subject in need thereof, the method comprising: administering a treatment or prevention effective amount of a virus vector of the invention to skeletal muscle of a subject, wherein the virus vector comprises a heterologous nucleic acid encoding a polypeptide, wherein the metabolic disorder is a result of a deficiency and/or defect in the polypeptide. Illustrative metabolic disorders and heterologous nucleic acids encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art). Without being limited by any particular theory of the invention, according to this embodiment, administration to the skeletal muscle can result in secretion of the polypeptide into the systemic circulation and delivery to target tissue(s). Methods of delivering virus vectors to skeletal muscle is described in more detail herein.

The invention can also be practiced to produce antisense RNA, RNAi or other functional RNA (e.g., a ribozyme) for systemic delivery.

The invention also provides a method of treating and/or preventing congenital heart failure or PAD in a subject in need thereof, the method comprising administering a treatment or prevention effective amount of a virus vector of the invention to a mammalian subject, wherein the virus vector comprises a heterologous nucleic acid encoding, for example, a sarcoplasmic endoreticulum Ca²⁺-ATPase (SERCA2a), an angiogenic factor, phosphatase inhibitor I (I-1), RNAi against phospholamban; a phospholamban inhibitory or dominant-negative molecule such as phospholamban S16E, a zinc finger protein that regulates the phospholamban gene, β2-adrenergic receptor, β2-adrenergic receptor kinase (BARK), PI3 kinase, calsarcan, a β-adrenergic receptor kinase inhibitor (βARKct), inhibitor 1 of protein phosphatase 1, S100A1, parvalbumin, adenylyl cyclase type 6, a molecule that effects G-protein coupled receptor kinase type 2 knockdown such as a truncated constitutively active bARKct, Pim-1, PGC-1α, SOD-1, SOD-2, EC-SOD, kallikrein, HIF, thymosin-β4, mir-1, mir-133, mir-206 and/or mir-208.

Injectables can be prepared in conventional forms, either as liquid solutions or suspensions, solid forms suitable for solution or suspension in liquid prior to injection, or as emulsions. Alternatively, one may administer the virus vector and/or virus capsids of the invention in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1).

The virus vectors and/or virus capsids disclosed herein can be administered to the lungs of a subject by any suitable means, optionally by administering an aerosol suspension of respirable particles comprised of the virus vectors and/or virus capsids, which the subject inhales. The respirable particles can be liquid or solid. Aerosols of liquid particles comprising the virus vectors and/or virus capsids may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors and/or capsids may likewise be produced with any solid particulate medicament aerosol generator, by techniques known in the pharmaceutical art.

The virus vectors and virus capsids can be administered to tissues of the CNS (e.g., brain, eye) and may advantageously result in broader distribution of the virus vector or capsid than would be observed in the absence of the present invention.

In particular embodiments, the delivery vectors of the invention may be administered to treat diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulimia) and cancers and tumors (e.g., pituitary tumors) of the CNS.

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The delivery vectors of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a delivery vector of the invention.

Retinitis pigmentosa, by comparison, is characterized by retinal degeneration. In representative embodiments, retinitis pigmentosa can be treated by intraocular (e.g., vitreal administration) of a delivery vector encoding one or more neurotrophic factors.

Age-related macular degeneration involves both angiogenesis and retinal degeneration. This disorder can be treated by administering the inventive deliver vectors encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the inventive delivery vectors. Such agents include N-methyl-D-aspartate (NMDA) antagonists, cytokines, and neurotrophic factors, delivered intraocularly, optionally intravitreally.

In other embodiments, the present invention may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures over time.

In one representative embodiment, somatostatin (or an active fragment thereof) is administered to the brain using a delivery vector of the invention to treat a pituitary tumor. According to this embodiment, the delivery vector encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins are known in the art.

In particular embodiments, the vector can comprise a secretory signal as described in U.S. Pat. No. 7,071,172.

In representative embodiments of the invention, the virus vector and/or virus capsid is administered to the CNS (e.g., to the brain or to the eye). The virus vector and/or capsid may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The virus vector and/or capsid may also be administered to different regions of the eye such as the retina, cornea and/or optic nerve.

The virus vector and/or capsid may be delivered into the cerebrospinal fluid (e.g., by lumbar puncture) for more disperse administration of the delivery vector. The virus vector and/or capsid may further be administered intravascularly to the CNS in situations in which the blood-brain barrier has been perturbed (e.g., brain tumor or cerebral infarct).

The virus vector and/or capsid can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In particular embodiments, the virus vector and/or capsid is administered in a liquid formulation by direct injection (e.g., stereotactic injection) to the desired region or compartment in the CNS. In other embodiments, the virus vector and/or capsid may be provided by topical application to the desired region or by intra-nasal administration of an aerosol formulation. Administration to the eye, may be by topical application of liquid droplets. As a further alternative, the virus vector and/or capsid may be administered as a solid, slow-release formulation (see, e.g., U.S. Pat. No. 7,201,898).

In yet additional embodiments, the virus vector can used for retrograde transport to treat and/or prevent diseases and disorders involving motor neurons (e.g., amyotrophic lateral sclerosis (ALS); spinal muscular atrophy (SMA), etc.). For example, the virus vector can be delivered to muscle tissue from which it can migrate into neurons.

Having described the present invention, the same will be explained in greater detail in the following examples, which are included herein for illustration purposes only, and which are not intended to be limiting to the invention.

### EXAMPLES

### Example 1

### Surface Loop Dynamics in Adeno-Associated Virus Capsid Assembly

### Summary

In this study, the inventors have carried out a thorough characterization of the HI loop through deletion and substitution mutagenesis as well as a battery of biochemical assays to assess the role of this surface feature in the AAV life cycle. The results help demonstrate the plasticity of the HI loop and implicate a potential role in viral genome packaging. Simultaneously, the inventors identified a residue within the HI loop that dictates proper incorporation of VP1 in the viral capsid.

The HI loop is a prominent domain on the AAV capsid surface that extends from each viral protein (VP) subunit overlapping the neighboring five-fold VP. Despite the highly conserved nature of the residues at the five-fold pore, the HI loops surrounding this region vary significantly in amino acid sequence between the AAV serotypes. In order to understand the role of this unique capsid domain, we ablated side chain interactions between the HI loop and the underlying EF loop in the neighboring VP subunit by generating a collection of AAV2 deletion, insertion and substitution mutants. A mutant lacking the HI loop was unable to assemble particles while a substitution mutant (ten glycine residues) assembled particles but was unable to package viral genomes. Substitution mutants carrying corresponding regions from AAV1, AAV4, AAV5 and AAV8 yielded; a) particles with titers and infectivity identical to AAV2 (AAV2 HI1 & HI8), b) particles with decreased virus titer (one log), but normal infectivity (HI4), and c) particles that synthesized VPs but were unable to assemble into intact capsids (HI5). AAV5 HI is shorter than all other HI loops by one amino acid. Replacing the missing residue (threonine) in AAV2 HI5 resulted in a moderate particle assembly rescue. In addition, we substituted the HI loop with peptides varying in length and amino acid sequence. This region tolerated seven-amino acid peptide substitutions, unless spanning a conserved phenylalanine at amino acid position 661. Mutation of this highly conserved phenylalanine to a glycine resulted in a modest decrease in virus titer, but a substantial decrease (one log order) in infectivity. Subsequently, confocal studies revealed that AAV2 F661 G did not efficiently complete a key step in the infectious pathway, nuclear entry, hinting at a possible perturbation of VP1 phospholipase activity. Molecular modeling studies with the F661G mutant suggest that disruption of interactions between F661 and an underlying P373 residue in the EF loop of the neighboring subunit might adversely affect incorporation of the VP1 subunit at the five-fold axis. Western blot analysis confirmed inefficient incorporation of VP1 as well as a proteolytically processed VP1 subunit that could account for the markedly reduced infectivity. In summary, our studies show that the HI loop, while flexible in amino acid sequence, is involved in AAV capsid assembly, proper VP1 subunit incorporation, and viral genome packaging all of which implicate a potential role for this unique surface domain in viral infectivity.

### Generation of Mutants

All constructs were generated in the pXR2 (Rabinowitz et al. 2002 J Virol 76:791-801) backbone using primers and restriction sites for PCR or oligo insertion, respectively. PCR was used to generate AAV2 poly-glycine and AAV2 HI−/− mutants. PCR was performed using the Expand Long Template PCR kit from Roche. All other mutants generated were the result of enzyme digests and oligo insert ligations. Restriction sites were placed downstream and upstream of the HI loop, Sbf1 and Afe1 (pXSA), respectively. The HI loops from AAV4 and AAV5 were amplified with these restriction sites on the 5′ and 3′ ends, digested and inserted into the digested pXSA backbone. pXR1 and pXR8 were digested with Sbf1 and Afe1, removing the HI loop, which was then ligated into pXSA. Restriction enzyme sites were generated at amino acid position 648 (Age 1) and 666 (Nhe1) surrounding the HI loop in order to insert oligos into this region. Oligos were ordered with corresponding restriction sites at the 5′ and 3′ ends, digested, and ligated into the digested backbone. All oligos were synthesized by Integrated DNA Technologies. Site directed mutagenesis was also used in order to generate point mutations within the pXR2 backbone within the HI loop using the Stratagene QuikChange Site-Directed Mutagenesis kit. Primers generated are listed in Table 1.

### Virus Production

Virus was produced using the triple transfection method developed in our lab as described in Xiao et al. (1998) J Virol 72:2224-2232. Cells were transfected with pXR2 containing the capsid mutations, pXX6-80 helper plasmid, and pTR-CMV-Luciferase containing the luciferase reporter transgene flanked by terminal repeats. Cells were harvested 60 hrs post transfection and purified using cesium chloride gradient density centrifugation for 5 hrs at 65,000 rpm or overnight at 55,000 rpm. Gradients were fractionated and virus dialyzed against 1×PBS supplemented with calcium and magnesium. Viral titers were determined in triplicate by treating 2 μl of the virus fractions with DNase, digesting the capsid with proteinase-K and loading the viral genomic DNA on to a Hybond-XL membrane (Amersham). The viral DNA was detected using a ³²P-labeled probe complementary to the luciferase transgene. Some viruses were generated multiple times, for example: AAV2 HI−/−>five times; AAV2 HI4, two times; AAV2 HI5, >five times; and AAV2 F661G two times. Each mutant virus preparation was made in conjunction with control AAV2 for a transfection control and titer comparison. Representative titers and phenotypes were documented in the results.

### Western Dot Blot, Heat Treatment, and Western Blot

Production of empty and full capsids was determined post transfection by loading 2 ul of the virus fractions onto a nitrocellulose membrane in a dot blot apparatus. Membranes were blocked in 10% milk in PBS for 30 mins at RT and incubated with A20 primary antibody (dilution 1:20) (Wobus et al. (2000) J Virol 74:9281-9293) in 2% milk for 1 hour at RT. Membranes were washed 5 times with 1×PBS and incubated with goat anti-mouse horseradish peroxidase-conjugated secondary antibody (Pierce dilution 1:5000) for 30 mins. The membranes were washed as described above, and capsid production was visualized using the SuperSignal West Femto Maximum Sensitivity Substrate chemiluminescence kit from Pierce. To examine VP1 exposure, capsids were heat treated at a range of temperatures (Results) and blotted onto a nitrocellulose membrane through a dot blot apparatus. The membrane was incubated as described above except A1 (1:20) and B1 (1:20) (Wobus et al. (2000) J Virol 74:9281-9293) primary antibodies were used to detect VP1 exposure and capsid viral protein dissociation upon heat treatment, respectively. For western blotting approximately 1E10 dialyzed vg containing particles were mixed with NuPAGE LDS sample buffer (Invitrogen), run on a NuPAGE gel (Invitrogen), transferred to a nitrocellulose membrane (Invitrogen) and blotted as described above. Other antibodies used to analyze the VP1 unique region during western blotting were anti-aa15-29 (1:1000) and anti-aa60-74 (1:1000) (Pacific Immunology: Grieger, Samulski unpublished). All films were exposed anywhere from 10 seconds to 1 minute.

### Viral Transduction Assay

Viral transduction was analyzed by quantifying the luciferase transgene expression in 293-cell lysate no more than 24 hrs post infection. 2E5 293 cells were transduced with 3000 vector genomes (vg)/cell and lysed using 1× Passive Lysis Buffer provided by Promega. Relative light units were analyzed post addition of the D-luciferin substrate (NanoLight) to the cell lysates using a Victor2 Luminometer (PerkinElmer).

### Electron Microscopy

10 μl of purified and dialyzed full and empty virus particles in 1×PBS with Ca⁺⁺ and Mg⁺⁺ were pipetted onto a glow-discharged copper grid. The grid was washed twice with water and then stained with 2% uranyl acetate. EM images were taken with a LEO EM 910 TEM at varying magnifications at the University of North Carolina Microscopy Labs.

### Heparin Binding Assay

1E10 vector genome containing particles of virus were incubated with pre-equilibrated heparin type III-S agarose beads (Sigma). The flowthrough was collected and the beads washed two times with 1×PBS. The washes were collected, and the beads were washed with increasing salt concentrations from 0.2M to 0.6M PBS. A load control, the flowthrough, washes and elutions were blotted onto a nitrocellulose membrane using a dot blot apparatus. The membrane was blocked, incubated with antibody as described earlier in the methods. In this case, A20 was used as the primary antibody in order to detect intact capsids, and determine the affinity of virus mutants to heparin beads.

### Confocal Microscopy

Coverslips were plated with 50,000 Helas/slip in a 24 well plate. Each well was infected with 30,000 vg/cell. 12 hrs post infection cells were fixed with 2% paraformaldehyde and washed with 1×PBS. Cells were permeabilized with 0.1% Triton X-100 at room temp for 5 mins and washed with 1×PBS and 1× Immunofluorescence wash buffer (IFWB: dH2O, 20 mM Tris pH 7.5, 137 mM NaCl, 3 mM KCl, 1.5 mM MgCl2, 5 mg/ml BSA, 0.05% Tween). Cells incubated in A20 primary (1:10) in IFWB for 1 hr at 37° C. Cells were washed with 1×PBS and incubated with 488 nm fluorophore-conjugated secondary antibody (1:1250) in IFWB for 1 hr at 37° C. (Abcam). Coverslips were mounted onto slides using Prolong antifade gold with DAPI mounting media. Slides were viewed on a Leica microscope in the Michael Hooker Microscopy Facility at the University of North Carolina-Chapel Hill.

### Molecular Modeling Studies

Homologous models of HI loop mutants were generated using VIPER (Reddy et al. 2001 J Virol 75:11943-11947) and/or Swiss-Model programs in order to visualize the effects of mutagenesis on the virus capsid. The available structure of AAV2 (PDB accession No. 1 LP3) was supplied as a template for the mutant models generated in the AAV2 background. Once the Swiss-model pdbs were generated, the program 0 (Jones et al. (1991) Acta Cryst. A47, 110-119) was used to generate symmetry related models of the monomer subunits using icosahedral matrix multiplication. Structure visualization of mutant models and the structures of other AAV serotypes whose HI loops were substituted into AAV2 (e.g., AAV4, PDB accession No. 2G8G (Govindasamy et al. (2006) J Virol 80:11556-11570); AAV8, PDB accession No. 2QA0, AAV1 and AAV5 (unpublished) were performed using winCOOT® software and images were rendered with MacPyMOL® software.

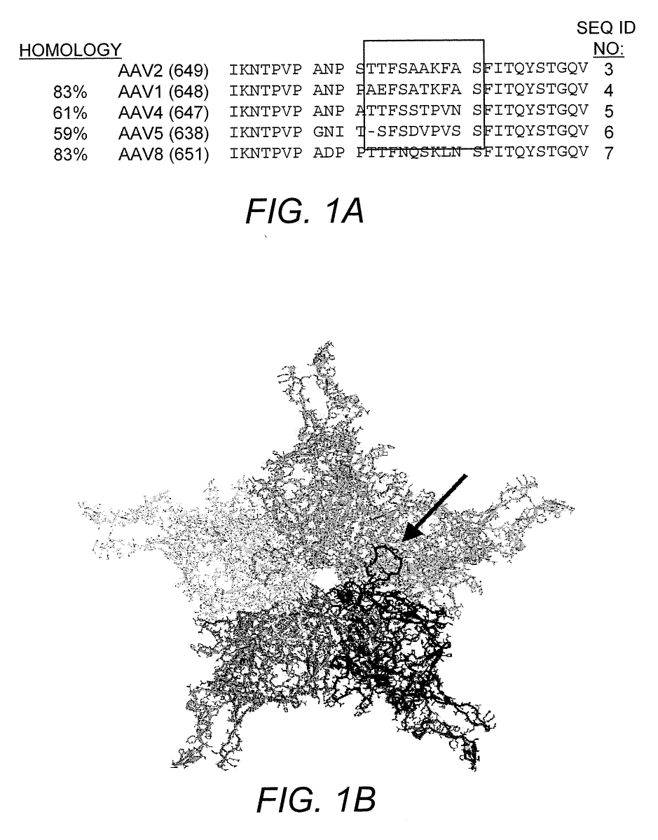

With respect to structure and topology, the HI loop is highly conserved between the AAV serotypes and autonomous parvoviruses (FIG. 1A); however the amino acid (aa) sequence varies significantly. In order to determine the role of the HI loop as it pertains to the AAV capsid structure and life cycle, the AAV2 HI loop sequence was mutated, swapped between serotypes or substituted, and the resulting viruses assayed for viral assembly, encapsidation of the viral DNA, binding to heparan sulfate, and the ability to successfully infect target cells. Mutagenesis was performed on the ten varying amino acids of the HI loop, starting with serine 658 and ending with alanine 667 in the AAV2 capsid (FIG. 1A).

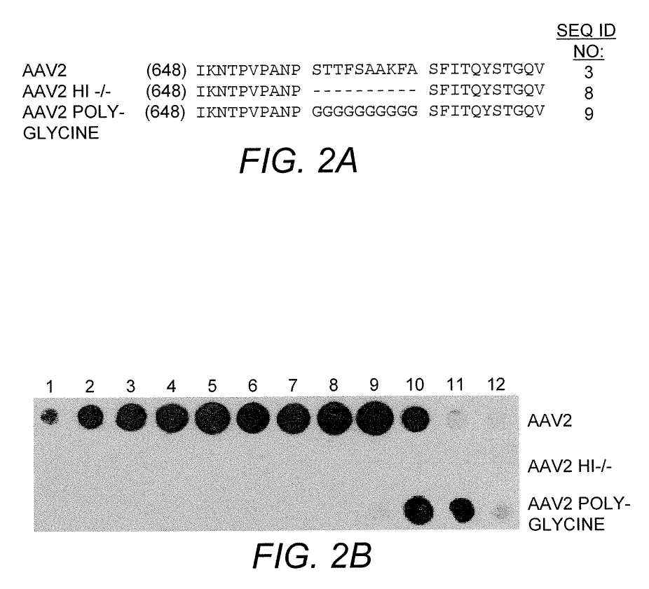

Deletion and glycine substitution mutants. First, mutations were generated in which the AAV2 HI loop amino acids were either deleted (AAV2 HI−/−) or substituted with a poly-glycine peptide (AAV2 poly-glycine). In the substitution mutant the ten most variable HI loop amino acids were replaced with a chain of glycine residues in order to generate a flexible loop structure devoid of all amino acid side chain contacts between this loop and the EF loop (located between the β-strands βE and βF) in the underlying subunit (FIG. 2A). Upon removal of the HI loop, AAV2 HI−/− capsid viral proteins Were expressed but unable to assemble into particles as determined by A20 western dot blot analysis (FIG. 2B) and DNA dot blot. As shown in FIG. 2B, the AAV2 poly-glycine mutant formed AAV particles based on western dot blot analysis (see fractions 10 and 11 of the cesium chloride gradient), however, these particles were devoid of DNA (Table 2). The AAV2 poly-glycine empty particles appear to be similar to AAV2 empty particles when analyzed under EM (data not shown), with the exception of a ring-like staining pattern more apparent with AAV2 poly-glycine particles. Although no other gross structural changes were evident by EM, potential conformational changes to the capsid were suggested by the ring-like staining therefore, further biochemical analyses were carried out such as heparin binding affinity chromatography. The AAV2 poly-glycine mutant had a comparable affinity for heparin sulfate as wt AAV2 particles as determined through a heparin binding assay, eluting from the heparin column mostly at 0.4M salt (Table 2). From these data, three important conclusions can be drawn; the HI loop structure is necessary for capsid assembly, and specific amino acid side-chain interactions within this loop appear to be necessary for packaging the viral genomic DNA. In addition, residues forming the HI loop and adjacent capsid regions do not play a role in heparan sulfate receptor attachment.

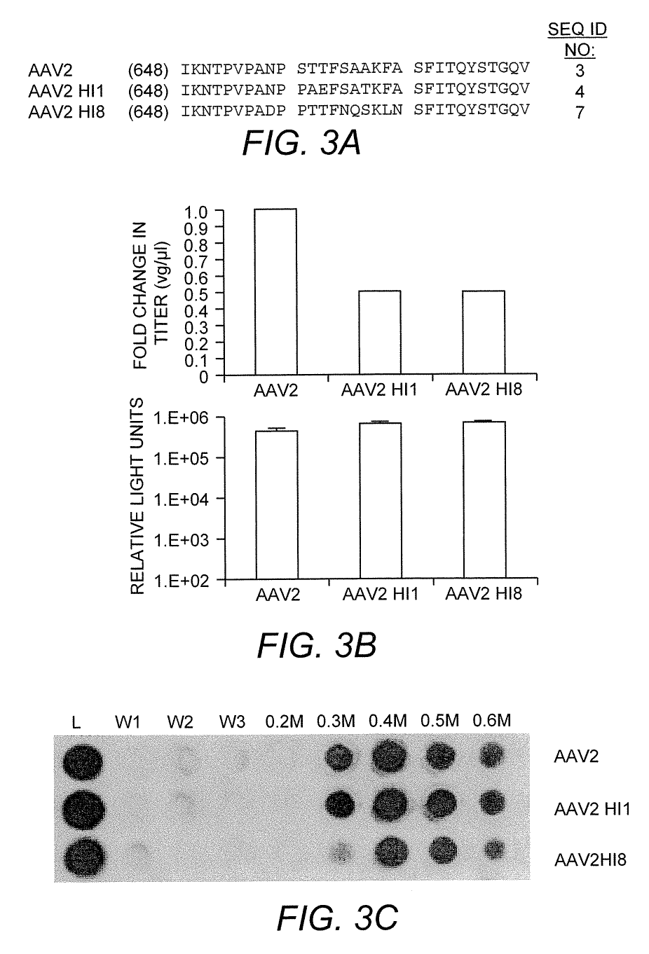

HI loop domain swapping. Based on the observations from the glycine residue replacement studies, we carried out HI loop swaps from representative serotypes in hopes of obtaining more information on critical amino acids that have evolved for the HI loops of specific serotypes. The serotypes chosen for this study were AAV1, AAV4, AAV5 and AAV8 which are 83%, 61%, 59% and 83% identical to the overall amino acid sequence of the AAV2 capsid, respectively (FIG. 1A), and represent the range of sequence homology between AAV2 and the other serotypes characterized to date (Gao et al. (2004) J Virol 78:6381-6388). The AAV1 and 8 HI loops are similar in conformation (data not shown) but vary significantly in amino acid sequence (FIG. 3A). AAV2 with the loops from AAV1 (AAV2 HI1) or AAV8 (AAV2 HI8) generated titers only two-fold lower than wt AAV2 at 3E9 vg/μl, 3E9 vg/μl and 6E9 vg/μl, respectively (FIG. 3B) and display similar to wt AAV2 heparan sulfate elution profiles (e.g. mostly at 0.4M PBS; FIG. 3C). As determined through EM analysis, the full particles obtained for the mutant seem to be similar to wt AAV2 full particles in gross conformation (data not shown). In addition, as seen in FIG. 3B AAV2 HI1, AAV2 HI8 and AAV2 all transduced 293 cells (infected with 3000 vg/cell) with similar efficiency as determined through a luciferase assay.

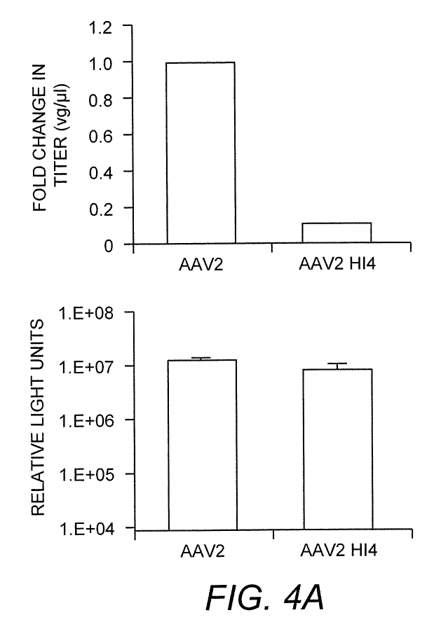

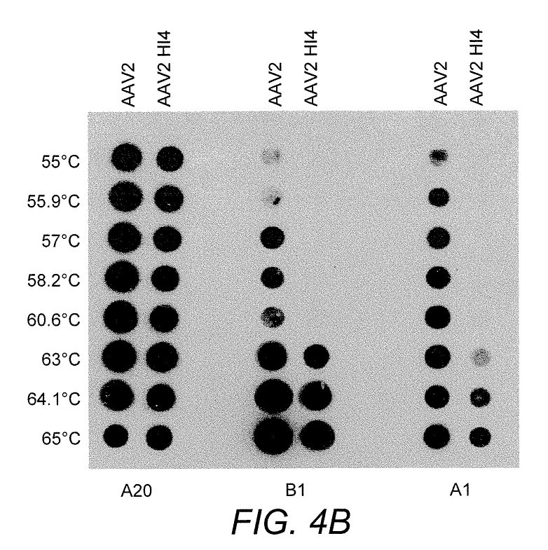

At the other end of the phenotype spectrum, swapping of the HI loops from the less homologous serotypes AAV4 and AAV5 did not produce mutant viruses that were similar to wt AAV2. As shown in FIG. 4A, AAV2 HI4 based on dot blot analysis, produced a virus titer one log lower than wt AAV2, 1.05E9 vg/μl and 1.13E8 vg/μl, respectively. However, when 293 cells were infected with the same number of vector genomes per cell, AAV2 HI4 and wt AAV2 infected cells with similar efficiency (FIG. 4A). Interestingly, when wt AAV2 and AAV2 HI4 full particles were heat treated at one-degree increments ranging from 55° C. to 65° C., AAV2 capsid dissociation was initiated at temperatures as low as 55° C., with complete dissociation at 63° C., whereas, AAV2 HI4 did not dissociate and expose VP1 until 63° C. (FIG. 4B). This observation suggests that the, AAV2 HI4 mutant capsid is more stable than that of wt AAV2, although we are still unsure if there is a correlation between capsid stability and titer.

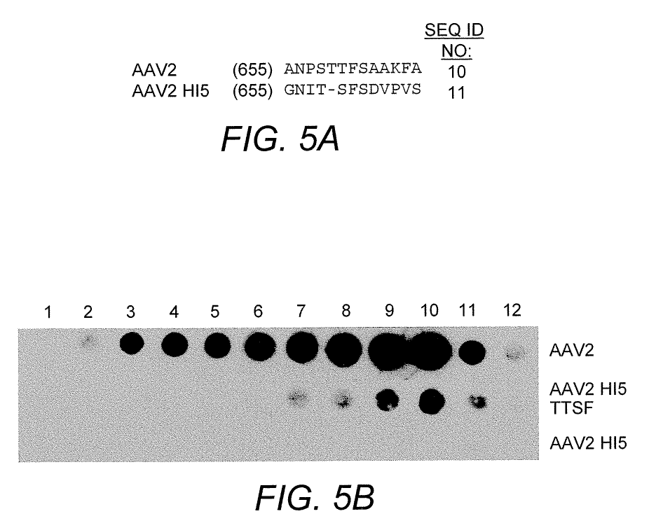

The AAV5 capsid VP subunit, with 59% homology in amino acid sequence to AAV2, contains an HI loop that is one amino acid shorter than those observed in AAV2 and the other AAV serotypes characterized to date. The AAV5 HI loop is structurally lacking a threonine at the amino acid position equivalent to residue 659 in AAV2. Substitution of this HI loop into AAV2 (AAV2 HI5) resulted in a change in amino acid sequence beginning at position 655 instead of 658 due to the low homology (FIG. 5A). Although AAV2 HI5 can express VP1, 2 and 3 when the cell lysate is subjected to western blot analysis (data not shown) the subunit proteins are unable to assemble into intact capsid particles as determined by A20 western dot blot (FIG. 5B). In order to determine if the length of the HI loop is contributing to the inability of AAV2 HI5 to assemble particles, a threonine was inserted into AAV2 HI5 at position 659 (AAV2 HI5 TTSF). The threonine insertion does minimally restore capsid assembly but not packaging based on western dot blot (FIG. 5B) and DNA dot blot analyses, respectively.

The HI loop swap phenotypes show that specific amino acid side chain interactions of the HI loop can affect particle stability as seen with the AAV2 HI4 mutant, and the length of the HI loop appears to be a factor in maintaining proper capsid assembly as in AAV2 HI5; however DNA packaging ability seems to be more stringently controlled by loop sequence as concluded from the AAV2 poly-glycine mutant. In addition, the data shows that this capsid VP region can tolerate amino acid differences in assembling an AAV capsid seen in AAV2 HI1 and HI8, consistent with the observation of the HI loop in all the parvovirus structures determined to date (FIG. 1A) despite the lack of sequence similarity.

Site-directed mutagenesis. To determine which amino acids within the HI loop are involved in viral genome packaging into the assembled capsids, a series of site-directed mutants were generated in this region based on sequence conservation between the AAV serotypes and observed interactions with the underlying amino acids, since replacing the AAV2 HI loop with glycines appears to ablate DNA packaging capabilities. As shown in the serotype sequence alignment in FIG. 1A, a phenylalanine at position 661 within the HI loop is conserved in the AAV serotypes aligned. In the crystal structure of AAV2 the side chain of F661 interacts with a conserved proline at position 373 in the EF loop within the underlying subunit possibly through Pi stacking (data not shown). Such stacking interactions have been shown to play key roles in protein stability and folding (Crespo et al. (2004) Eur J Biochem 271:4474-4484). Also seen in the sequence alignment, residue K665 is present in most serotypes (FIG. 1A), which based on the crystal structure forms a salt bridge with an aspartic acid at position 368 of the underlying subunit. Another amino acid of interest is F666, which resides in a hydrophobic pocket of the underlying subunit. As seen in the sequence (FIG. 1A) and structure alignment (data not shown), there is a hydrophobic residue such as valine or isoleucine at this position in all serotypes compared. In order to determine which of these three conserved residues are important for the AAV2 capsid to fully assemble and package the viral genome, the glycine residues at position 661, 665 and 666 in the AAV2 poly-glycine mutant were individually changed back to the amino acids present in the wt AAV2 HI loop, F661, K665 and F666, respectively. Mutating the residues one at a time did not restore the ability of the AAV2 poly-glycine mutant to package the viral DNA (Table 2). This suggests that cooperative interactions facilitated by individual residues maintain viral genome packaging capabilities, as seen in the AAV2 HI loop swap mutants (Table 2). This conclusion is further substantiated by the experiments mentioned below.

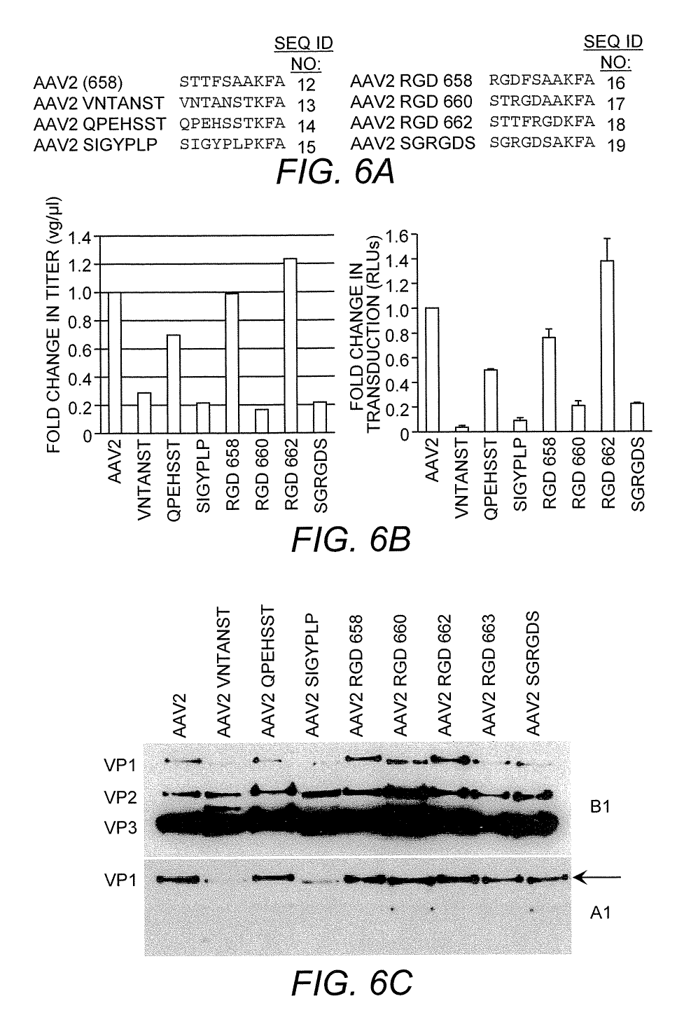

Peptide substitution studies. In order to determine the plasticity of the HI loop, gross mutagenesis of amino acid residues within this region was carried out on the capsid. Since data mentioned previously suggests a cooperative effect between amino acids is involved in viral genome packaging, semi-conserved residues K665 and F666 were left unchanged. The AAV2 HI loop was substituted with a range of peptide sequences varying in length and beginning at different amino acid positions. First, short RGD peptides were substituted, in an effort to “walk through” the HI loop and characterize the effects of disparate non-AAV sequences on viral assembly and packaging. Amino acid positions 658-660, 660-662, 662-664, and 663-665 were substituted with an RGD peptide (Shi and Bartlett. (2003) Mol Ther 7:515-525) (AAV2 RGD 658, AAV2 RGD 660, AAV2 RGD 662 and AAV2 RGD 663, respectively) (FIG. 6A). Most mutants were obtained at virus titers within two-fold of wt AAV2 with the exception of mutant AAV2 RGD 660, which was obtained at a six-fold lower titer than wt AAV2 at 1.48E8 vg/μl compared to 8.81E8 vg/μl (FIG. 6B). AAV2 RGD 658 and AAV2 RGD 662, after adjusting for vector genome number, resulted in similar infectivity to wt based on a luciferase assay (Table 2). However, when 293 cells were infected with AAV2 RGD 660, it was five-fold less infectious than wt AAV2 (FIG. 6B). RGD 660 was lower in titer and infectivity than wildtype, and results in the substitution of the conserved F661 (see Site-directed mutagenesis above) with a glycine residue, suggesting a potential role of F661 in the virus life cycle. In light of this single amino acid and its phenotypic effects, we introduced longer peptides into the AAV2 HI loop for increased amino acid variability, and to gain more insight into structure-function constraints in manipulating this region.

Seven amino acid peptides, successfully used as insertions in previous capsid mutagenesis studies, were chosen in order to determine if the variable region of the HI loop could tolerate these peptides as substitutions. Starting at position 658 in the AAV2 HI loop, we substituted with peptides QPEHSST, VNTANST, SIGYPLP (Work et al. (2006) Mol Ther 13:683-693). and SGRGDS (Koivunen (1993) J Biol Chem 268:20205-20210) (FIG. 6A). All mutants generated virus. AAV2 QPEHSST was able to make virus in titers similar to that of wt (within 2-fold) followed by AAV2 VNTANST (3.5-fold lower than AAV2), and AAV2 SIGYPLP and AAV2 SGRGDS (4.5-fold lower than AAV2). However, AAV2 VNTANST was 27-fold less infectious than wt AAV2 while, AAV2 SIGYPLP was reduced by approximately 10-fold in infectivity (Table 2). AAV2 SGRGDS and AAV2 QPEHSST were 4.5 and 2-fold less infectious than wt AAV2, respectively (FIG. 6B). The aforementioned changes in titer and infectivity upon gross mutagenesis of the AAV2 HI loops suggest that the amino acid interactions between the HI loop and underlying subunit are crucial for maintaining AAV viability. Previous data in the literature suggests that the five-fold axis is important for viral genome packaging and VP1 externalization (Bleker et al. (2005) J Virol 79:2528-3540; Kronenberg et al. (2005) J Virol 79:5296-5303; Wu et al. (2000) J Virol 74:8635-8647). We do see a viral genome packaging defect in some of the mutants mentioned above, correlating with a decrease in infectivity. To further understand the phenotypic changes observed, we carried out a battery of biochemical analyses.

Biochemical analysis. A series of biochemical analyses such as heparin binding, heat treatment and western blotting were performed in order to understand why the titer and infectivity of AAV2 RGD 660, AAV2 VNTANST, AAV2 SIGYPLP and AAV2 SGRGDS were consistently lower than wt AAV2, from such studies, western blot analysis revealed an interesting capsid phenotype as shown in FIG. 6C. We used monoclonal antibodies B1 and A1 (Wobus et al. (2000) J Virol 74:9281-9293) that recognize the VP3 C-terminus and VP1 unique N-terminus, respectively, to characterize the peptide substitution variants described above. Based on B1 staining, AAV2 VNTANST had decreased VP1 incorporation and an extra protein band between VP2 and VP3. In addition, AAV2 SIGYPLP had decreased VP1 incorporation. Interestingly, when blotted with A1 antibody against the VP1 unique region of AAV2, AAV2 QPEHSST, AAV2 RGD 660, and AAV2 SGRGDS revealed a second band at 77 kDa. Notably, AAV2 RGD 660 and the longer peptide substitutions that were detrimental to virus titer and infectivity all changed the amino acid type at the conserved phenylalanine at position 661 (data not shown). Therefore the role of this residue in the AAV2 HI loop functionality was further investigated.

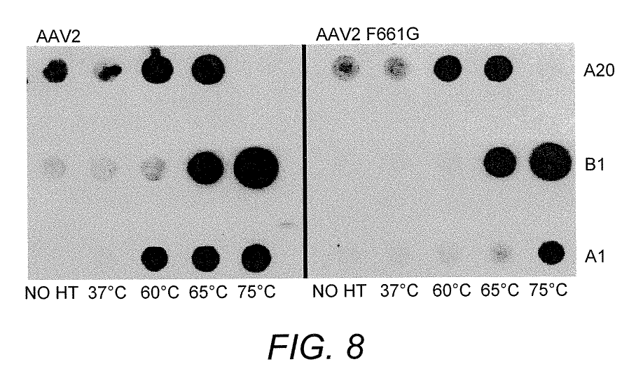

Analysis of the conserved F661 residue. F661, which is completely conserved throughout all AAV serotypes (FIG. 7A) and interacts with a proline in the underlying VP subunit, was mutated to a glycine residue in AAV2 (AAV2 F661 G). AAV2 F661 G produced virus five-fold lower in titer than wt AAV2 based on dot blot analysis, 8.2E7 vg/μl and 4.1E8 vg/μl respectively (FIG. 7B). AAV2 F661 G also binds heparin with a similar affinity as wt AAV2 based on heparin column binding and elution with increasing concentrations of salt (Table 2). AAV2 F661G is one log less infectious than AAV2 based on a luciferase assay (FIG. 7C). In addition to these data, AAV2 F661 G and wt AAV2 capsids were heat treated at 37° C., 50° C., 60° C., 65° C. and 75° C. While, AAV2 was able to expose the VP1 N-terminus based on A1 antibody staining at 60° C., AAV2 F661 G is unable to do so at this temperature, and can only expose the VP1 N-terminus when heated to 75° C. upon capsid dissociation (FIG. 8). To further corroborate the data showing decreased incorporation of VP1 and reduced infectivity of AAV2 F661 G, we carried out intracellular trafficking studies using confocal fluorescence microscopy. Briefly, Hela cells were infected with 30,000 vg/cell of wt AAV2 and AAV2 F661G particles. The cells were fixed 12 hrs post infection and stained with primary A20 antibody for intact capsid detection and then secondary goat-anti mouse 488 nm-fluorophore conjugated antibody. In addition, the nuclei were stained with DAPI. Based on this analysis, AAV2 F661G was unable to enter the nucleus efficiently and appeared to remain perinuclear, unlike wt AAV2, which trafficked into the nucleus more efficiently (data not shown).

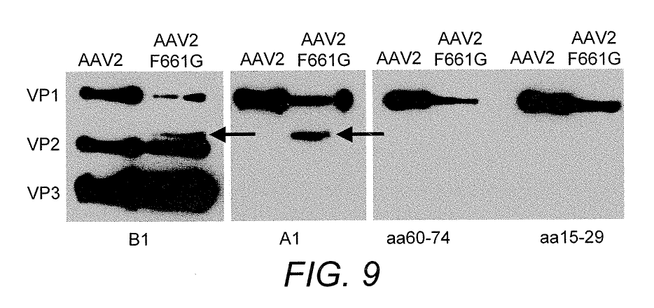

In addition, when 1E9 vg dialyzed full particles were run on a western blot and stained with monoclonal B1 and A1 antibodies, AAV2 F661G revealed a fourth molecular weight capsid species between VP1 and VP2 consistently running at 77 kDa. Using antibodies that specifically detect amino acids 15-29 and 60-74 in the VP1 unique region, the capsid band at 77 kDa was not detected on the western blot, further confirming an N-terminal truncation of this capsid subunit (FIG. 9). This molecular weight species is identical to the novel protein band seen in the western blot with AAV2 QPEHSST, AAV2 RGD 660 and AAV2 SGRGDS capsid subunit proteins. The novel capsid subunit is approximately 100 amino acid residues shorter than VP1, implicating potential proteolytic processing of the exposed VP1 N-terminus in these HI loop variants.

Therefore, the aforementioned data demonstrates that the HI loop can tolerate most amino acid changes, and specific cooperative amino acid interactions are necessary for proper viral genome packaging. Additionally, the F661/P373 hydrophobic interaction facilitates proper incorporation of the VP1 subunit into the AAV2 capsid. Without the proper interactions a distinct VP1 subunit lacking its N-terminus containing the phospholipase activity is incorporated into the capsid, directly impacting virus infectivity.

With the availability of the AAV2 crystal structure (Xie et al. (2002) Proc Natl Acad Sci USA 99:10405-10410), many aspects of the adeno-associated virus life cycle including host cell recognition (Akache et al. (2006) J Virol 80:9831-9836; Di Pasquale et al. (2003) Nat Med 9:1306-1312; Kern et al. (2003) J Virol 77:11072-11081; Qing et al. (1999) Nat Med 5:71-77; Walters et al. (2001) J Biol Chem 276:20610-20616), intracellular trafficking (Bartlett et al (2000) J Virol 74:2777-2785; Ding et al. (2005) Gene Ther 12:873-880) and uncoating (Thomas et al. (2004) J Virol 78:3110-3122) are now possible to correlate to structure. The first of such studies have centered around the three-fold loops and the determination that they are key topological features in host cell recognition (Asokan et al. (2006) J Virol 80:8961-8969; Kern et al. (2003) J Virol 77:11072-11081). Similar structure-function studies have extended from the three-fold loops to the five-fold axis and the location of the virion pore and its potential role in viral genome packaging, capsid assembly and VP1 unique N-terminal exposure (Bleker et al. (2005) J Virol 79:2528-3540; Kronenberg et al. (2005) J Virol 79:5296-5303; Wu et al. (2000) J Virol 74:8635-8647. Interestingly, the HI loop surrounds the five-fold pore, and has a structural role in viral assembly by overlapping the neighboring VP3 subunit forming amino acid interactions with the underlying EF loop (FIG. 1 and data not shown). Recently Dr. Mavis Agbandje-McKenna has observed the HI loop flipping up 90° upon AAV2-heparan sulfate proteoglycan binding suggesting a dynamic role of this structure in the viral infectious pathway (unpublished). To better understand the role of this capsid structure, we chose to characterize in detail the HI loop as it may contribute to specific stages in the virus life cycle such as viral genome packaging, assembly, and subsequent stages during the infectious pathway. The results of this study demonstrate that the AAV2 HI loop is involved in proper capsid assembly, packaging of the viral DNA, and viral infectivity when the conserved phenylalanine at amino acid position 661 is altered.

We carried out a comprehensive amino acid deletion and substitution study to uncover the role of the HI loop in the AAV life cycle. From these efforts we determined that removal of the HI loop (AAV2 HI−/−) leads to capsids that cannot assemble (FIG. 2B). We assayed viral assembly primarily using the monoclonal A20 antibody (Wobus et al. (2000) J Virol 74:9281-9293) that detects tertiary structure of properly assembled AAV capsids. Viruses that were unable to form virions were further studied using gradients and western blot analyses that confirmed the ability to synthesize viral protein subunits (data not shown). Although we relied primarily on A20 recognition to confirm the ability to form proper virion structures, additional studies such as iodixanol gradient purified AAV2 HI−/− cell lysate followed by EM analysis further determined that this mutant only appears to generate monomer subunits (data not shown). In contrast, substitution of the loop with glycines (AAV2 poly-glycine) generated A20 recognizable assembled capsids, however these capsids were deficient in the ability to package the viral DNA. Even though this mutant provided sufficient structure to assemble intact AAV particles, the glycine substitutions specifically ablate amino acid side-chain interactions with the EF loop of the underlying subunit, suggesting that the HI loop structure and the backbone interactions of the HI loop with the underlying subunit are sufficient enough to facilitate capsid formation. However, the specific amino acid interactions are important for efficient packaging of the viral DNA. Although we cannot draw from our studies the exact mechanism for the viral genome packaging deficiencies of the glycine substitution mutant, it is interesting to speculate that this phenotype can possibly be attributed to gross conformational changes in the structure of the five-fold pore since the five-fold pore has been implicated as the site of rep protein binding, a necessary step for efficient DNA encapsidation (Bleker et al. (2006) J Virol 80:810-820; Bleker et al. (2005) J Virol 79:2528-3540).

In addition to the deletion and substitution studies above, we decided to swap the AAV2 HI loop with those from representative serotypes. Domain swapping, specifically between virus serotypes allows for determination of the role of that specific region of the capsid in the virus life cycle. Interestingly, in our studies, swapping the HI loop with that of AAV1 and AAV8 did not affect titer, transduction, heparin binding or gross conformation. However, this can be expected due to the relatively higher sequence homology between AAV2 and these serotypes. More importantly, these results suggest that the HI loop most likely does not contain determinants of tissue tropism or receptor binding. This was further confirmed via mouse intramuscular injections with AAV2 RGD 662. Briefly, bioluminescence imaging revealed similar in vivo transduction profiles for AAV2 RGD 662 and wt AAV2 one-week post administration (data not shown).

In the case of AAV4 and AAV5 HI loops, significant phenotypic variations were seen possibly due to lower sequence homology with AAV2. For instance, the AAV4 HI loop is comprised of a higher number of hydrophobic residues than the loop from AAV2 based on the amino acid sequence and crystal structure (Govindasamy et al. (2006) J Virol 80:11556-11570). The 3-dimensional structure of the AAV2 VP3 monomer (data not shown) shows that the side chains of residues 659 and 660 point away from the capsid and do not interact with the residues in the underlying subunit. On the other hand, the conserved phenylalanine at position 661 interacts with proline 373 in the EF loop of the underlying subunit. The alanine-to-serine change at position 663 of the AAV2 HI4 mutant might contribute a hydrogen bond interaction due to a potentially accessible hydroxyl group that is not present in the AAV2 HI loop. The K665P change in AAV2 HI4 suggests a possible contribution to increased hydrophobic interactions with proline, valine, phenylalanine and methionine residues found in the underlying subunit of AAV2. However, this assessment is based on a structure model of AAV2 HI4 and a more accurate analysis of the AAV4 HI loop amino acid contributions to AAV2 capsid stability is dependent upon knowing the crystal structure of the AAV2 HI4 mutant.

Collectively, the aforementioned amino acid changes in AAV2 HI4 could enhance HI loop-EF loop interactions and thereby could well account for increased capsid stability as demonstrated through increased resistance to heat treatment in comparison to AAV2. In addition, such increases in capsid stability and possible gross conformational changes to the five-fold pore might account for lower packaging efficiency and titers seen with the AAV2 HI4 mutant. It is possible that the AAV capsid “breathes” or expands in volume during viral genome packaging, and if the capsid is too stable or held too tightly together, it may be more difficult for the rep protein to package the viral genome. The idea of capsid expansion has been studied in bacteriophage, and it has been shown that during the DNA packaging process a conformational change occurs which causes an increase in capsid volume (Jardine and Coombs (1998) J Mol Biol 284:661-672).

Additionally, previous data suggest that the rep protein is bound in higher quantities to the capsids of packaging deficient mutants (Bleker et al. (2006) J Virol 80:810-820) possibly due to “jamming” of the genome threading machinery. Such has also been noted in the case of AAV2 HI4, wherein the particle bound increased amounts of rep protein in comparison with AAV2 (data not shown). For the AAV2 HI4 mutant, it is possible that there is increased stability of the particle based on the presence of another protein or proteins on the capsid surface.

In the case of AAV2 HI5, despite normal expression of capsid subunit proteins, no intact capsid assembly is observed. This was further confirmed via EM analysis on cesium chloride gradient fractions of the AAV2 HI5 transfected cell lysate. It was determined that the AAV2 HI5 mutant may form pentamers, but does not form proper intact particles (data not shown). This phenotype is likely attributable to the fact that the AAV5 HI loop is one amino acid shorter, based on the crystal structure (Walters et al. (2004) J Virol 78:3361-3371) than the wt AAV2 HI loop. In corollary, insertion of the missing threonine at position 659, minimally rescues capsid assembly. Therefore, the length of the HI loop in relation to the underlying subunit appears to be a factor in proper capsid assembly, while the loop sequence dictates genome packaging efficiency. This was further corroborated by the fact the AAV2 HI loop extensions (data not shown) formed intact virus particles, but were unable to package the viral genome.

Following the domain swaps we decided to use peptide substitutions in order to mutate multiple residues of the HI loop. Many groups have successfully inserted peptides, specifically at the three-fold loops, on the capsid surface as a means to retarget the virus for specific tissue types (Girod et al. (1999) Nat Med 5:1052-1056; Shi and Bartlett. (2003) Mol Ther 7:515-525; Shi et al. (2006) Hum Gene Ther 17:353-361; Work et al. (2006) Mol Ther 13:683-693). In this study we used peptides, not as insertions, but substitutions in a novel region of the capsid. Peptide substitution within the AAV2 HI loop showed that certain amino acid changes do not affect virus titer and transduction, as seen with the AAV2 RGD 658, AAV2 RGD 662 and AAV2 QPEHSST mutants. However, some peptide substitutions resulted in marked changes in phenotype that were dependent on the amino acid position substituted. For instance, substitution with peptide RGD at position 660 (AAV2 RGD 660), SGRGDS starting at position 658 (AAV2 SGRGDS), VNTANST starting at position 658 (AAV2 VNTANST) and SIGYPLP also starting at position 658 (AAV2 SIGYPLP) resulted in decreased titer and infectivity. All of these mutants replace the conserved F661 residue observed in all serotypes.

Interestingly, a number of these mutants, such as AAV2 VNTANST, SIGYPLP, RGD 660 and SGRGDS, also revealed differential banding patterns seen with B1 antibody staining of a western blot (FIG. 6C). Specifically, there appears to be a decreased incorporation of VP1 capsid subunits in these mutant capsids. It is likely that such phenotype, which would reduce the effectiveness of the PLA2 domain (located in the VP1 N-terminal domain) required for endosomal escape and nuclear entry of the viral capsid, could explain the decrease in transduction seen with these mutants (Girod et al. (2002) J Gen Virol 83:973-978; Grieger et al. (2007) J Virol 81:7833-7843; Kronenberg et al. (2005) J Virol 79:5296-5303; Sonntag et al. (2006) J Virol 80:11040-11054). The lower titers of the aforementioned mutants can possibly be attributed to improper capsid assembly (Bleker et al. (2006) J Virol 80:810-820; Timpe et al. (2005) Curr Gene Ther 5:273-284) and defective packaging (Bleker et al. (2006) J Virol 80:810-820). In the AAV2 VNTANST mutant there is an additional protein band seen between VP2 and VP3 with B1 staining that is yet to be identified. The observed protein product is most likely due to proteolytic processing of VP1, which could also account for the decreased amount of VP1 present in this capsid mutant. The 77 kDa protein band in the case of AAV2 RGD 660 and AAV2 SGRGDS seen with A1 staining further corroborates these speculations (FIG. 6C).

As mentioned above one commonality shared by these defective peptide substitution mutants is that they span the conserved phenylalanine at amino acid position 661. F661 interacts with P373 in the EF loop in the underlying subunit of all serotypes through stacking interactions (data not shown). This interaction appears to be involved in stability (Crespo et al. (2004) Eur J Biochem 271:4474-4484) of assembled capsid subunits since the HI loop is the only region at the five-fold axis of symmetry that extends from one subunit and overlaps the underlying subunit. Mutation of F661 results in a phenotype similar to that seen with peptide substitutions spanning this region. Based on data shown in FIG. 9, it appears that the interaction between F661 and P373 stabilizes the capsid around the five-fold axis of symmetry, the latter which contributing to viral genome packaging and infectivity (Bleker et al. (2005) J Virol 79:2528-3540; Wu et al. (2000) J Virol 74:8635-8647). Disruption of this interaction appears, in particular, to reduce the amount of VP1 incorporated into these mutant capsids.

Additionally, such mutagenesis could result in improper incorporation of VP1 subunits at the five-fold axis of symmetry, which would expose the PLA2 domain to cellular proteases during virus production. If unassembled VP1 monomers or loosely assembled particles exposing the VP1 unique N-terminus are present, it is possible that they may be susceptible to cellular proteases. This may not occur as readily in wildtype or other mutant viruses that are able to assemble the VP monomers efficiently in a stable configuration. In conjunction with this observation, a similar phenomenon may be occurring in the AAV baculovirus production system (Sollerbrant et al. (2001) J Gen Virol 82:2051-2060; Urabe et al. (2002) Hum Gene Ther 13:1935-1943). There appears to be inefficient incorporation of VP1 into the AAV2 capsid during production in insect cells, and this may be due to the susceptibility of VP1 to cellular protease in the non mammalian cell environment (Kohlbrenner et al. (2005) Mol Ther 12:1217-1225). The notion that VP1 is susceptible to cellular proteases is further substantiated by the fact that when mammalian cells are transfected with VP1 constructs, specifically VP1NLSFKN and VP1 FKN, a second molecular weight band is detected between VP1 and VP2 in the cell lysates (Grieger et al. (2007) J Virol 81:7833-7843) similar to the result obtained in this study. Upon mutation of F661 this molecular weight species was not only generated but also incorporated into the intact capsid.

In addition, it is not surprising that a single amino acid on the AAV capsid such as F661 could significantly impact the biology of the virus. A recent study from our lab has shown that a single amino acid mutation, specifically K531E in AAV6 and E531K in AAV1, suppresses and enhances heparin binding, respectively (Wu et al. (2006) J Virol 80:11393-11397). Taken together, our data supports the role of the HI loop as an important AAV capsid structural element, necessary for proper incorporation of VP1 into an assembled infectious particle and a functional five-fold pore that allows efficient packaging of viral genomes.

### Example 2

### Further Characterization of AAV with Mutant HI Loops

### Summary

The ability to manipulate the adeno-associated virus capsid allows researchers to understand capsid domains and develop efficient vectors for gene delivery. In this report we have focused on the HI loop, a previously characterized capsid domain. We substituted specific amino acids, AAV2 aa662-667, located within this region of AAV2 and AAV9 capsids with a hexa-histidine tag. The resulting AAV2 HI6× His and AAV9 could be purified via nickel affinity chromatography as a potential universal purification scheme for AAV vectors. Further we were able to conjugate Ni-NTA gold nanoparticles to these capsids, thereby demonstrating their potential as novel reagents for EM applications. While the presence of hexa-histidine tags on the surface did not affect capsid infectivity in vitro, significantly reduced transduction was observed following intramuscular administration in mice. Interestingly, His-tagged AAV capsids were detargeted from the liver following intravenous administration. However upon generation of chimeras with fewer hexa-histidine tags on the surface, transduction is rescued in vivo. In summary, we have introduced a multifunctional domain into a novel site on the capsid surface that can be utilized for universal AAV vector purification, capsid labeling with gold nanoparticles and generating vectors detargeted from the liver.

### Generation of Mutants

AAV2 HI6× His mutants were generated using PCR with the Expand Long Template PCR kit (Roche) or site directed mutagenesis (Stratagene QuikChange). Primers complementary to the pXR2 backbone were generated by Integrated DNA Technologies (www.idtdna.com) with nucleotide extensions coding for the histidine tag. The primers used for AAV2 HI6× His were 5′-CACCATCACCATCACCATTCCTTCATCACACAGTACTCCACGGGACAG-3′ and 5′-ATGGTGATGGTGATGGTGGAAGGTGGTCGAAGGATTCGCAGGTAC-3′ (PCR Expand Long Template) Amino acids 662-667 in VP3 were replaced with the histidine residues. For the mutants containing fewer histidine residues within the HI loop primers were designed as follows for use in site-directed mutagenesis: AAV2 HI1 His: 5′-GAATCCTTCGACCACCTTCAGTCACGCAAAGTTTGCTTCCTTC-3′, AAV2 HI2His: 5′-CCTGCGAATCCTTCGACCACCTTCAGTCACCACAAGTTTGCTTCC-3′, AAV2 HI3His: 5′-CCTTCGACCACCTTCCACCACCACAAGTTTGCTTCCTTCATCACACAG-3′, AAV2 HI4His: 5′-CCTGCGAATCCTTCGACCACCTTCCATCACCACCACTTTGCTTCCTTC-3′, AAV2 HI5His: 5′-GCGAATCCTTCGACCACCTTCCATCACCACCACCACGCTTCCTTCATCAC-3′.AAV9 HI6× His was generated by GeneArt.

The nucleic acid and amino acid sequences of the AAV2, AAV2 HI6× His, AAV9 and AAV9 HI6× His VP1 capsid proteins are shown in Table 4.

### Virus Production

Virus was produced using the triple transfection method described in Xiao et. al. (J. Virol. 72:2224-32 (1998)). Cells were transfected with pXR (pXR2 or pXR9) containing the capsid mutations, pXX6-80 helper plasmid, and pTR-CMV- or CBA-Luciferase. In the case of chimeric mutants, pXR2 and pXR2 HI6× His was transfected at a ratio of 1:1 or 5:1, respectively, totaling 10 ug plasmid DNA (Rabinowitz et al., J. Virol. 78: 4421-32 (2004)). Cells were harvested 60 hrs post transfection and purified using cesium chloride gradient density centrifugation for 5 hrs at 65,000 rpm or overnight at 55,000 rpm for iodixanol gradient centrifugation. CsCl gradients were fractionated and virus dialyzed against 1×PBS with calcium and magnesium, or the layer between 40 and 60% iodixanol was pulled from the gradient and further purified on the FPLC 1 ml His-Trap HP nickel column (Amersham). Viral titers were determined by dot blot analysis or qpcr using primers specific to the TR-Luciferase transgene.

### Fast Protein Liquid Chromatography

Various volume and vector genome quantities of iodixanol gradient purified virus was further purified through a nickel column (HisTrap HP 1 ml column, Amersham) at a flow rate of 0.2 ml/min. Load volumes were mixed with binding buffer (5 mM imidazole, 20 mM sodium phosphate and 0.5M NaCl pH 7.4)) prior to loading. The column was washed with 5 column volumes water, equilibrated with 5 column volumes binding buffer, 5 ml of sample mixed with binding buffer was injected into the column, washed with 5 column volumes binding buffer, and eluted with 5 volumes elution buffer (500 mM imidazole, 20 mM sodium phosphate and 0.5M NaCl pH7.4). Peak fractions were detected based on UV absorbance at 280 nm and verified by vector genome quantification using qpcr with primers specific to the luciferase transgene. Peak fractions were dialyzed against 1×PBS Ca++Mg++.

### In Vitro Transduction Assays

293 cells were infected with 1000 to 3000 vector genome containing particles of AAV2 HI6× His per cell pre and post FPLC column elution. Cells were lysed with 100 ul 1× passive lysis buffer (Promega) and the lysates were mixed with 100 ul luciferin (Promega). Relative light units were detected with a Victor2 Luminometer.

### Gold Particle Conjugation

Approximately 1E9 virus particles (AAV2 or AAV2HI6× His eluted from the nickel column) were mixed with Ni-NTA-nanogold particles at a concentration 60 times the nanomolar amount of His tags present in the sample. The virus particles were blocked with 0.05% Tween, 20 mM NaCl and 50 mM Tris pH7.5 for 30 mins at room temperature. The gold particles were added to the blocked virus particles and incubated anywhere from 5 to 30 mins room temperature.

### Electron Microscopy

Glow discharged copper grids were incubated with 15 ul virus or virus pre-incubated with gold particles for 2 minutes. For virus not conjugated to gold particles, the grids were washed twice with ddH2O and then negatively stained with 2% uranyl acetate stain for 30 seconds. For virus conjugated to gold particles, grids were washed 2× with wash buffer containing 200 mM NaCl and 20 mM imidazole to remove nonspecific binding. They were then washed with ddH2O and stained with Nanovan (Nanoprobes). Grids were visualized using the Leo 910 TEM in the Microscopy Laboratory Services at the University of North Carolina at Chapel Hill.

### In Vivo Experiments

BalbC female mice were injected via tail vein with 1E10 or 1E11 or intramuscular injection into the gastrocnemus with 1E10 vector genome containing particles (AAV2, AAV2 HI-His mutants, AAV9, AAV9 HI6× His). Mice were imaged at week intervals post injection following intraperitoneal injection with D-Luciferin (NanoLight) using the IVIS Xenogen imaging system. Mice were imaged for 1 minute or 5 minutes at 5 minutes post administration of D-luciferin substrate.

### Tissue Harvesting and Genome Quantification

Animals were sacked two weeks post IV injection. The liver was harvested and a section of the liver was homogenized and incubated with 180 ul ATL buffer and 20 ul proteinase K solution (Qiagen DNeasy Blood and Tissue Isolation Kit). The tissue was incubated at 50 degrees overnight and genomes in the tissue were quantified via qPCR (Roche) with primers specific to the luciferase transgene. The primers used were 5′-AAV AGC ACT CTG ATT GAC MA TAC-3′ and 5′-CCT TCG CTT CAA AAA ATG GAA C-3′. The mouse lamin B2 gene was also quantified and vector genome copies were normalized to the number of diploid genomes present in the sample.

### Results

As a gene therapy vector, AAV is under constant characterization and modification in hopes of developing a more efficient vector for gene therapy. To generate enhanced vectors, we focus on capsid structure-function relationships. Over the years research has shown that specific regions of the virus capsid are implicated in specific virus functions. For instance, the VP1 N-terminus for phospholipase activity necessary for virus trafficking (Sonntag et al., J. Virol. 80:11040-54 (2006)), basic regions for capsid assembly and nuclear localization (Grieger et al., J. Virol. 80:5199-210 (2006)), and the five-fold pore for viral genome packaging and potentially rep protein interactions and VP1 unique N-terminal externalization (Bleker et al., J. Virol. 79:2528-40 (2005)). Recently, it has been shown that the HI loop on the capsid surface is necessary for proper viral protein incorporation into the intact particle. Additionally, it was determined that the HI loop is highly plastic if specific amino acid interactions are conserved between the HI loop and the underlying subunit. Based on these structure-function studies, we demonstrated the plasticity of this novel capsid region, and its ability to tolerate amino acid substitutions.

Specifically, we substituted amino acids 662-667 with six histidines for tagged universal metal affinity purification of any AAV. There are multiple proposed purification techniques such as antibody purification and column chromatography (Koerber et al., Hum. Gene Ther. 18:367-78 (2007)), both of which are waiting expansion to other serotypes. Additionally there are reports of tagged purification methods involving insertion peptides at the VP2 N terminus or the heparin binding sited within VP3, both of which provide potential reagents for various applications. This study aids in the development of a universal purification method for all AAV serotypes, demonstrates the utilization of this domain as a site for gold particle conjugation, and displays a novel method for vector tropism alteration.

### AAV Hexa-Histidine Production and In Vitro Characterization

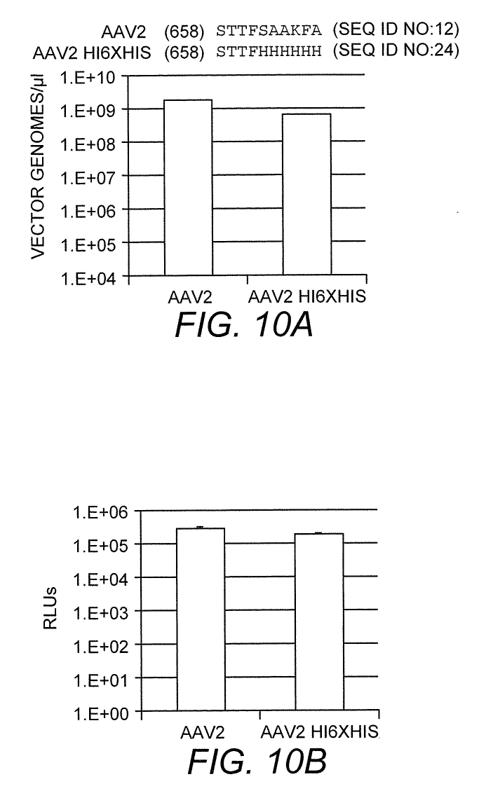

The HI loop, surrounding the fivefold pore, was chosen as the site of peptide substitution due to the plasticity of this region (see Example 1). Without affecting the conserved capsid stabilizing phenylalanine-proline interaction at amino acid position 661, residues 662-667 (VP1 numbering) of the AAV2 capsid were substituted with six histidine residues via PCR. The histidine residues were chosen for the development of a universal purification method applicable to any AAV. Virus particles generated contained 60 copies of the tag due to its presence in each VP3 monomer. Incorporation of the hexa-histidine motif into the virus capsid did not affect virus titer and transduction based on luciferase assay post infection of 3000 vector genome containing particles per 293 cell (FIG. 10). The hexa-histidine tag was also incorporated into the AAV9 capsid at the same location (GeneArt) for further purification characterization.

### Affinity Column Purification of AAV Hexa-Histidine Vectors

The hexa-histidine tag was tolerated by the AAV2 and AAV9 capsid, therefore, it was necessary to determine if these particles could bind a nickel column and be eluted from that column in a pure peak fraction. We first validated virus particle nickel-binding affinities with Micro Bio-Spin Chromatography Columns (BioRad) packed with nickel-agarose beads (Ni-NTA Qiagen). Beads were incubated with 1E10 vector genome containing particles (cesium chloride gradient purified). Flow through, wash and elution fractions (buffers from Qiagen Ni-NTA kit) were collected and blotted on a nitrocellulose membrane. AAV2 HI6× His bound the nickel beads and were eluted from the packed column, whereas AAV2 wildtype particles were unable to bind the column based A20 primary antibody staining detecting intact capsids on a nitrocellulose membrane (data not shown).

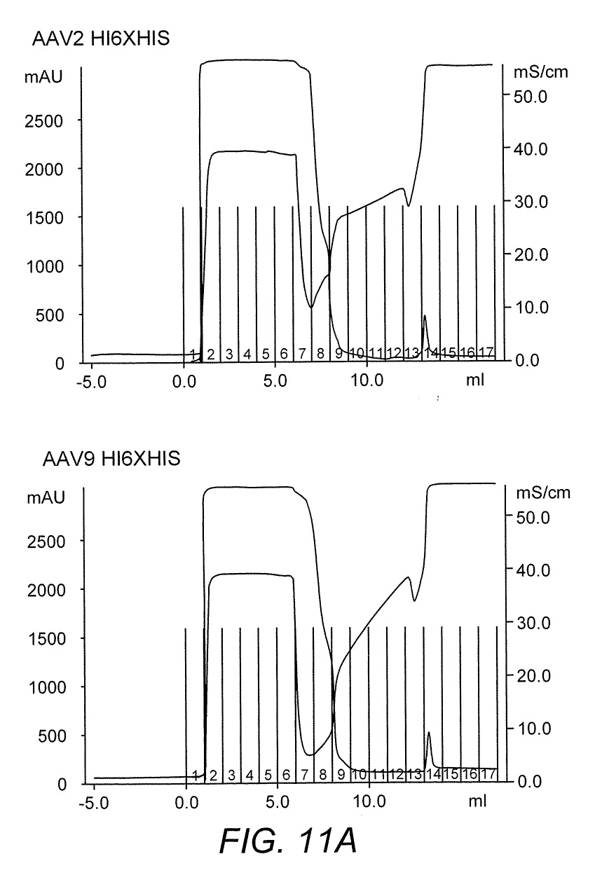

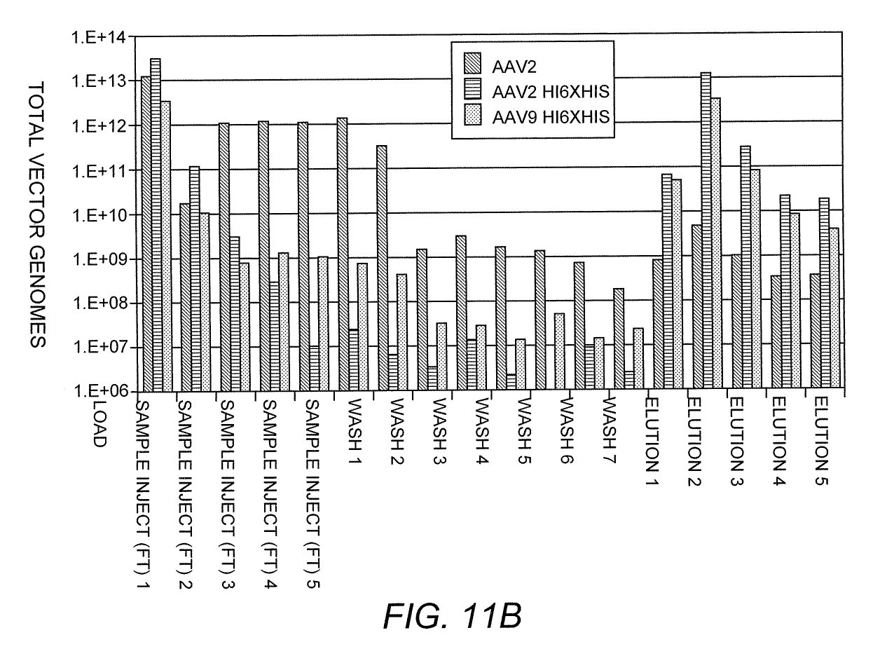

In order to further assess the nickel binding capabilities of the tagged virus, iodixanol gradient purified virus was loaded onto an FPLC nickel His-Trap HP column (Amersham). Following equilibration, approximately, 1E13 vector genome containing particles of AAV2 HI6× His were injected across the column. AAV2 HI6× His bound to the column and eluted primarily in a peak elution fraction based on FPLC protein detection at 280 nm (FIG. 11A). In order to confirm the elution of vector genome containing particles qPCR of the luciferase transgene was performed on each FPLC fraction (FIG. 11B). The protein detection in the peak elution fraction directly corresponded with the vector genomes recovered from that fraction. Therefore, viral particles remained intact during the elution from the nickel column. On the other hand, AAV2 wildtype particles did not bind to the column efficiently and primarily eluted in the column wash fractions based on the protein chromatogram and vector genome quantification (FIGS. 11A and 11B). To further substantiate the capability of the AAV particle containing the hexa-histidine tag to bind nickel, AAV9 HI6× His was passed through the nickel column. In corroboration with previous data, AAV9 HI6× His bound the nickel column and eluted off with 500 mM imidazole with a similar chromatographic and vector genome quantification profile as to AAV2 HI6× His (FIGS. 11A and 11B). Collectively, aforementioned results suggest potential development of a universal purification scheme for AAV vectors.

### AAV Hexa-Histidine Particles Purity

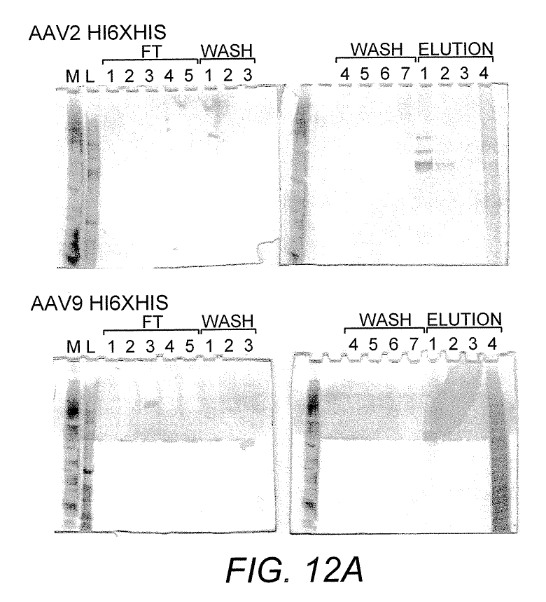

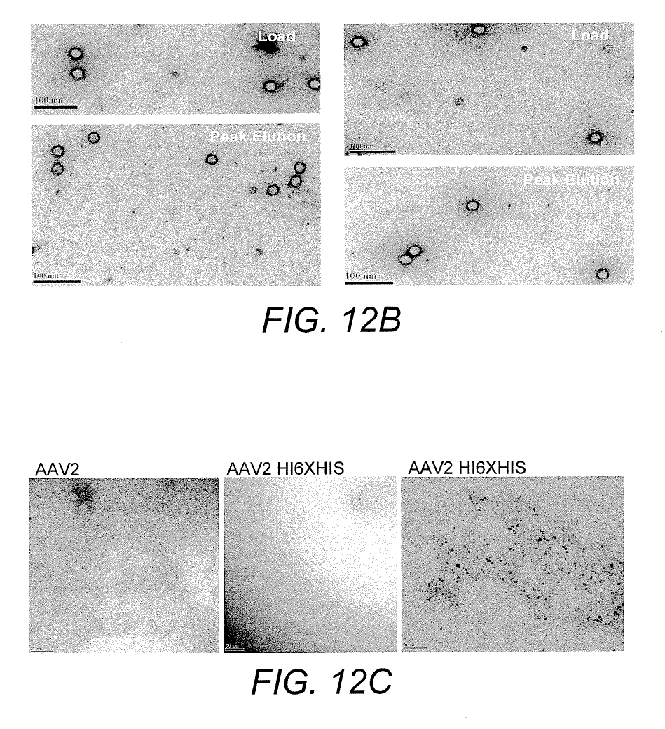

Additional biochemical analyses were performed in order to further characterize the purity and infectivity of the virus particles eluted from the FPLC nickel column. AAV2 HI6× His and AAV9 HI6× His FPLC column fractions (30 ul), including the load, were run on a 10% Bis-Tris acrylamide gel (NuPage, Invitrogen). Gels were silver stained with Invitrogen Silver Express and fraction 14, the peak elution fraction from the FPLC column runs involving AAV2 HI6× His and AAV9 HI6× His showed pure, concentrated viral protein bands of VP1, VP2 and VP3 as compared to the load control (FIG. 12A).

Using a Leo 910 TEM, electron micrographs were taken of the load and peak fractions of AAV2 HI6× His and AAV9 HI6× His FPLC column runs. EM images revealed increased purity of peak column fractions as compared to the load fractions (FIG. 12B). Not only did virus sample purity increase post nickel column purification, but also virus infectivity was maintained (data not shown). This data demonstrates that replacing amino acids 662-667 with a hexa-histidine tag is a valid method for AAV universal purification. The hexa-histidine peptide allows for recovery of a pure virus fraction post metal affinity purification (FIGS. 12A and 12B). Based on these data, we chose to characterize the virus in vivo.

### Hexa-Histidine Nanogold Labeling

In addition to the aforementioned studies, we evaluated the potential for exploiting the hexa-histidine tag on the AAV capsid for nanogold labeling in EM application. From Nanoprobes, Ni-NTA-nanogold of 1.8 nm in diameter was obtained and incubated in excess with AAV2 and AAV2 HI6× His virus particles. The virus particles were then stained on a copper grid. As expected the Ni-NTA-nanogold particles bound to AAV2 HI6× His containing the hexa-histidine tag but did not bind the wildtype AAV2 particles that are lacking the tag as detected by electron microscopy (FIG. 12C). Therefore, this domain is a novel site to use for gold particle conjugation, for example, for the purpose of labeling a viral particle as a means to characterize its properties in vitro, such as subunit detection, viral particle tracking, or detection in tissue samples for histological analysis.

AAV Hexa-Histidine Vectors are Detargeted from the Liver

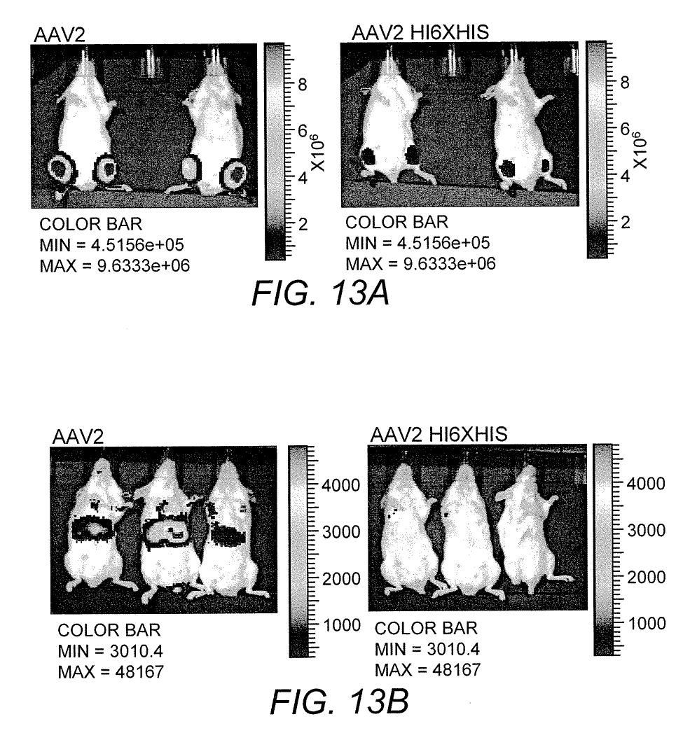

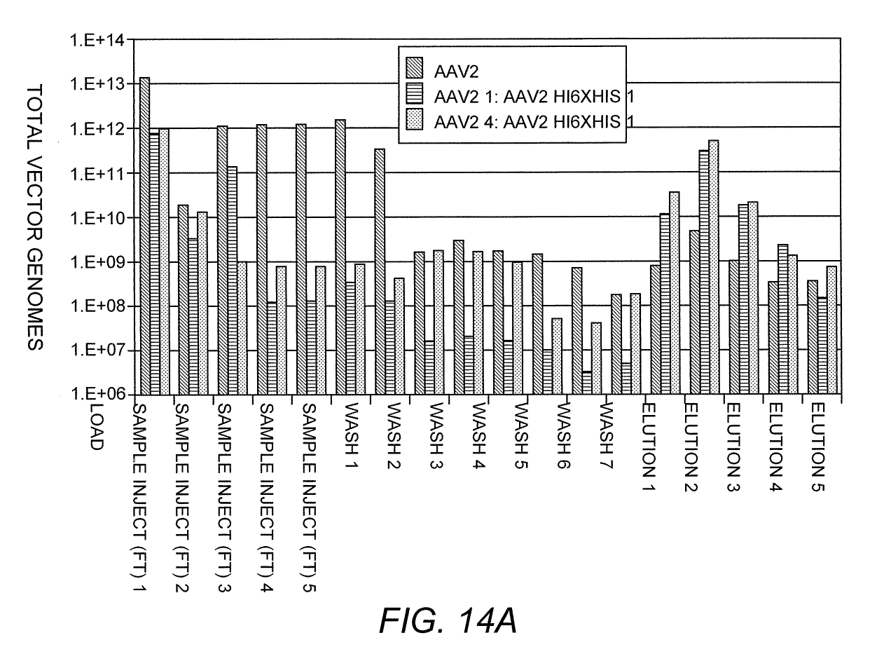

The utilization of the vector as a tool for universal purification is extremely promising but would be further validated by its ability to maintain wildtype properties in vivo. Therefore we injected our histidine tag intramuscularly (IM) and intravenously (IV) in order to monitor tropism and transduction levels as compared to wildtype AAV2 particles carrying the luciferase transgene. First, we injected AAV2 and AAV2 HI6× His IM. Contrary to the in vitro data where AAV2 HI6× His and AAV2 were similar in heparin binding and 293 cell transduction (FIG. 10), AAV2 HI6× His was ten fold lower in muscle transduction than AAV2 as determined by photon quantification (FIG. 13A) post animal imaging. Strikingly, AAV2 HI6× His was unable to transduce the liver post IV injection based on luciferase imaging (FIG. 13B).

To further substantiate this data AAV9 and AAV9 HI6× His was injected via tail vein to analyze transduction capabilities. AAV9 was able to transduce tissue systemically, as defined in the literature (Inagaki et al., Mol. Ther. 14: 45-53 (2006)). However, like AAV2 HI6× His, AAV9 HI6× His displayed reduced muscle transduction and was unable to transduce the liver based on photon quantification post live animal imaging (data not shown). Based on these data, it is believed that the hexa-histidine tag is disrupting the ability of the virus to transduce cells in vivo. In order to determine if the hexa-histidine tag was altering vector tropism, we generated chimeric vectors containing fewer hexa-histidine peptides on the capsid surface.

### Hexa-Histidine Chimeras Rescue Vector Tropism

AAV2 HI6× His contains 60 copies of the hexa-histidine tag. We generated chimeras containing fewer copies of the tag. We transfected cells with multiple ratios of pXR2 wildtype to pXR2 HI6× His, with 1:1 or 5:1, respectively. The ratios are indicators of the amount of VP incorporated containing the tag based on work previously done by Dr. Joseph Rabinowitz (Rabinowitz et al., J. Virol. 78: 4421-32 (2004)). Therefore, capsids will contain approximately 30 wildtype AAV2 VPs and 30 AAV2 HI6× His VPs or 48 wildtype AAV2 VPs and 12 AAV2 HI6× His VPs. These chimeras were generated to determine if fewer copies of the hexa-histidine tag on the capsid surface would allow for rescued transduction in the muscle and liver.

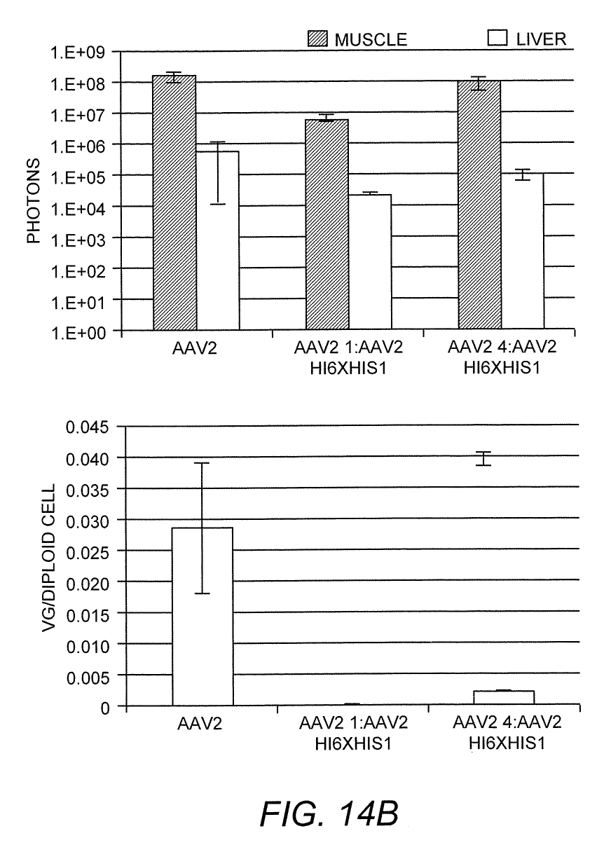

Prior to in vivo characterization of the chimeras, AAV2 1:AAV2 HI6× His 1 and AAV2 4:AAV2 HI6× His were passed through the FPLC nickel column to eliminate wildtype particles that may have been generated during virus production. Interestingly, AAV2 1:AAV2 HI6× His 1 bound and eluted from the column with a similar profile to AAV2 HI6× His and AAV2 4:AAV2 HI6× His 1 bound and eluted from the column with an elution profile similar to AAV2 and AAV2 HI6× His (FIG. 14A). Based on qPCR of eluted vector genomes, AAV2 4:AAV2 HI6× His 1 eluted from the column similar to AAV2 HI6× His in the flowthrough, and then progressively eluted in the wash fractions similar to AAV2, where increased vector genomes were detected (FIG. 14A). AAV2 4:AAV2 HI6× His 1 particles and AAV2 1:AAV2 HI6× His 1 particles eluted primarily in elution 2 (FIG. 14A) as seen with AAV2 HI6× His and AAV9 HI6× His (FIG. 11B).

Post purification, we injected our chimeric mutants containing fewer copies of the hexa-histidine tags on the capsid surface IM and IV along with AAV2 for comparison. AAV2 1:AAV2 HI6× His 1, which based on plasmid ratios during transfection of virus production should have 30 monomer copies of AAV2 and AAV2 HI6× His, behaved similarly to AAV2 HI6× His intramuscularly (FIG. 13B and data not shown), demonstrating a ten fold lower transduction as compared to wild-type AAV2 particles post photon quantification (FIG. 14B). However, AAV2 4:AAV2 HI6× His 1 shows similar transduction to AAV2 wildtype particles, with a complete rescue of virus infectivity in skeletal muscle (FIG. 14B). Therefore, the reduced number of hexa-histidine peptides on the surface of the capsid allowed for complete rescue of vector transduction in the muscle.

Interestingly, a different phenotype was observed post IV injection via the tail vein. When comparing the 1:1 ratio of AAV2:AAV2 HI6× His viral particles to the 5:1 ratio there is an increase in liver transduction (data not shown) however not to wildtype levels seen with AAV2. Muscle transduction with the 5:1 particles averaged fivefold higher than the 1:1 ratio when 1E10 vector genome containing particles were injected (FIG. 14B). Interestingly, AAV2 1:AAV2 HI6× His 1 liver transduction resembled that of AAV2 HI6× His (FIG. 13B), and there is a slight rescue of liver transduction with the 5:1 chimeric (data not shown). The transduction observed with the 5:1 chimeric is not a full rescue as determined by luminescence and vector genome quantification in the liver (data not shown).

The foregoing is illustrative of the present invention, and is not to be construed as limiting thereof. The invention is defined by the following claims, with equivalents of the claims to be included therein.

All publications, patent applications, patents, patent publications, GenBank® database sequences and other references cited herein are incorporated by reference in their entireties for the teachings relevant to the sentence and/or paragraph in which the reference is presented.

TABLE 1
Primers utilized for AAV2 HI loop mutant capsid generation

| | | |
|---|---|---|
| Primer name | Forward | Reverse |

| | | |
|---|---|---|
| AAV2 HI-/- | 5′ TCC TTC ATC ACA CAG TAC | 5′ AGG ATT CGC AGG TAC |
| | TCC ACG GGA CAG G 3′ | CGG GGT GTT CTT GAT GAG 3′ |
| AAV2 poly-glycine | 5′ GGA GGA GGA GGA GGA | TCC TCC TCC TCC TCC TCC |
| | GGA GGA GGA GGA GGA TCC | TCC TCC TCC TCC AGG ATT |
| | TTC ATC ACA CAG TAC TCC | CGC AGG TAC CGG GGT |
| | ACG GGA CAG G 3′ | GTT CTT GAT GAG 3′ |
| AAV2 poly-glycine | 5′ GCG AAT CCT GGA GGA | N/A |
| F661 | GGA TTC GGA GGA GGA GGA | |
| | GGA 3′ | |
| AAV2 poly-glycine | 5′ GGA GGA GGA GGA GGA | N/A |
| K665 | AAG GGA GGA TCC TTC ATC 3′ | |
| AAV2 poly-glycine | 5′ GGA GGA GGA GGA GGA | N/A |
| F666 | TTT GGA TCC TTC ATC ACA | |
| | CAG 3′ | |
| pXSA for HI loop | (Sbf1) 5′ AGA GAT GTG TAC | N/A |
| serotype swaps | CTG CAG GGG CCC ATC TGG 3′ | |
| | (Afe1) 5; AAG GAA AAC AGC | |
| | AAG CGC TGG AAT CCC GAA 3′ | |
| AAV5 HI loop | 5′ GAT CCT GCA GGG ACC CAT | 5′ GCT TGG AGT TTT CCT |
| | CTG GCC CAA GAT C 3′ | TCT TGA GCT CCC AC 3′ |
| AAV4 HI loop | 5′ GAT CCT GCA GGG TCC CAT 5′ GTT TGG ACC GCT CCT | |
| | TTG GGC CAA GAT T 3′ | TCT GGA TCT CCC 3′ |
| AAV2 HI5 TTSF | 5′ CCC GGA AAT ATC ACC ACC | N/A |
| | AGC TTC TCG GAC GTG 3′ | |
| pAge1 NheI | (Age1))5′ CTC ATC AAG AAC | N/A |
| | ACA CCG GTA CCT GCG 3′ | |
| | (Nhe1) 5′ GCG GCA AAG TTT | |
| | GCT AGC TTC ATC ACA CAG | |
| | TAC TCC 3′ | |
| AAV2 SIGYPLP | 5′ AAG AAC ACA CCG GTA CCT | 5′ GAT GAA GCT AGC AAA |
| | GCG MT CCT AGC ATT GGT | CTT AGG AAG AGG ATA ACC |
| | TAT CCT CTT CCT AAG TTT | AAT GCT AGG ATT CGC AGG |
| | GCT AGC TTC ATC 3′ | TAC CGG TGT GTT CTT 3′ |
| AAV2 VNTANST | 5′ AAG AAC ACA CCG GTA CCT | 5′ GAT GAA GCT AGC AAA |
| | GCG AAT CCT GTT AAT ACT | CTT AGT GCT ATT AGC AGT |
| | GCT AAT AGC ACT AAG TTT | ATT AAC AGG ATT CGC AGG |
| | GCT AGC TTC ATC 3′ | TAC CGG TGT GTT CTT 3′ |
| AAV2 QPEHSST | 5′ AAG AAC ACA CCG GTA CCT | 5′ GAT GAA GCT AGC AAA |
| | GCG AAT CCT CAA CCT GAA | CTT AGT GCT GCT ATG TTC |
| | CAT AGC AGC ACT AAG TTT | AGG TTG AGG ATT CGC |
| | GCT AGC TTC ATC 3′ | AGG TAC CGG TGT GTT CTT 3′ |
| AAV2 RGD 658 | 5′ AAG AAC ACA CCG GTA CCT | 5′ GAT GAA GCT AGC AAA |
| | GCG AAT CCT CGA GGA GAC | CTT TGC CGC ACT GAA GTC |
| | TTC AGT GCG GCA AAG TTT | TCC TCG AGG ATT CGC |
| | GCT AGC TTC ATC 3′ | AGG TAC CGG TGT CTT CTT 3′ |
| AAV2 RGD 660 | 5′ AAG AAC ACA CCG GTA CCT | 5′ GAT GAA GCT AGC AAA |
| | GCG AAT CCT TCG ACC CGA | CTT TGC CGC GTC TCC TCG |
| | GGA GAC GCG GCA AAG TTT | GGT CGA AGG ATT CGC |
| | GCT AGC TTC ATC 3′ | AGG TAC CGG TGT GTT CTT 3′ |
| AAV2 RGD 662 | 5′ AAG AAC ACA CCG GTA CCT | 5′ GAT GAA GCT AGC AAA |
| | GCG AAT CCT TCG ACC ACC | CTT GTC TCC TCG GAA GGT |
| | TTC CGA GGA GAC AAG TTT | GGT CGA AGG ATT CGC |
| | GCT AGC TTC ATC 3′ | AGG TAC CGG TGT GTT CTT 3′ |
| AAV2 RGD 663 | 5′ AAG AAC ACA CCG GTA CCT | 5′ GAT GAA GCT AGC AAA |
| | GCG AAT CCT TCG ACC ACC | GTC TCC TCG ACT GAA GGT |
| | TTC AGT CGA GGA GAC TTT | GGT CGA AGG ATT CGC |
| | GCT AGC TTC ATC 3′ | AGG TAC CGG TGT GTT CTT 3′ |
| AAV2 SGRGDS | 5′ AAG AAC ACA CCG GTA CCT | 5′ GAT GAA GCT AGC AAA |
| | GCG AAT CCT TCG GGA CGA | CTT CGC CGA GTC TCC TCG |
| | GGA GAC TCG GCG AAG TTT | TCC CGA AGG ATT CGC |
| | GCT AGC TTC ATC 3′ | AGG TAC CGG TGT GTT CTT 3′ |
| AAV2 F661G | 5′ GCG AAT CCT TCG ACC ACC | N/A |
| | GGC AGT GCG GCA AAG TTT | |
| | GCT TCC 3′ | |

TABLE 2
Phenotype comparison between AAV2 HI loop capsid mutants

| | Assembly | Packaging | | ^{a}Infectivity | Alternative VP1 | |
|---|---|---|---|---|---|---|
| | | (Western | (western | Heparin | luciferase | (Western |
| Mutant | Sequence | dot blot) | dot blot) | Binding | assay) | blot) |
| AAV2 HI-/- | (655) ANP----------SF1 | − | − | N/A | N/A | N/A |
| AAV2 poly- | (655) ANP | + | − | + | N/A | − |
| glycine | GGGGGGGGGG SF1 | | | | | |
| AAV2 G661F | (655) ANP | + | − | N/D | N/A | N/D |
| | GGGFGGGGGG SF1 | | | | | |
| AAV2 G665K | (655) ANP | + | − | N/D | N/A | N/D |
| | GGGGGGGKGG SF1 | | | | | |
| AAV2 G666F | (655) ANP | + | − | N/D | N/A | N/D |
| | GGGGGGGGFG SF1 | | | | | |
| AAV2 HI1 | (655) ANP PAEFSATKFA | + | + | + | No change | − |
| | SF1 | | | | | |
| AAV2 HI8 | (655) ANP PTTFNSQKLN | + | + | + | No change | − |
| | SF1 | | | | | |
| AAV2 HI4 | (655) ANP ATTFSSTPVN | + | − | + | No change | − |
| | SF1 | | | | | |
| AAV2 HI5 | (655) GNI T-SFSDVPVS | − | − | N/A | N/A | N/A |
| | SF1 | | | | | |
| AAV2 RGD | (655) ANP RGDFSAAKFA | + | + | N/D | No change | − |
| 658 | SF1 | | | | | |
| AAV2 RGD | (655) ANP STRGDAAKFA | + | +/− | N/D | 4.7 | + |
| 660 | SF1 | | | | | |
| AAV2 RGD | (655) ANP STTFRGDKFA | + | + | N/D | No change | − |
| 662 | SF1 | | | | | |
| AAV2 RGD | (655) ANP STTFSRGDFA | + | +/− | N/D | ^{b}No change | − |
| 663 | SF1 | | | | | |
| AAV2 | (655) ANP VNTANSTKFA | + | +/− | + | 27 | + |
| VNTANST | SF1 | | | | | |
| AAV2 | (655) ANP QPEHSSTKFA | + | + | + | 2 | + |
| QPEHSST | SF1 | | | | | |
| AAV2 | (655) ANP SIGYPLPKFA | + | +/− | N/D | 10.4 | + |
| SIGYPLP | SF1 | | | | | |
| AAV2 | (655) ANP SGRGDSAKFA | + | +/− | N/D | 4.5 | + |
| SGRGDS | SF1 | | | | | |
| AAV2 F661G | (655) ANP STTGSAAKFA | + | +/− | + | 13.55 | + |
| SF1 | | | | | | |

^{a} = Fold decrease in infectivity
^{b} = In SK-OV3 cells
+ = Similar to wt
+/− = Packaging: Approximately 5-fold lower in titer than wt
− = Packaging: One log lower in titer than wt or unable to package the viral genome
N/A = Not applicable
N/D = Not determined

TABLE 3

| VIRUS | GENBANK ACCESSION NUMBER |
|---|---|
| Complete Genomes | |
| Adeno-associated virus 1 | NC_002077, AF063497 |
| Adeno-associated virus 2 | NC_001401 |
| Adeno-associated virus 3 | NC_001729 |
| Adeno-associated virus 3B | NC_001863 |
| Adeno-associated virus 4 | NC_001829 |
| Adeno-associated virus 5 | Y18065, AF085716 |
| Adeno-associated virus 6 | NC_001862 |
| Avian AAV ATCC VR-865 | AY186198, AY629583, NC_004828 |
| Avian AAV strain DA-1 | NC_006263, AY629583 |
| Bovine AAV | NC_005889, AY388617 |
| Clade A | |
| AAV1 | NC_002077, AF063497 |
| AAV6 | NC_001862 |
| Hu. 48 | AY530611 |
| Hu 43 | AY530606 |
| Hu 44 | AY530607 |
| Hu 46 | AY530609 |
| Clade B | |
| Hu. 19 | AY530584 |
| Hu. 20 | AY530586 |
| Hu 23 | AY530589 |
| Hu22 | AY530588 |
| Hu24 | AY530590 |
| Hu21 | AY530587 |
| Hu27 | AY530592 |
| Hu28 | AY530593 |
| Hu 29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| Hu14 (AAV9) | AY530579 |
| Hu31 | AY530596 |
| Hu32 | AY530597 |
| Clonal Isolate | |
| AAV5 | Y18065, AF085716 |
| AAV 3 | NC_001729 |
| AAV 3B | NC_001863 |
| AAV4 | NC_001829 |
| Rh34 | AY243001 |
| Rh33 | AY243002 |
| Rh32 | AY243003 |

TABLE 4
Nucleic Acid and Amino Acid Sequences of AAV2, AAV9, AAV2

```
HI6xHis and AAV9 HI6xHis
AAV2 (GenBank Accession No. NC_001401)
atggctgccgatggttatcttccagattggctcgaggacactctctctgaaggaataagacagtggtgg
aagctcaaacctggcccaccaccaccaaagcccgcagagcggcataaggacgacagcaggggtcttgtg
cttcctgggtacaagtacctcggacccttcaacggactcgacaagggagagccggtcaacgaggcagac
gccgcggccctcgagcacgacaaagcctacgaccggcagctcgacagcggagacaacccgtacctcaag
tacaaccacgccgacgcggagtttcaggagcgccttaaagaagatacgtcttttgggggcaacctcgga
cgagcagtcttccaggcgaaaaagagggttcttgaacctctgggcctggttgaggaacctgttaagacg
gctccgggaaaaaagaggccggtagagcactctcctgtggagccagactcctcctcgggaaccggaaag
gcgggccagcagcctgcaagaaaaagattgaattttggtcagactggagacgcagactcagtacctgac
ccccagcctctcggacagccaccagcagccccctctggtctgggaactaatacgatggctacaggcagt
ggcgcaccaatggcagacaataacgagggcgccgacggagtgggtaattcctcgggaaattggcattgc
gattccacatggatgggcgacagagtcataccaccagcacccgaactgggccctgcccacctacaacaa
ccacctctacaaacaaatttcaagccaatcaggagcctcgaacgacaatcactactttggctacagcac
cccttgggggtattttgacttcaacagattccactgccacttttcaccacgtgactggcaaagactcat
caacaacaactggggattccgacccaagagactcaacttcaagctctttaacattcaagtcaaagaggt
cacgcagaatgacggtacgacgacgattgccaataaccttaccagcacggttcaggtgtttactgactc
ggagtaccagctcccgtacgtcctcggctcggcgcatcaaggatgcctcccgccgttcccagcagacgt
cttcatggtgccacagtatggatacctcaccctgaacaacgggagtcaggcagtaggacgctcttcatt
ttactgcctggagtactttccttctcagatgctgcgtaccggaaacaactttaccttcagctacacttt
tgaggacgttcctttccacagcagctacgctcacagccagagtctggaccgtctcatgaatcctctcat
cgaccagtacctgtattacttgagcagaacaaacactccaagtggaaccaccacgcagtcaaggcttca
gttttctcaggccggagcgagtgacattcgggaccagtctaggaactggcttcctggaccctgttaccg
ccagcagcgagtatcaaagacatctgcggataacaacaacagtgaatactcgtggactggagctaccaa
gtaccacctcaatggcagagactctctggtgaatccgggcccggccatggcaagccacaaggacgatga
agaaagttttttcctcagagcggggttctcatctttgggaagcaaggctcagagaaacaatgtggacat
tgaaaaggtcatgattacagacgaagaggaaatcaggacaaccaatcccgtggtctacggagcagtatg
gttctgtatctaccaacctccagagaggcaacagacaagcagctaccgcagatgtcaacacacaaggcg
tcttccaggcatggtctggcaggacagagatgtgtacctcaggggcccatctgggcaaagattccacac
acggacggacattttcacccctctcccctcatgggtggattcggacttaaacaccctctccacagattc
tcatcaagaacaccccggtacctgcgaatcctcgaccaccttcagtgcggcaaagtttgcttccttcat
cacacagtactccacgggacaggtcagcgtggagatcgagtgggagctgcagaaggaaaacagcaaacg
ctggaatcccgaaattcagtacacttccaactacaacaagtctgttaatgtggactttactgtggacac
taatggcgtgtattcagagcctcgccccattggcaccagatacctgactcgtaatctg
AAV2
    1 maadgylpdw ledtlsegir qwwklkpgpp ppkpaerhkd dsrglvlpgy kylgpfngld
  61 kgepvneada aalehdkayd rqldsgdnpy lkynhadaef qerlkedtsf ggnlgravfq
121 akkrvleplg lveepvktap gkkrpvehsp vepdsssgtg kagqqparkr lnfgqtgdad
181 svpdpqplgq ppaapsglgt ntmatgsgap madnnegadg vgnssgnwhc dstwmgdrvi
241 ftstrtwalp tynnhlykqi ssqsgasndn hyfgystpwg yfdfnrfhch fsprdwqrli
301 nnnwgfrpkr lnfklfniqv kevtqndgtt tiannltstv qvftdseyql pyvlgsahqg
361 clppfpadvf mvpqygyltl nngsqavgrs sfycleyfps qmlrtgnnft fsytfedvpf
421 hssyahsqsl drlmnplidq yiyylsrtnt psgtttqsrl qfsqagasdi rdqsrnwlpg
481 pcyrqqrvsk tsadnnnsey swtgatkyhl ngrdslvnpg pamashkdde ekftpqsgvl
541 ifgkqgsekt nvdiekvmit deeeirttnp vateqygsvs tnlqrgnrqa atadvntqgv
601 lpgmvwqdrd vylqgpiwak iphtdghfhp splmggfglk hpppqilikn tpvpanpstt
661 fsaakfasfi tqystgqvsv eiewelqken skrwnpeiqy tsnynksvnv dftvdtngvy
721 seprpigtry ltrnl
AAV2 HI6xHis
cggtgggcaaaggatcacgtggttgaggtggagcatgaattctacgtcaaaaagggtggagccaagaaa
agacccgcccccagtgacgcagatataagtgagcccaaacgggtgcgcgagtcagttgcgcagccatcg
acgtcagacgcggaagcttcgatcaactacgcagacaggtaccaaaacaaatgttctcgtcacgtgggc
atgaatctgatgctgtttccctgcagacaatgcgagagaatgaatcagaattcaaatatctgcttcact
cacggacagaaagacttagagtgctttcccgtgtcagaatctcaacccgtttctgtcgtcaaaaaggcg
tatcagaaactgtgctacattcatcatatcatgggaaaggtgccagacgcttgcactgcctgcgatctg
gtcaatgtggatttggatgactgcatctttgaacaataaatgatttaaatcaggtatggctgccgatgg
ttatcttccagattggctcgaggacactctctctgaaggaataagacagtggtggaagctcaaacctgg
cccaccaccaccaaagcccgcagagcggcataaggacgacagcaggggtcttgtgcttcctgggtacaa
gtacctcggacccttcaacggactcgacaagggagagccggtcaacgaggcagacgccgcggccctcga
gcacgacaaagcctacgaccggcagctcgacagcggagacaacccgtacctcaagtacaaccacgccga
cgcggagtttcaggagcgccttaaagaagatacgtcttttgggggcaacctcggacgagcagtcttcca
ggcgaaaaagagggttcttgaacctctgggcctggttgaggaacctgttaagacggctccgggaaaaaa
gaggccggtagagcactctcctgtggagccagactcctcctcgggaaccggaaaggcgggccagcagcc
tgcaagaaaaagattgaattttggtcagactggagacgcagactcagtacctgacccccagcctctcgg
acagccaccagcagccccctctggtctgggaactaatacgatggctacaggcagtggcgcaccaatggc
agacaataacgagggcgccgacggagtgggtaattcctcgggaaattggcattgcgattccacatggat
gggcgacagagtcatcaccaccagcacccgaacctgggccctgcccacctacaacaaccacctctacaa
acaaatttccagccaatcaggagcctcgaacgacaatcactactttggctacagcaccccttgggggta
ttttgacttcaacagattccactgccacttttcaccacgtgactggcaaagactcatcaacaacaactg
gggattccgacccaagagactcaacttcaagctctttaacattcaagtcaaagaggtcacgcagaatga
cggtacgacgacgattgccaataaccttaccagcacggttcaggtgtttactgactcggagtaccagct
cccgtacgtcctcggctcggcgcatcaaggatgcctcccgccgttcccagcagacgtcttcatggtgcc
acagtatggatacctcaccctgaacaacgggagtcaggcagtaggacgctcttcattttactgcctgga
gtactttccttctcagatgctgcgtaccggaaacaactttaccttcagctacacttttgaggacgttcc
tttccacagcagctacgctcacagccagagtctggaccgtctcatgaatcctctcatcgaccagtacct
gtattacttgagcagaacaaacactccaagtggaaccaccacgcagtcaaggcttcagttttctcaggc
cggagcgagtgacattcgggaccagtctaggaactggcttcctggaccctgttaccgccagcagcgagt
atcaaagacatctgcggataacaacaacagtgaatactcgtggactggagctaccaagtaccacctcaa
tggcagagactctctggtgaatccgggcccggccatggcaagccacaaggacgatgaagaaaagttttt
tcctcagagcggggttctcatctttgggaagcaaggctcagagaaaacaaatgtggacattgaaaaggt
catgattacagacgaagaggaaatcaggacaaccaatcccgtggctacggagcagtatggttctgtatc
taccaacctccagagaggcaacagacaagcagctaccgcagatgtcaacacacaaggcgttcttccagg
catggtctggcaggacagagatgtgtaccttcaggggcccatctgggcaaagattccacacacggacgg
acattttcacccctctcccctcatgggtggattcggacttaaacaccctcctccacagattctcatcaa
gaacaccccggtacctgcgaatccttcgaccaccttc caccatcaccatcaccat
tccttcatcacacagtactccacgggacaggtcagcgtggagatcgagtgggagctgcagaaggaaaac
agcaaacgctggaatcccgaaattcagtacacttccaactacaacaagtctgttaatgtggactttact
gtggacactaatggcgtgtattcagagcctcgccccattggcaccagatacctgactcgtaatctgtaa
ttgcttgttaatcaataaaccgtttaattcgtttcagttgaactttggtgtcgcggccgctcgataagc
ttttgttccctttagtgagggttaattccgagcttggcgtaatcatggtcatagctgtttcctgtgtga
aattgttatccgctcacaattccacacaacatacgagccggaagcataaagtgtaaagcctggggtgcc
taatgagtgagctaactcacattaattgcgttgcgctcactgcccgctttccagtcgggaaacctgtcg
tgccagctgcattaatgaatcggccaacgcgcggggagaggcggtttgcgtattgggcgctcttccgct
tcctcgctcactgactcgctgcgctcggtcgttcggctgcggcgagcggtatcagctcactcaaaggcg
gtaatacggttatccacagaatcaggggataacgcaggaaagaacatgtgagcaaaaggccagcaaaag
gccaggaaccgtaaaaaggccgcgttgctggcgtttttccataggctccgcccccctgacgagcatcac
aaaaatcgacgctcaagtcagaggtggcgaaacccgacaggactataaagataccaggcgtttccccct
ggaagctccctcgtgcgctctcctgttcc
gaccctgccgcttaccggatacctgtccgccttctcccttcgggaagcgtggcgctttctcatagctca
cgctgtaggtatctcagttcggtgtaggtcgttcgctccaagctgggctgtgtgcacgaaccccccgtt
cagcccgaccgctgcgccttatccggtaactatcgtcttgagtccaacccggtaagacacgacttatcg
ccactggcagcagccactggtaacaggattagcagagcgaggtatgtaggcggtgctacagagttcttg
aagtggtggcctaactacggctacactagaaggacagtatttggtatctgcgctctgctgaagccagtt
accttcggaaaaagagttggtagctcttgatccggcaaacaaaccaccgctggtagcggtggttttttt
gtttgcaagcagcagattacgcgcagaaaaaaaggatctcaagaagatcctttgatcttttctacgggg
tctgacgctcagtggaacgaaaactcacgttaagggattttggtcatgagattatcaaaaaggatcttc
acctagatccttttaaattaaaaatgaagttttaaatcaatctaaagtatatatgagtaaacttggtct
gacagttaccaatgcttaatcagtgaggcacctatctcagcgatctgtctatttcgttcatccatagtt
gcctgactccccgtcgtgtagataactacgatacgggagggcttaccatctggccccagtgctgcaatg
ataccgcgagacccacgctcaccggctccagatttatcagcaataaaccagccagccggaagggccgag
cgcagaagtggtcctgcaactttatccgcctccatccagtctattaattgttgccgggaagctagagta
agtagttcgccagttaatagtttgcgcaacgttgttgccattgctacaggcatcgtggtgtcacgctcg
tcgtttggtatggcttcattcagctccggttcccaacgatcaaggcgagttacatgatcccccatgttg
tgcaaaaaagcggttagctccttcggtcctccgatcgttgtcagaagtaagttggccgcagtgttatca
ctcatggttatggcagcactgcataattctcttactgtcatgccatccgtaagatgcttttctgtgact
ggtgagtactcaaccaagtcattctgagaatagtgtatgcggcgaccgagttgctcttgcccggcgtca
atacgggataataccgcgccacatagcagaactttaaaagtgctcatcattggaaaacgttcttcgggg
cgaaaactctcaaggatcttaccgctgttgagatccagttcgatgtaacccactcgtgcacccaactga
tcttcagcatctttactttcaccagcgtttctgggtgagcaaaaacaggaaggcaaaatgccgcaaaaa
agggaataagggcgacacggaaatgttgaatactcatactcttcctttttcaatattattgaagcattt
atcagggttattgtctcatgagcggatacatatttgaatgtatttagaaaaataaacaaataggggttc
cgcgcacatttccccgaaaagtgccacctgacgtctaagaaaccattattatcatgacattaacctata
aaaataggcgtatcacgaggccctttcgtctcgcgcgtttcggtgatgacggtgaaaacctctgacaca
tgcagctcccggagacggtcacagcttgtctgtaagcggatgccgggagcagacaagcccgtcagggcg
cgtcagcgggtgttggcgggtgtcggggctggcttaactatgcggcatcagagcagattgtactgagag
tgcaccatatgcggtgtgaaataccgcacagatgcgtaaggagaaaataccgcatcaggaaattgtaaa
cgttaatattttgttaaaattcgcgttaaatttttgttaaatcagctcattttttaaccaataggccga
aatcggcaaaatcccttataaatcaaaagaatagaccgagatagggttgagtgttgttccagtttggaa
caagagtccactattaaagaacgtggactccaacgtcaaagggcgaaaaaccgtctatcagggcgatgg
cccactacgtgaaccatcaccctaatcaagttttttggggtcgaggtgccgtaaagcactaaatcggaa
ccctaaagggagcccccgatttagagcttgacggggaaagccggcgaacgtggcgaggaaggaagggaa
gaaagcgaaaggagcgggcgctagggcgctggcaagtgtagcggtcacgctgcgcgtaaccaccacacc
cgccgcgcttaatgcgccgctacagggcgcgtcgcgccattcgccattcaggctgcgcaactgttggga
agggcgatcggtgcgggcctcttcgctattacgccagctggcgaaagggggatgtgctgcaaggcgatt
aagttgggtaacgccagggttttcccagtcacgacgttgtaaaacgacggccagtgaattgtaatacga
ctcactatagggcgaattcgagctcggtacccctagagtcctgtattagaggtcacgtgagtgttttgc
gacattttgcgacaccatgtggtcacgctgggtatttaagcccgagtgagcacgcagggtctccatttt
gaagcgggaggtttgaacgcgcagccgccatgccggggttttacgagattgtgattaaggtccccagcg
accttgacgggcatctgcccggcatttctgacagctttgtgaactgggtggccgagaaggaatgggagt
tgccgccagattctgacatggatctgaatctgattgagcaggcacccctgaccgtggccgagaagctgc
agcgcgactttctgacggaatggcgccgtgtgagtaaggccccggaggcccttttctttgtgcaatttg
agaagggagagagctacttccacatgcacgtgctcgtggaaaccaccggggtgaaatccatggttttgg
gacgtttcctgagtcagattcgcgaaaaactgattcagagaatttaccgcgggatcgagccgactttgc
caaactggttcgcggtcacaaagaccagaaatggcgccggaggcgggaacaaggtggtggatgagtgct
acatccccaattacttgctccccaaaacccagcctgagctccagtgggcgtggactaatatggaacagt
atttaagcgcctgtttgaatctcacggagcgtaaacggttggtggcgcagcatctgacgcacgtgtcgc
agacgcaggagcagaacaaagagaatcagaatcccaattctgatgcgccggtgatcagatcaaaaactt
cagccaggtacatggagctggtcgggtggctcgtggacaaggggattacctcggagaagcagtggatcc
aggaggaccaggcctcatacatctccttcaatgcggcctccaactcgcggtcccaaatcaaggctgcct
tggacaatgcgggaaagattatgagcctgactaaaaccgcccccgactacctggtgggccagcagcccg
tggaggacatttccagcaatcggatttataaaattttggaactaaacgggtacgatccccaatatgcgg
cttccgtctttctgggatgggccacgaaaaagttcggcaagaggaacaccatctggctgtttgggcctg
caactaccgggaagaccaacatcgcggaggccatagcccacactgtgcccttctacgggtgcgtaaact
ggaccaatgagaactttcccttcaacgactgtgtcgacaagatggtgatctggtgggaggaggggaaga
tgaccgccaaggtcgtggagtcggccaaagccattctcggaggaagcaaggtgcgcgtggaccagaaat
gcaatcctcggcccagatagacccgactcccgtgatcgtcacctccaacaccaacatgtgcgccgtgat
tgacgggaactcaacgaccttcgaacaccagcagccgttgcaagaccggatgttcaaatttgaactcac
ccgccgtctggatcatgactttgggaaggtcaccaagcaggaagtcaaagactttttc
AAV2 HI6xHis (SEQ ID NO:1)
    1 maadgylpdw ledtlsegir qwwklkpgpp ppkpaerhkd dsrglvlpgy kylgpfngid
  61 kgepvneada aalehdkayd rqldsgdnpy lkynhadaef qerlkedtsf ggnlgravtq
121 akkrvleplg lveepvktap gkkrpvehsp vepdsssgtg kagqqparkr lnfgqtgdad
181 svpdpqplgq ppaapsglgt ntmatgsgap madnnegadg vgnssgnwhc dstwmgdrvi
241 ttstrtwalp tynnhlykqi ssqsgasndn hyfgystpwg yfdfnrfhch fsprdwqrii
301 nnnwgfrpkr lnfklfniqv kevtqndgtt tiannltstv qvftdseyql pyvlgsahqg
361 clppfpadvf mvpqygyltl nngsqavgrs sfycleyfps qmlrtgnnft fsytfedvpf
421 hssyahsqsl drlmnplidq ylyylsrtnt psgtttqsrl qfsqagasdi rdqsrnwlpg
481 pcyrqqrvsk tsadnnnsey swtgatkyhl ngrdslvnpg pamashkdde ekffpqsgvl
541 ifgkqgsekt nvdiekvmit deeeirttnp vateqygsvs tnlqrgnrqa atadvntqgv
601 lpgmvwqdrd vylqgpiwak iphtdghfhp splmggfglk hpppqilikn tpvpanpstt
661 fHHHHHHsfi tqystgqvsv eiewelqken skrwnpeiqy tsnynksvnv dftvdtngvy
721 seprpigtry ltrnl
AAV9 (GenBank Accession No. AY530579)
atggctgccgatggttatcttccagattggctcgaggacaaccttagtgaaggaattcgcgagtggtgg
gctttgaaacctggagcccctcaacccaaggcaaatcaacaacatcaagacaacgctcgaggtcttgtg
cttccgggttacaaataccttggacccggcaacggactcgacaagggggagccgtcaacgcagcagacg
cggcggccctcgagcacgacaaggcctacgaccagcagctcaaggccggagacaacccgtacctcaagt
acaaccacgccgacgccgagttccaggagcggctcaaagaagatacgtcttttgggggcaacctcgggc
gagcagtcttccaggccaaaaagaggcttcttgaacctcttggtctggttgaggaagcggctaagacgg
ctcctggaaagaagaggcctgtagagcagtctcctcaggaaccggactcctccgcgggtattggcaaat
cgggtgcacagcccgctaaaaagagactcaatttcggtcagactggcgacacagagtcagtcccagacc
ctcaaccaatcggagaacctcccgcagccccctcaggtgtgggatctcttacaatggcttcaggtggtg
gcgcaccagtggcagacaataacgaaggtgccgatggagtgggtagttcctcgggaaattggcattgcg
attcccaatggctgggggacagagtcatcaccaccagcacccgaacctgggccctgcccacctacaaca
atcacctctacaagcaaatctccaacagcacatctggaggatcttcaaatgacaacgcctacttcggct
acagcaccccctgggggtattttgacttcaacagattccactgccacttctcaccacgtgactggcagc
gactcatcaacaacaactggggattccggcctaagcgactcaacttcaagctcttcaacattcaggtca
aagaggttacggacaacaatggagtcaagaccatcgccaataaccttaccagcacggtccaggtcttca
cggactcagactatcagctcccgtacgtgctcgggtcggctcacgagggctgcctcccgccgttcccag
cggacgttttcatgattcctcagtacgggtatctgacgcttaatgatggaagccaggccgtgggtcgtt
cgtccttttactgcctggaatatttcccgtcgcaaatgctaagaacgggtaacaacttccagttcagct
acgagtttgagaacgtacctttccatagcagctacgctcacagccaaagcctggaccgactaatgaatc
cactcatcgaccaatacttgtactatctctcaaagactattaacggttctggacagaatcaacaaacgc
taaaattcagtgtggccggacccagcaacatggctgtccagggaagaaactacatacctggacccagct
accgacaacaacgtgtctcaaccactgtgactcaaaacaacaacagcgaatttgcttggcctggagctt
cttcttgggctctcaatggacgtaatagcttgatgaatcctggacctgctatggccagccacaaagaag
gagaggaccgtttctttcctttgtctggatctttaatttttggcaaacaaggaactggaagagacaacg
tggatgcggacaaagtcatgataaccaacgaagaagaaattaaaactactaacccggtagcaacggagt
cctatggacaagtggccacaaaccaccagagtgcccaagcacaggcgcagaccggctgggttcaaaacc
aaggaatacttccgggtatggtttggcaggacagagatgtgtacctgcaaggacccatttgggccaaaa
ttcctcacacggacggcaactttcacccttctccgctgatgggagggtttggaatgaagcacccgcctc
ctcagatcctcatcaaaaacacacctgtacctgcggatcctccaacggccttcaacaaggacaagctga
actctttcatcacccagtattctactggccaagtcagcgtggagatcgagtgggagctgcagaaggaaa
acagcaagcgctggaacccggagatccagtacacttccaactattacaagtctaataatgttgaatttg
ctgttaatactgaaggtgtatatagtgaaccccgccccattggcaccagatacctgactcgtaatctgt
aa
AAV9
    1 maadgylpdw lednisegir ewwalkpgap qpkanqqhqd narglvlpgy kylgpgngld
  61 kgepvnaada aalehdkayd qqlkagdnpy lkynhadaef qerlkedtsf ggnlgravfq
121 akkrlleplg lveeaaktap gkkrpveqsp qepdssagig ksgaqpakkr lnfgqtgdte
181 svpdpqpige ppaapsgvgs Itmasgggap vadnnegadg vgsssgnwhc dsqwlgdrvi
241 ttstrtwaip tynnhlykqi snstsggssn dnayfgystp wgyfdfnrfh chfsprdwqr
301 linnnwgfrp krlnfklfni qvkevtdnng vktiannlts tvqvftdsdy qlpyvlgsah
361 egclppfpad vtmipqygyl tlndgsqavg rssfycleyf psqmirtgnn fqfsyefenv
421 pfhssyahsq sldrlmnpli dqylyylskt ingsgqnqqt lkfsvagpsn mavqgrnyip
481 gpsyrqqrvs ttvtqnnnse fawpgasswa lngrnslmnp gpamashkeg edrffplsgs
541 lifgkqgtgr dnvdadkvmi tneeeikttn pvatesygqv atnhqsaqaq aqtgwvqnqg
601 ilpgmvwqdr dvylqgpiwa kiphtdgnfh pspimggfgm khpppqilik ntpvpadppt
661 afnkdklnsf itqystgqvs veiewelqke nskrwnpeiq ytsnyyksnn vefavntegv
721 yseprpigtr yltrnl
AAV9 HI6xHis
atggctgccgatggttatcttccagattggctcgaggacaaccttagtgaaggaattcgcgagtggtgg
gctttgaaacctggagcccctcaacccaaggcaaatcaacaacatcaagacaacgctcgaggtcttgtg
cttccgggttacaaataccttggacccggcaacggactcgacaagggggagccggtcaacgcagcagac
gcggcggccctcgagcacgacaaggcctacgaccagcagctcaaggccggagacaacccgtacctcaag
tacaaccacgccgacgccgagttccaggagcggctcaaagaagatacgtcttttgggggcaacctcggg
cgagcagtcttccaggccaaaagaggcttcttgaacctcttggtctggttgaggaagcggctaagacgg
ctcctggaaagaagaggcctgtagagcagtctcctcaggaaccggactcctccgcgggtatgtggcaaa
tcgggtgcacagcccgctaaaaagagactcaatttcggtcagactggcgacacagagtcagtcccagac
cctcaaccaatcggagaacctcccgcagccccctcaggtgtgggatctcttacaatggcttcaggtggt
ggcgcaccagtggcagacaataacgaaggtgccgatggagtgggtagttcctcgggaaattggcattgc
gattcccaatggctgggggacagagtcatcaccaccagcacccgaacctgggccctgcccacctacaac
aatcacctctacaagcaaatctccaacagacactctggaggatcttcaaatgacaacgcctacttcggc
tacagcaccccctgggggtattttgacttcaacagattccactgccacttctcaccacgtgactggcag
cgactcatcaacaacaactggggattccggcctaagcgactaacttcaagctcttcaacattcaggtca
aagaggttacggacaacaatggagtcaagaccatcgccaataaccttaccagcacggtccaggtcttca
cggactcagactatcagctcccgtacgtgctcgggtcggctcacgagggctgcctcccgccgttcccag
cggacgttttcatgattcctcagtacgggtatctgacgcttaatgatggaagccaggccgtgggtcgtt
cgtccttttactgcctggaatatttcccgtcgcaaatgctaagaacgggtaacaacttccagttcagct
acgagtttgagaacgtacctttccatagcagctacgctcacagccaaagcctggaccgactaatgaatc
cactcatcgaccaatacttgtactatctctcaaagactattaacggttctggacagaatcaacaaacgc
taaaattcagtgtggccggacccagcaacatggctgtccagggaacaaactacatacctggacccagct
accgacaacaacgtgtctcaaccactgtgactcaaaacaacaacagcgaatttgcttggcctggagctt
cttcttgggctctcaatggacgtaatagcttgatgaatcctggacctgctatggccagccacaaagaag
gagaggaccgtttctttcctttgtctggatctttaatttttggcaaacaaggaactggaagagacaacg
tggatgcggacaaagtcatgataaccaacgaagaagaaattaaaactactaacccggtagcaacggagt
cctatggacaagtggccacaaaccaccagagtgcccaagcacaggcgcagaccggctgggttcaaaacc
aaggaatacttccgggatggtttggcaggacagagatgtgtacctgcaaggacccatttgggccaaaat
tcctcacacggacggcaactttcacccttctccgctgatgggagggtttggaatgaagcacccgcctcc
tcagatcctcatcaaaaacacacctgtacctgcggatcctccaacggccttc catcaccaceatcacc
attctttcatcacccagtattctactggccaagtcagcgtggagatcgagtgggagctgcagaaggaaa
acagcaagcgctggaacccggagatccagtacacttccaactattacaagtctaataatgttgaatttg
ctgttaatactgaaggtgtatatagtgaaccccgccccattggcaccagatacctgactcgtaatctgt
aa
AAV9 HI6xHis (SEQ ID NO:2)
    1 maadgylpdw lednisegir ewwalkpgap qpkanqqhqd narglvlpgy kylgpgngld
  61 kgepvnaada aalehdkayd qqlkagdnpy lkynhadaef qerlkedtsf ggnlgravfq
121 akkrlleplg iveeaaktap gkkrpveqsp qepdssagig ksgaqpakkr Infgqtgdte
181 svpdpqpige ppaapsgvgs Itmasgggap vadnnegadg vgsssgnwhc dsqwlgdrvi
241 ttstrtwalp tynnhlykqi snstsggssn dnayfgystp wgyfdfnrfh chfsprdwqr
301 Iinnnwgfrp krlnfkifni qvkevtdnng vktiannlts tvqvftdsdy qlpyvlgsah
361 egclppfpad vfmipqygyl tlndgsqavg rssfycleyf psqmlrtgnn fqfsyefenv
421 pfhssyahsq sldrlmnpli dqylyylskt ingsgqnqqt lkfsvagpsn mavqgrnyip
481 gpsyrqqrvs ttvtqnnnse fawpgasswa Ingrnslmnp gpamashkeg edrffplsgs
541 lifgkqgtgr dnvdadkvmi tneeeikttn pvatesygqv atnhqsaqaq aqtgwvqnqg
601 ilpgmvwqdr dvylqgpiwa kiphtdgnfh psplmggfgm khpppqilik ntpvpadppt
661 afHHHHHHsf itqystgqvs veieweiqke nskrwnpeiq ytsnyyksnn vefavntegv
721 yseprpigtr yltrnl
```

TABLE 5
Sequence Alignment of HI Loop from Different AAV Serotypes

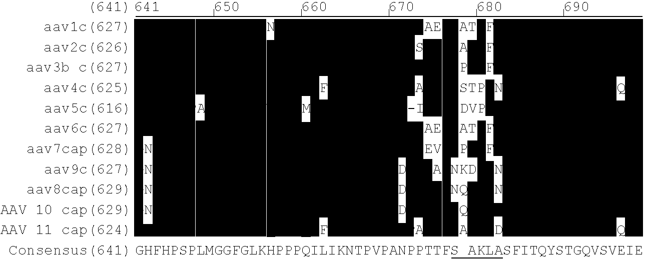

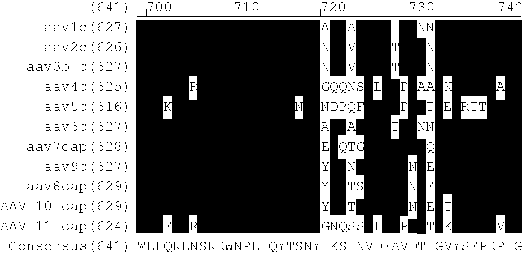

TABLE 6

Corresponding HI Loop Regions
in Different AAV Serotypes

| | | Amino Acid Position |
|---|---|---|
| | Sequence | (VP1 Numbering) |
| AAV1 | SATKFA | 663-668 |
| AAV2 | SAAKFA | 662-667 |
| AAV3b | SPAKFA | 663-668 |
| AAV4 | SSTPVN | 661-666 |
| AAV5 | SDVPVS | 651-656 |
| AAV6 | SSATKFA | 663-668 |
| AAV7 | SATKFA | 664-669 |
| AAV8 | NQSKLN | 665-670 |
| AAV9 | NKDKLN | 663-668 |
| AAV10 | SQAKLA | 665-670 |
| AAV11 | TAARVD | 660-665 |

(sequence information from Table 5)

What is claimed is:

1. An adeno-associated virus (AAV) capsid protein comprising one or more amino acid substitutions in the HI loop of the AAV capsid protein, wherein the amino acid substitution is in the region of amino acid positions 658 through 667 of the native AAV2 capsid protein or the corresponding positions of the capsid subunit of another AAV.

2. The AAV capsid protein of claim 1, wherein the AAV capsid protein is an AAV2 capsid protein.

3. The AAV capsid protein of claim 1, wherein the AAV capsid protein is an AAV9 capsid protein.

4. The AAV capsid protein of claim 1, wherein the amino acid substitution is in the region of amino acid positions 662 through 667 of the native AAV2 capsid protein or the corresponding positions of the capsid subunit of another AAV.

5. The AAV capsid protein of claim 1, wherein 3 to 6 amino acids are substituted.

6. The AAV capsid protein of claim 1, wherein the amino acid substitution comprises a sequence that facilitates detection.

7. The AAV capsid protein of claim 1, wherein the amino acid substitution comprises an affinity tag.

8. The AAV capsid protein of claim 1, wherein the amino acid substitution comprises a targeting sequence.

9. The AAV capsid protein of claim 1, wherein the amino acid substitution comprises a non-naturally occurring amino acid.

10. The AAV capsid protein of claim 1, wherein the amino acid substitution comprises a substitution of at least 4 histidine residues.

11. The AAV capsid protein of claim 10, wherein the amino acid substitution comprises a substitution of at least 4 contiguous histidine residues in the region of amino acid positions 662 through 667 of the native AAV2 capsid protein or the corresponding positions of the capsid subunit of another AAV.

12. The AAV capsid protein of claim 11, wherein the amino acid substitution comprises a substitution of a histidine residue at amino acids 662 through 667 in a native AAV2 capsid protein or the corresponding positions in a capsid protein from another AAV.

13. The AAV capsid protein of claim 12, wherein the AAV capsid protein has the amino acid sequence of SEQ ID NO:1 (AAV2 HI6× His).

14. The AAV capsid protein of claim 12, wherein the AAV capsid protein has the amino acid sequence of SEQ ID NO:2 (AAV9 HI6× His).

15. An AAV vector comprising the AAV capsid protein of claim 1.

16. The AAV vector of claim 15, wherein the AAV vector comprises 60 copies of the capsid protein.

17. The AAV vector of claim 15, wherein the AAV vector comprises 30 copies of the capsid protein.

18. The AAV vector of claim 15, wherein the AAV vector comprises 12 copies of the capsid protein.

19. An AAV vector comprising the AAV capsid protein of claim 10.

20. The AAV vector of claim 19, wherein the AAV vector comprises 60 copies of the capsid protein.

21. The AAV vector of claim 19, wherein the AAV vector comprises 30 copies of the capsid protein.

22. The AAV vector of claim 19, wherein the AAV vector comprises 12 copies of the capsid protein.

23. The AAV vector of claim 19, wherein the AAV vector has enhanced binding affinity to nickel as compared with an AAV vector that lacks a capsid protein comprising the histidine substitution.

24. The AAV capsid protein of claim 10, wherein the capsid protein is conjugated to a gold nanoparticle.

25. An AAV vector comprising the capsid protein of claim 24.

26. A pharmaceutical formulation comprising the AAV vector of claim 15 in a pharmaceutically acceptable carrier.

27. A method of administering a nucleic acid to a cell comprising contacting the cell with the AAV vector of claim 15.

28. A method of delivering a nucleic acid to a subject comprising administering to the subject the AAV vector of claim 15.

29. The method of claim 28, wherein the subject is a human subject.

30. A method of modulating the tissue tropism of an AAV vector in a subject comprising administering to the subject the AAV vector of claim 15.

31. The method of claim 30, wherein the modulation of tissue tropism is detargeting of the AAV vector from liver tissue.

32. A method of purifying an adeno-associated virus (AAV) vector from a sample, the method comprising:
(a) providing a solid support comprising a matrix, wherein the matrix comprises nickel;
(b) contacting the solid support with a sample comprising the AAV vector of claim 19; and
(c) eluting the bound AAV vector from the matrix.

33. The method of claim 32, wherein the solid support is provided in a chromatography column.
